(19) United States
(12) Patent Application Publication
(10) Pub. No.: US 2012/0207742 A1
(43) Pub. Date: Aug. 16, 2012

(54) Treatments Using PSMA Ligand Endopeptidases

(75) Inventors: Birgitte P.S. Jacky, Orange, CA (US); Patton E. Garay, Long Beach, CA (US); Yanira Molina, Tustin, CA (US); Joseph Francis, Laguna Niguel, CA (US); Lance E. Steward, Irvine, CA (US); Sanjiv Ghanshani, Irvine, CA (US); Terrence J. Hunt, Corona, CA (US); George Sachs, Encino, CA (US); Kei Roger Aoki, Coto de Caza, CA (US); Ester Fernandez-Salas, Fullerton, CA (US)

(73) Assignee: ALLERGAN, INC., IRVINE, CA (US)

(21) Appl. No.: 13/371,676

(22) Filed: Feb. 13, 2012

Related U.S. Application Data
(60) Provisional application No. 61/442,757, filed on Feb. 14, 2011.

(51) Int. Cl. A61K 38/46; A61P 35/04; A61P 35/00

(52) U.S. Cl. 424/94.67

(57) ABSTRACT

The present specification discloses TVEMPs, compositions comprising such TVEMPs and methods of treating a prostate cancer, a benign prostatic hyperplasia, and/or neovascularization or pathological angiogenesis associated with a cancer in a mammal using such TVEMP compositions.

This application claims priority pursuant to 35 U.S.C. §119(e) to U.S. Provisional Patent Application Ser. No. 61/442,757, filed Feb. 14, 2011, incorporated entirely by reference.

Cancer is a group of more than 100 diseases in which a group of cells display uncontrolled growth (cell division beyond the normal limits). In most cases, cancer cells form a clump of cells called a tumor, although in some cancers, like leukemia, the cells do not form tumors. Tumors may be malignant or benign. Besides, malignant tumors (or cancers) comprise cells with abnormal genetic material and usually undergo rapid uncontrolled cell growth, invade and destroy adjacent tissue, and sometimes spread to other locations in the body via lymph or blood (i.e., metastasis). Cancer is associated with a high incidence of mortality because if the invasion and metastasis of the cancer cells throughout the body are not stopped, cancer cells will invade vital organs and lead to the dysfunction of the organs and eventual death. The malignant properties of cancers differentiate them from benign tumors, which are usually slow-growing and self-limited, do not invade or metastasize, and as such, are generally not life-threatening. Cancers at the local, regional or distant stage are considered invasive. A very early cancer found in only a few layers of cells, called in situ cancer, is considered non-invasive.

Cancer is a diverse class of diseases which differ widely in their causes and biology. Cancers are caused by a variety of factors working alone or in combination. Some cancers are caused by external factors such as tobacco, diet, certain chemicals, radiation, and viruses. Other cancers are caused by internal factors such as hormones, immune conditions, and inherited genetic mutations. Usually ten or more years pass between exposure to a factor that causes cancer and detectable disease.

Cancers are generally classified by the type of cell that resembles the tumor and, therefore, the tissue presumed to be the origin of the tumor. Carcinomas are malignant tumors derived from epithelial cells. This group represents the most common cancers, including the common forms of breast, prostate, lung and colon cancer. Sarcomas are malignant tumors derived from connective tissue, or mesenchymal cells. Blastomas are usually malignant tumors which resemble an immature or embryonic tissue. Many of these tumors are most common in children. Lymphomas and leukemias are malignancies derived from hematopoietic (blood-forming) cells. Lastly, germ cell tumors are tumors derived from totipotent cells. In adults most often found in the testicle and ovary; in fetuses, babies, and young children most often found on the body midline, particularly at the tip of the tailbone.

Cancer is the second leading cause of death in the U.S. According to American Cancer Society projections, there were 1,479,350 new cases of cancer in 2009, including 192,280 prostate cancers; 194,280 female breast cancers; 219,440 lung cancers; and 146,970 cancers of the colon/rectum. In 2009 there were 562,340 cancer deaths overall, including 159,390 deaths from lung cancer; 49,920 from cancers of the colon/rectum; 40,610 from female breast cancer; 35,240 deaths from cancer of the pancreas (replacing prostate cancer as the fourth leading cause); and 27,360 from prostate cancer over the past 50 years, the death rate from cancer has increased steadily, due mainly to a large rise in lung cancer death rates resulting from smoking. Cancer occurs in people of all ages, but its occurrence increases greatly in people over 45 years of age. However, cancer is the leading cause of death in the United States for people between the ages of 35 and 65 and it is also the leading cause of non-accidental death among U.S. children under age 15. Men have a higher mortality rate due to cancer than women, and blacks have the highest cancer mortality rate of any major racial group. In the U.S., men have about a 1 in 2 lifetime risk of developing cancer and women have about a 1 in 3 lifetime risk. With the anticipated continued decrease in deaths from heart disease and strokes, cancer will become the overall leading cause of death for the entire American population by the year 2010.

Diagnosis of cancer usually requires a histological examination of a tissue biopsy specimen by a pathologist, although the initial indication of malignancy can be symptoms or radiographic imaging abnormalities. Once diagnosed, cancer is commonly treated by surgery, chemotherapy, radiotherapy, or targeted therapies like immunotherapy, hormonal therapy, or angiogenesis inhibitor therapy. The choice of therapy depends upon the location and grade of the tumor and the stage of the disease, as well as the general state of the patient (performance status). Furthermore, depending on the type and stage of the cancer, two or more of these types of cancer treatments may be combined at the same time or used after one another. Although complete removal of the cancer without damage to the rest of the body is the goal of treatment, current approaches to treating cancer have met with limited success. With respect to surgery, this is due, in part, to the propensity of individual or small numbers of cancer cells to invade adjacent tissue or metastasis to distant sites, thereby limiting the effectiveness of local surgical treatments. The effectiveness of chemotherapy and radiotherapy is often limited by toxicity to or damage of normal tissues in the body. Although targeted therapies are promising, as implied by their name, these treatments are usually specific for one particular type of cancer. Therefore, compounds and methods that can target all cancer cells, regardless of their location would be highly desirable for the treatment of cancer. In addition, compounds and methods that can target a particular type of cancer for which no current targeted therapy exists would also be highly desirable.

Prostate cancer is the most common male malignancy in western society, amounting to about 192,280 new cases/year in the US. More males die from prostate cancer (>30,000/year) than any other malignancy except lung cancer, and the cumulative cost of treating patients has been estimated at $8-10 billion/year in the US. Advanced stages of prostate cancer can also significantly impact quality of life due to bone disintegration, pain, obstruction of urination, and erectile dysfunction among other disorders.

Benign prostatic hyperplasia (BPH), also known as prostate enlargement, is a condition commonly seen in men over 40 and particularly in older men. While BPH is not synonymous with clinical disease, prostatic enlargement with underlying hyperplasia is a major determinant of lower urinary tract symptoms (LUTS). According to the American National Institutes of Health (NIH), BPH affects more than 50% of men over 60 and as many as 90% of men over 70. The socio-economic costs of BPH are tremendous. Three out of four men over the age of 70 will have some degree of LUTS, and more than half of these will have moderate to severe symptoms. The annual medical expenditure of BPH in the United States, including medical services and pharmaceuticals, is about $4 billion, approximately twice that of prostate cancer. Although BPH is almost never fatal, the morbidity of LUTS, and the potential complications due to BPH place a great burden on patients as well as the health care system. It is currently believed that BPH is intrinsically a mesenchymal disease that results from a reawakening of embryonic inductive and reciprocal interactions between the prostatic stroma and epithelium. For example, a number of growth factors and cytokines are over expressed in BPH stroma, including fibroblast growth factor 2 and 7 (FGF-2, FGF-7), Insulin-like growth factor 1 and 2 (IGF-1, IGF-2), and interleukin-1α (IL-1α). The increased expression of growth factors is believed to be the lead trigger for the overgrowth of epithelial and stromal cells in BPH.

The observed up-regulation of cytokines and growth factors in BPH, suggest that a silencing of these factors could be beneficial in the treatment of BPH. The histopathology of BPH strongly implicates local paracrine and autocrine growth factors and inflammatory cytokines in its pathogenesis. A complex milieu of growth-regulatory proteins includes members of the fibroblast, insulin-like, and transforming growth factor families. It appears that these proteins and downstream effector molecules, in addition to a variety of interleukins, are overexpressed in BPH and, working together, create a landscape of increased stromal and epithelial growth and mesenchymal transdifferentiation that leads to disease progression. The maintenance of autocrine and paracrine loops relies on the presence of receptors in the cell membrane to receive the extracellular signals and transduce the message to the cells. Inhibiting delivery of key receptors involved in cell proliferation and survival to the plasma membrane could be beneficial in the treatment of BPH. Moreover, inflammation, commonly present in BPH, may contribute to tissue injury, and cytokines produced by inflammatory cells may serve to drive local growth factor production and angiogenesis in the tissues as a “wound healing” response. The inhibition of the secretion of chemoattractants for immune cells will result on decrease infiltration of immune cells and lower levels of inflammation in BPH that could be beneficial in the treatment of BPH.

Tumors require a vascular supply to grow. Without blood vessels, tumors cannot grow beyond a critical size or metastasize to another organ and blocking angiogenesis can suppress tumor growth. It is known that tumor vessels develop by sprouting or intussusceptions from pre-existing vessels. Circulating endothelial precursors, shed from the vessel wall or mobilized from the bone marrow, can also contribute to tumor angiogenesis. Numerous molecules stimulate endothelial proliferation, migration and assembly. Formation of a vessel branch requires both migration and proliferation of endothelial cells. The molecules described in this application will inhibit proliferation and migration of endothelial cells through inhibition of exocytosis will stop the incorporation of receptors to the endothelial cell plasma membrane. Angiogenesis also depends on the survival of endothelial cells and this is supported by both autocrine and paracrine interactions in which pro-survival signals are secreted by endothelial cells, pericytes, and endothelial precursors. The molecules described in this application will inhibit both paracrine and autocrine loops by blocking exocytosis and the secretion of the pro-survival signals.

The ability of Clostridial toxins, such as, e.g., Botulinum neurotoxins (BoNTs), BoNT/A, BoNT/B, BoNT/C1, BoNT/D, BoNT/E, BoNT/F and BoNT/G, and Tetanus neurotoxin (TeNT), to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). Clostridial toxins commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea) BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc. San Francisco, Calif.). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder.

A Clostridial toxin treatment inhibits neurotransmitter release by disrupting the exocytotic process used to secret the neurotransmitter into the synaptic cleft. This disruption is ultimately accomplished by intracellular delivery of a Clostridial toxin light chain comprising an enzymatic domain where it cleaves a SNARE protein essential for the exocytotic process. There is a great desire by the pharmaceutical industry to expand the use of Clostridial toxin therapies beyond its current myo-relaxant applications to treat other ailments, such a s, e.g., various kinds of sensory nerve-based ailments like chronic pain, neurogenic inflammation and urogentital disorders, as well as non-nerve-based disorders, such as, e.g., pancreatitis and cancer. One approach that is currently being exploited to expand Clostridial toxin-based therapies involves modifying a Clostridial toxin so that the modified toxin has an altered cell targeting capability for a non-Clostridial toxin target cell. This re-targeted capability is achieved by replacing a naturally-occurring targeting domain of a Clostridial toxin with a targeting domain showing a selective binding activity for a non-Clostridial toxin receptor present in a non-Clostridial toxin target cell. Such modifications to a targeting domain result in a modified toxin that is able to selectively bind to a non-Clostridial toxin receptor (target receptor) present on a non-Clostridial toxin target cell (re-targeted). A modified Clostridial toxin with a targeting activity for a non-Clostridial toxin target cell can bind to a receptor present on the non-Clostridial toxin target cell, translocate into the cytoplasm, and exert its proteolytic effect on the SNARE complex of the non-Clostridial toxin target cell. In essence, a Clostridial toxin light chain comprising an enzymatic domain is intracellularly delivered to any desired cell by selecting the appropriate targeting domain.

The present specification discloses a class of modified Clostridial toxins retargeted to a non-Clostridial toxin receptor called Targeted Vesicular Exocytosis Modulating Proteins (TVEMPs), compositions comprising TVEMPs, and methods for treating an individual suffering from a cancer or a disease of hyperproliferation. A TVEMP is a recombinantly produced protein that comprises a targeting domain, and a translocation domain and enzymatic domain of a Clostridial toxin. The targeting is selected for its ability to bind to a receptor present on a target cancer cell of interest. The Clostridial toxin translocation domain and enzymatic domain serve to deliver the enzymatic domain into the cytoplasm of the target cell where it cleaves its cognate SNARE substrate. SNARE protein cleavage disrupts exocytosis, the process of cellular secretion or excretion in which substances contained in intracellular vesicles are discharged from the cell by fusion of the vesicular membrane with the outer cell membrane. This disruption prevents many fundamental processes of the cell, including, without limitation, insertion of transmembrane proteins including cell-surface receptors and signal transduction proteins; transportation of extracellular matrix proteins into the extracellular space; secretion of proteins including growth factors, angiogenic factors, neurotransmitters, hormones, and any other molecules involved in cellular communication; and expulsion of material including waste products, metabolites, and other unwanted or detrimental molecules. As such, exocytosis disruption severely affects cellular metabolism and ultimately cell viability. Thus a therapeutic molecule that reduces or inhibits exocytosis of a cell decreases the ability of a cell to divide and/or survive. Based on this premise, the TVEMPs disclosed herein are designed to target cells from a cancer or a disease of hyperproliferation or angiogenesis, where subsequent translocation of the enzymatic domain disrupts exocytosis by SNARE protein cleavage, thereby reducing the ability of cells from a cancer or a disease of hyperproliferation or endothelial cells in new vessels to survive or promote cellular overgrowth.

Thus, aspects of the present invention provide a composition comprising a TVEMP comprising a Prostate-Specific Membrane Antigen targeting domain, a Clostridial toxin translocation domain and a Clostridial toxin enzymatic domain. A TVEMP may further comprise an exogenous protease cleavage site. A TVEMP may be a pharmaceutical composition. Such a pharmaceutical composition may comprise, in addition to a TVEMP, a pharmaceutical carrier, a pharmaceutical component, or both.

Other aspects of the present invention provide a method of treating a prostate cancer in a mammal, the method comprising the step of administering to the mammal in need thereof a therapeutically effective amount of a composition including a TVEMP disclosed herein, wherein administration of the composition reduces a symptom associated with prostate cancer. The disclosed methods provide a safe, inexpensive, out patient-based treatment for the treatment of cancer.

Other aspects of the present invention provide a method of treating a benign prostatic hyperplasia in a mammal, the method comprising the step of administering to the mammal in need thereof a therapeutically effective amount of a composition including a TVEMP disclosed herein, wherein administration of the composition reduces a symptom associated with the benign prostatic hyperplasia. The disclosed methods provide a safe, inexpensive, out patient-based treatment for the treatment of benign prostatic hyperplasia.

Other aspects of the present invention provide a method of treating a cancer by reducing or inhibiting the neovascularization or angiogenesis associated with cancer in a mammal, the method comprising the step of administering to the mammal in need thereof a therapeutically effective amount of a composition including a TVEMP disclosed herein, wherein administration of the composition reduces a symptom associated with the neovascularization or angiogenesis associated with cancer. The disclosed methods provide a safe, inexpensive, out patient-based treatment for the treatment of neovascularization or angiogenesis associated with cancer.

Yet other aspects of the present invention provide a use of a TVEMP for the treatment of a prostate cancer in a mammal in need thereof, the use comprising the step of administering to the mammal a therapeutically effective amount of a composition including a TVEMP disclosed herein, wherein administration of the TVEMP reduces a symptom associated with the prostate cancer.

Yet other aspects of the present invention provide a use of a TVEMP for the treatment of a benign prostatic hyperplasia in a mammal in need thereof, the use comprising the step of administering to the mammal a therapeutically effective amount of a composition including a TVEMP disclosed herein, wherein administration of the TVEMP reduces a symptom associated with the benign prostatic hyperplasia.

Yet other aspects of the present invention provide a use of a TVEMP for the treatment of cancer by reducing or inhibiting the neovascularization or angiogenesis associated with cancer in a mammal in need thereof, the use comprising the step of administering to the mammal a therapeutically effective amount of a composition including a TVEMP disclosed herein, wherein administration of the TVEMP reduces a symptom associated with the neovascularization or angiogenesis associated with cancer.

Still other aspects of the present invention provide a use of a TVEMP in the manufacturing a medicament for treating a prostate cancer in a mammal in need thereof, wherein the TVEMP comprises a targeting domain, a Clostridial toxin translocation domain and a Clostridial toxin enzymatic domain. The TVEMP disclosed herein may further comprise an exogenous protease cleavage site. The TVEMP disclosed herein may be a pharmaceutical composition. Such a pharmaceutical composition may comprise, in addition to a TVEMP, a pharmaceutical carrier, a pharmaceutical component, or both.

Still other aspects of the present invention provide a use of a TVEMP in the manufacturing a medicament for treating a benign prostatic hyperplasia in a mammal in need thereof, wherein the TVEMP comprises a Prostate-Specific Membrane Antigen targeting domain, a Clostridial toxin translocation domain and a Clostridial toxin enzymatic domain. The TVEMP disclosed herein may further comprise an exogenous protease cleavage site. The TVEMP disclosed herein may be a pharmaceutical composition. Such a pharmaceutical composition may comprise, in addition to a TVEMP, a pharmaceutical carrier, a pharmaceutical component, or both.

Still other aspects of the present invention provide a use of a TVEMP in the manufacturing a medicament for treating of treating a cancer by reducing or inhibiting the neovascularization or angiogenesis associated with cancer in a mammal in need thereof, wherein the TVEMP comprises a targeting domain, a Clostridial toxin translocation domain and a Clostridial toxin enzymatic domain. The TVEMP disclosed herein may further comprise an exogenous protease cleavage site. The TVEMP disclosed herein may be a pharmaceutical composition. Such a pharmaceutical composition may comprise, in addition to a TVEMP, a pharmaceutical carrier, a pharmaceutical component, or both.

### BRIEF DESCRIPTION OF THE DRAWINGS

FIG. 1 shows a schematic of the current paradigm of neurotransmitter release and Clostridial toxin intoxication in a central and peripheral neuron. FIG. 1A shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 1B shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events are thought to occur, including, e.g., changes in the internal pH of the vesicle, formation of a channel pore comprising the HN domain of the Clostridial toxin heavy chain, separation of the Clostridial toxin light chain from the heavy chain, and release of the active light chain and 4) enzymatic target modification, where the activate light chain of Clostridial toxin proteolytically cleaves its target SNARE substrate, such as, e.g., SNAP-25, VAMP or Syntaxin, thereby preventing vesicle docking and neurotransmitter release.

FIG. 2 shows the domain organization of naturally-occurring Clostridial toxins. The single-chain form depicts the amino to carboxyl linear organization comprising an enzymatic domain, a translocation domain, and a targeting domain. The di-chain loop region located between the translocation and enzymatic domains is depicted by the double SS bracket. This region comprises an endogenous di-chain loop protease cleavage site that upon proteolytic cleavage with a naturally-occurring protease, such as, e.g., an endogenous Clostridial toxin protease or a naturally-occurring protease produced in the environment, converts the single-chain form of the toxin into the di-chain form. Above the single-chain form, the HCC region of the Clostridial toxin binding domain is depicted. This region comprises the β-trefoil domain which comprises in an amino to carboxyl linear organization an α-fold, a β4/β5 hairpin turn, a β-fold, a β8/β9 hairpin turn and a γ-fold.

FIG. 3 shows TVEMPs with a targeting domain located at the amino terminus. FIG. 3A depicts the single-chain polypeptide form of a TVEMP with an amino to carboxyl linear organization comprising a targeting domain, a translocation domain, a di-chain loop region comprising an exogenous protease cleavage site (P), and an enzymatic domain. Upon proteolytic cleavage with a P protease, the single-chain form of the toxin is converted to the di-chain form. FIG. 3B depicts the single polypeptide form of a TVEMP with an amino to carboxyl linear organization comprising a targeting domain, an enzymatic domain, a di-chain loop region comprising an exogenous protease cleavage site (P), and a translocation domain. Upon proteolytic cleavage with a P protease, the single-chain form of the toxin is converted to the di-chain form.

FIG. 4 shows TVEMPs with a targeting domain located between the other two domains. FIG. 4A depicts the single polypeptide form of a TVEMP with an amino to carboxyl linear organization comprising an enzymatic domain, a di-chain loop region comprising an exogenous protease cleavage site (P), a targeting domain, and a translocation domain. Upon proteolytic cleavage with a P protease, the single-chain form of the toxin is converted to the di-chain form. FIG. 4B depicts the single polypeptide form of a TVEMP with an amino to carboxyl linear organization comprising a translocation domain, a di-chain loop region comprising an exogenous protease cleavage site (P), a targeting domain, and an enzymatic domain. Upon proteolytic cleavage with a P protease, the single-chain form of the toxin is converted to the di-chain form. FIG. 4C depicts the single polypeptide form of a TVEMP with an amino to carboxyl linear organization comprising an enzymatic domain, a targeting domain, a di-chain loop region comprising an exogenous protease cleavage site (P), and a translocation domain. Upon proteolytic cleavage with a P protease, the single-chain form of the toxin is converted to the di-chain form. FIG. 4D depicts the single polypeptide form of a TVEMP with an amino to carboxyl linear organization comprising a translocation domain, a targeting domain, a di-chain loop region comprising an exogenous protease cleavage site (P), and an enzymatic domain. Upon proteolytic cleavage with a P protease, the single-chain form of the toxin is converted to the di-chain form.

FIG. 5 shows TVEMPs with a targeting domain located at the carboxyl terminus. FIG. 5A depicts the single polypeptide form of a TVEMP with an amino to carboxyl linear organization comprising an enzymatic domain, a di-chain loop region comprising an exogenous protease cleavage site (P), a translocation domain, and a targeting domain. Upon proteolytic cleavage with a P protease, the single-chain form of the toxin is converted to the di-chain form. FIG. 5B depicts the single polypeptide form of a TVEMP with an amino to carboxyl linear organization comprising a translocation domain, a di-chain loop region comprising an exogenous protease cleavage site (P), an enzymatic domain, and a targeting domain. Upon proteolytic cleavage with a P protease, the single-chain form of the toxin is converted to the di-chain form.

### DETAILED DESCRIPTION

Cancer refers to the uncontrolled growth of cells in a mammalian body, and as such is fundamentally a disease that affects the regulatory mechanism the body uses to control cell growth. In order for a normal cell to transform into a cancer cell, genes which regulate cell growth and differentiation must be altered. Genetic changes can occur at many levels, from gain or loss of entire chromosomes to a mutation affecting a single DNA nucleotide. The vast catalog of cancer cell genotypes is a manifestation of six essential alterations in cell physiology that collectively dictate malignant growth: 1) self-sufficiency in growth signals; 2) insensitivity to growth-inhibitory (antigrowth) signals; 3) evasion of programmed cell death (apoptosis); 4) limitless replicative potential; 5) sustained angiogenesis; and 6) tissue invasion and metastasis. Hanahan and Weinberg, The Hallmarks of Cancer, Cell 100(1): 57-70 (2000).

One way cancer cells exhibit self-sufficiency in growth signals is by the expression of oncogenes. Oncogenes may be normal genes which are expressed at inappropriately high levels, or altered genes which have novel properties. In either case, expression of these genes promote the malignant phenotype of cell growth exhibited by cancer cells through a variety of ways. Many can produce secreted factors between cells, like hormones, which encourage mitosis, the effect of which depends on the signal transduction of the receiving tissue or cells. Thus, when a hormone receptor on a recipient cell is stimulated, the signal is conducted from the surface of the cell to the cell nucleus to effect some change in gene transcription regulation at the nuclear level. Some oncogenes are part of the signal transduction system itself, or the signal receptors in cells and tissues themselves, thus controlling the sensitivity to such hormones. Oncogenes often produce mitogens, or are involved in transcription of DNA in protein synthesis, which creates the proteins and enzymes responsible for producing the products and biochemicals cells use and interact with. Mutations in proto-oncogenes, which are the normally quiescent counterparts of oncogenes, can modify their expression and function, increasing the amount or activity of the product protein. When this happens, the proto-oncogenes become oncogenes, and this transition upsets the normal balance of cell cycle regulation in the cell, making uncontrolled growth possible. The chance of cancer cannot be reduced by removing proto-oncogenes from the genome, even if this were possible, as they are critical for growth, repair and homeostasis of the organism. It is only when they become mutated that the signals for growth become excessive. Therefore, therapeutic strategies to inhibit cell growth signals in cancer cells have the potential to provide powerful tools to treat cancers exhibiting self-sufficiency in growth signals due to oncogene expression. Moreover, many cancer cells express growth factor receptors and the ligands that activate those receptors (autocrine loops). In normal tissue one type of cell expresses the growth factor receptor and another type the ligand (paracrine loops) in an effort to maintain homeostasis. Cancer cells by expressing ligand and receptor acquire self-sufficiency for growth.

One way that cancer cells display an insensitivity to growth-inhibitory (antigrowth) signals is by the inhibition of expression of tumor suppressor genes. Tumor suppressor genes are genes which inhibit cell division, survival, or other properties of cancer cells. Tumor suppressor genes are often disabled by cancer-promoting genetic changes. Typically, changes in many genes are required to transform a normal cell into a cancer cell. Generally, tumor suppressors are transcription factors that are activated by cellular stress or DNA damage. Often DNA damage will cause the presence of free-floating genetic material as well as other signs, and will trigger enzymes and pathways which lead to the activation of tumor suppressor genes. The functions of such genes is to arrest the progression of the cell cycle in order to carry out DNA repair, preventing mutations from being passed on to daughter cells. Therefore, therapeutic strategies to inhibit cell division signals in cancer cells have the potential to provide powerful tools to treat cancers displaying insensitivity to growth-inhibitory signals due to the suppression of tumor suppressor gene expression.

One way that cancer cells evade programmed cell death (apoptosis) is by continuous exposure to cell survival signals (antiapoptotic signals). Signals to induce cell survival or cell death are provided by sensors in the plasma membrane (i.e. death receptors) and by intracellular sensors Intracellular sensors monitor the cell's health and in response to detecting abnormalities like DNA damage, oncogene action, survival factor insufficiency, or hypoxia, they activate the death pathway. Therefore, cancer cells should undergo apoptosis as they have DNA damage, activated oncogene, or hypoxia in the center of the tumor. Several types of cancer cells are dependent on survival signals delivered by autocrine loops to counteract apoptotic signals triggered by DNA damage present in these cells. These autocrine loops are established by cancer cells through the expression of growth factor ligands and their cognate receptors. Therefore, therapeutic strategies to inhibit the reception of cell survival signals by cancer cells have the potential to provide powerful tools to treat cancers with overactivation of antiapoptotic signals. In fact, there is evidence in the literature that hormone and/or growth factor withdraw can produce apoptosis in cancer cells as the balance between survival and apoptotic signals is restored.

Another acquired capability of cancer cells is the limitless replicative potential of the tumor cells. Cancer cells overcome the limits of proliferation by maintaining integrity of the telomeres and avoiding the crisis state that results from continue multiplication that erodes the telomeres. Cancer cells overexpress the enzyme telomerase that maintains the size of the telomeres and allow for limitless replicative potential. But another important step is the ability to deliver membrane to the plasma membrane to complete the mitotic process.

As cells proliferate within a tumor they also face other challenges like the limited supply of oxygen and nutrients that would induce apoptosis. So to be able to sustain growth and proliferation the tumor needs to encourage the growth of existing blood vessels as well as the growth of new blood vessels, a process highly regulated in mature tissues. Cancer cells secrete pro-angiogenic factors to activate receptors in endothelial cells. In addition, pro-angiogenic factors sequestered in the extracellular matrix can be released by digestion of the matrix performed by proteases secreted by tumor cells. Inhibition of angiogenesis is a validated therapeutic target as several approved drugs target this pathway as a treatment for cancer and other pro-angiogenesis diseases.

Finally, tumor cells acquire the capability to invade adjacent tissues and metastasize to distant sites. To accomplish that, tumor cells may first be able to change their adhesion capabilities by altering the expression of adhesion proteins and integrins. More importantly, to be able to migrate cancer cells need to be able to degrade the extracellular matrix that surround them. Cancer cells overexpress matrix degrading proteases either as secreted factors or as membrane anchored proteases and downregulate the expression of protease inhibitors.

As uncontrolled cell growth is the underlying cause of all cancers, compounds and methods that can reduce or prevent this uncontrolled cell growth would be an effective treatment for cancer. The present specification discloses compounds and methods that can reduce or prevent the uncontrolled cell growth displayed by cancer cells. The novel retargeted endopeptidases comprise, in part, a binding domain and an enzymatic domain. The binding domain directs the retargeted endopeptidase to a specific cancer cell type that is expressing the cognate receptor for the binding domain. The endopeptidase activity of the enzymatic domain inhibits exocytosis by cleaving the appropriate target SNARE protein, thereby disrupting exocytosis and delivery of receptors and membrane to the plasma membrane. Preventing exocytosis in cancers cells is therapeutically useful because disruption would, e.g., 1) prevent the release of secreting growth factors by cancer cells which encourage mitosis; or 2) prevent delivery of receptors to the plasma membrane of cancer cells which would interfere with the cancer cell's ability to receive cancer-promoting signals, such as, e.g., receiving a growth stimulating signal or a cell survival signal. The later would be useful in eliminating cancer cells by tilting the balance towards apoptosis of the cancer cells; 3) prevent delivery of membrane to the plasma membrane and thus stopping the process of mitosis that can only occur with a net gain of membrane to produce daughter cells; 4) reduce angiogenesis by inhibiting the release of pro-angiogenic factors by tumor cells or the extracellular matrix; 5) inhibit invasion and metastasis by inhibiting the release of proteases and by interfering with the switch of adhesion proteins and integrins.

Thus, while current cancer therapeutics in the market target only one pathway at a time and are therefore only partially effective and allow cancer cells to acquire resistance to the treatment, a TVEMP-based therapy by means of inhibition of exocytosis, receptor delivery, and membrane delivery, will target several pathways with a single drug delivering a stronger punch to tumor cells and therefore being more effective. Moreover, as normal cells are not proliferating and are not so dependent on survival signals they would not be affected by the therapy.

In a similar manner, the stroma and epithelial cell hyperproliferation seen in BPH can be effectively treated using the TVEMPs and methods disclosed herein. The observed increase in secretion of cytokines and growth factors in BPH is dependent on a SNARE-mediated exocytotic process. The TVEMPs disclosed herein target the cells aberrantly secreting these factors and inhibit this process. This exocytotic inhibition reduces or eliminates the secretion of growth factors and cytokines, thereby removing the signals promoting this hyperproliferation. Moreover, the histopathology of BPH strongly implicates local paracrine and autocrine growth factors and inflammatory cytokines loops in its pathogenesis, needing the presence of the appropriate receptor in the surface of hyperproliferating cells to receive the signal and transducer the message to the cell nucleus. The inhibition of exocytosis produced by SNARE cleavage will prevent delivery of receptors to the plasma membrane of hyperproliferating cells which would interfere with the cell's ability to receive hyperproliferation-promoting signals, such as, e.g., receiving a growth stimulating signal or a cell survival signal. The later would be useful in eliminating hyperproliferating cells by tilting the balance towards apoptosis and growth arrest.

In pathological states such as tumor growth, there is an imbalance between endogenous stimulator and inhibitor levels, leading to an “angiogenic switch”. Tumors require a vascular supply to grow. Without blood vessels, tumors cannot grow beyond a critical size or metastasize to another organ and blocking angiogenesis can suppress tumor growth. Formation of a vessel branch requires both migration and proliferation of endothelial cells. The TVEMP molecules described in this application will inhibit proliferation and migration of endothelial cells by delivering a Clostridial toxin enzymatic domain that through inhibition of exocytosis will stop the incorporation of receptors to the endothelial cell plasma membrane. By inhibiting receptor delivery the endothelial cells cannot bind pro-angiogenic molecules secreted by other cells in the environment (i.e. tumor cells, inflammatory cells, dendritic cells, etc. . . . ) or pro-angiogenic molecules present in the extracellular matrix. The lack of signaling will make the endothelial cells to stop proliferating and migrating becoming quiescent. In contrast to strategies that target a single receptor (i.e. VEGFR), the proposed TVEMPs will affect all the receptors in the target cells that are delivered to the plasma membrane by SNAREs. Formation of a vessel branch requires both migration and proliferation of endothelial cells. The TVEMP molecules described in this application will inhibit proliferation and migration of endothelial cells by delivering a BoNT-LC that through inhibition of exocytosis will stop the incorporation of receptors to the endothelial cell plasma membrane. By inhibiting receptor delivery the endothelial cells cannot bind pro-angiogenic molecules secreted by other cells in the environment (i.e. tumor cells, inflammatory cells, dendritic cells, etc. . . . ) or pro-angiogenic molecules present in the extracellular matrix. The lack of signaling will make the endothelial cells to stop proliferating and migrating becoming quiescent. In contrast to strategies that target a single receptor (i.e. VEGFR), the proposed TVEMPs will affect all the receptors in the target cells that are delivered to the plasma membrane by SNAREs.

Aspects of the present invention provide, in part, a TVEMP. As used herein, a “TVEMP” means any molecule comprising a targeting domain, a Clostridial toxin translocation domain and a Clostridial toxin enzymatic domain. Exemplary TVEMPs useful to practice aspects of the present invention are disclosed in, e.g., Steward, L. E. et al., Activatable Clostridial Toxins, U.S. Pat. No. 7,740,868; Dolly, J. O. et al., Activatable Clostridial Neurotoxins, U.S. Pat. No. 7,709,228; Steward, L. E. et al., Activatable Clostridial Toxins, U.S. Patent Publication 2009/0069238; Foster, K. A. et al., Fusion Proteins, US Patent Publication 2009/0035822; and Foster, K. A. et al., Non-Cytotoxic Protein Conjugates, US Patent Publication 2008/0187960; Steward, L. E. et al., Activatable Clostridial Toxins, U.S. Patent Publication 2008/0161226, each of which is incorporated by reference in its entirety.

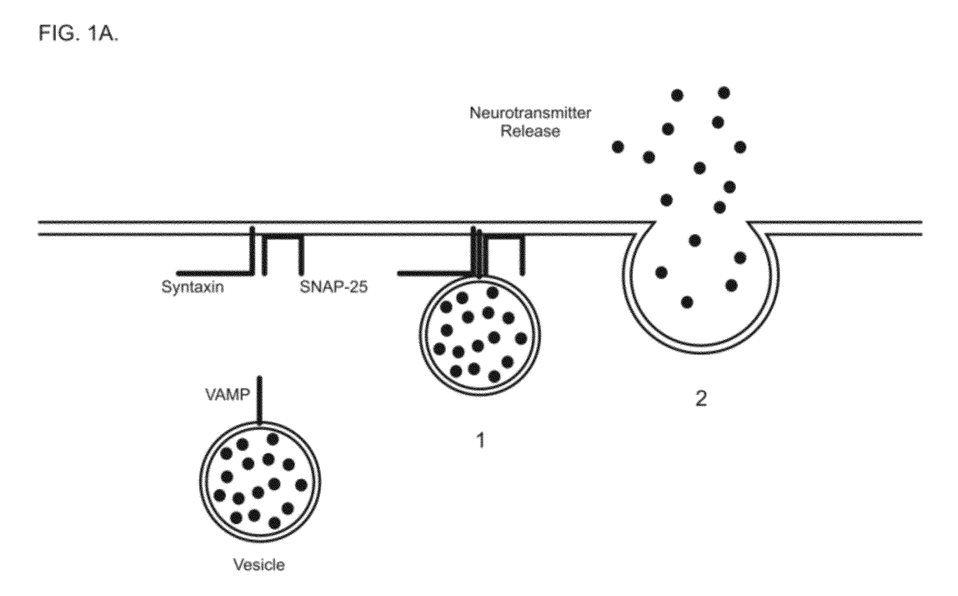

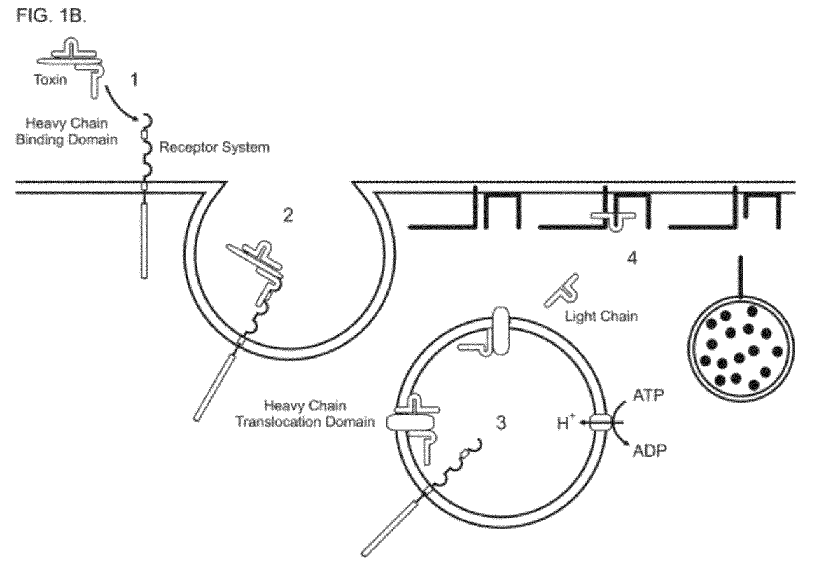

Clostridial toxins are each translated as a single chain polypeptide of approximately 150 kDa that is subsequently cleaved by proteolytic scission within a disulfide loop by a naturally-occurring protease (FIG. 1). This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains. The naturally-occurring protease used to convert the single chain molecule into the di-chain is currently not known. In some serotypes, such as, e.g., BoNT/A, the naturally-occurring protease is produced endogenously by the bacteria serotype and cleavage occurs within the cell before the toxin is release into the environment. However, in other serotypes, such as, e.g., BoNT/E, the bacterial strain appears not to produce an endogenous protease capable of converting the single chain form of the toxin into the di-chain form. In these situations, the toxin is released from the cell as a single-chain toxin which is subsequently converted into the di-chain form by a naturally-occurring protease found in the environment.

Each mature di-chain molecule comprises three functionally distinct domains: 1) an enzymatic domain located in the LC that includes a metalloprotease region containing a zinc-dependent endopeptidase activity which specifically targets core components of the neurotransmitter release apparatus; 2) a translocation domain contained within the amino-terminal half of the HC (H_{N}) that facilitates release of the LC from intracellular vesicles into the cytoplasm of the target cell; and 3) a binding domain found within the carboxyl-terminal half of the HC (H_{C}) that determines the binding activity and binding specificity of the toxin to the receptor complex located at the surface of the target cell. D. B. Lacy and R. C. Stevens, Sequence Homology and Structural Analysis of the Clostridial Neurotoxins, J. Mol. Biol. 291: 1091-1104 (1999). The H_{C} domain comprises two distinct structural features of roughly equal size, separated by an α-helix, designated the H_{CN} and H_{CC} subdomains. Table 1 gives approximate boundary regions for each domain and subdomain found in exemplary Clostridial toxins.

TABLE 1
Clostridial Toxin Reference Sequences and Regions

| | SEQ ID | | Di-Chain | | H_{C} | | |
|---|---|---|---|---|---|---|---|
| Toxin | NO: | LC | Loop | H_{N} | H_{CN} | α-Linker | H_{CC} |
| BoNT/A | 1 | M1/P2-L429 | C430-C454 | I455-I873 | I874-N1080 | E1081-Q1091 | S1092-L1296 |
| BoNT/B | 6 | M1/P2-M436 | C437-C446 | I447-I860 | L861-S1067 | Q1068-Q1078 | S1079-E1291 |
| BoNT/C1 | 11 | M1/P2-F436 | C437-C453 | R454-I868 | N869-D1081 | G1082-L1092 | Q1093-E1291 |
| BoNT/D | 13 | M1/T2-V436 | C437-C450 | I451-I864 | N865-S1069 | N1069-Q1079 | I1080-E1276 |
| BoNT/E | 15 | M1/P2-F411 | C412-C426 | I427-I847 | K848-D1055 | E1056-E1066 | P1067-K1252 |
| BoNT/F | 18 | M1/P2-F428 | C429-C445 | I446-I865 | K866-D1075 | K1076-E1086 | P1087-E1274 |
| BoNT/G | 21 | M1/P2-M435 | C436-C450 | I451-I865 | S866-N1075 | A1076-Q1086 | S1087-E1297 |
| TeNT | 22 | M1/P2-L438 | C439-C467 | I468-L881 | K882-N1097 | P1098-Y1108 | L1109-D1315 |
| BaNT | 23 | M1/P2-L420 | C421-C435 | I436-I857 | I858-D1064 | K1065-E1075 | P1076-E1268 |
| BuNT | 24 | M1/P2-F411 | C412-C426 | I427-I847 | K848-D1055 | E1056-E1066 | P1067-K1251 |

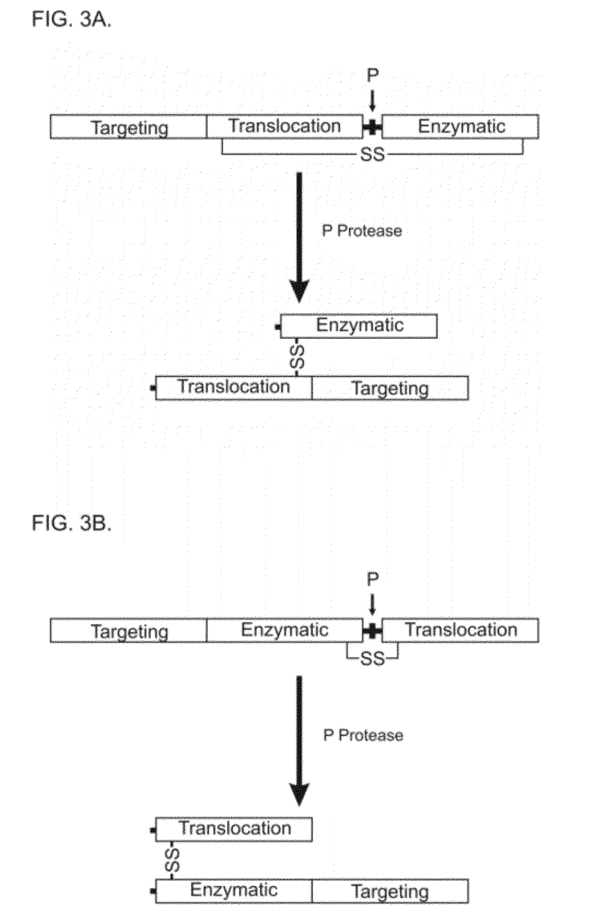

The binding, translocation, and enzymatic activity of these three functional domains are all necessary for toxicity. While all details of this process are not yet precisely known, the overall cellular intoxication mechanism whereby Clostridial toxins enter a neuron and inhibit neurotransmitter release is similar, regardless of serotype or subtype. Although the applicants have no wish to be limited by the following description, the intoxication mechanism can be described as comprising at least four steps: 1) receptor binding, 2) complex internalization, 3) light chain translocation, and 4) enzymatic target modification (FIG. 3). The process is initiated when the H_{C} domain of a Clostridial toxin binds to a toxin-specific receptor system located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the toxin/receptor complexes are internalized by endocytosis and the internalized vesicles are sorted to specific intracellular routes. The translocation step appears to be triggered by the acidification of the vesicle compartment. This process seems to initiate two important pH-dependent structural rearrangements that increase hydrophobicity and promote formation di-chain form of the toxin. Once activated, light chain endopeptidase of the toxin is released from the intracellular vesicle into the cytosol where it appears to specifically target one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface. The selective proteolysis of synaptic SNAREs accounts for the block of neurotransmitter release caused by Clostridial toxins in vivo. The SNARE protein targets of Clostridial toxins are common to exocytosis in a variety of non-neuronal types; in these cells, as in neurons, light chain peptidase activity inhibits exocytosis, see, e.g., Yann Humeau et al., How Botulinum and Tetanus Neurotoxins Block Neurotransmitter Release, 82(5) Biochimie. 427-446 (2000); Kathryn Turton et al., Botulinum and Tetanus Neurotoxins: Structure, Function and Therapeutic Utility, 27(11) Trends Biochem. Sci. 552-558. (2002); Giovanna Lalli et al., The Journey of Tetanus and Botulinum Neurotoxins in Neurons, 11(9) Trends Microbiol. 431-437, (2003).

Aspects of the present specification provide, in part, a TVEMP comprising a Clostridial toxin enzymatic domain. As used herein, the term “Clostridial toxin enzymatic domain” refers to any Clostridial toxin polypeptide that can execute the enzymatic target modification step of the intoxication process. Thus, a Clostridial toxin enzymatic domain specifically targets a Clostridial toxin substrate and encompasses the proteolytic cleavage of a Clostridial toxin substrate, such as, e.g., SNARE proteins like a SNAP-25 substrate, a VAMP substrate, and a Syntaxin substrate. Non-limiting examples of a Clostridial toxin enzymatic domain include, e.g., a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, and a BuNT enzymatic domain.

A Clostridial toxin enzymatic domain includes, without limitation, naturally occurring Clostridial toxin enzymatic domain variants, such as, e.g., Clostridial toxin enzymatic domain isoforms and Clostridial toxin enzymatic domain subtypes; and non-naturally occurring Clostridial toxin enzymatic domain variants, such as, e.g., conservative Clostridial toxin enzymatic domain variants, non-conservative Clostridial toxin enzymatic domain variants, active Clostridial toxin enzymatic domain fragments thereof, or any combination thereof.

As used herein, the term “Clostridial toxin enzymatic domain variant,” whether naturally-occurring or non-naturally-occurring, refers to a Clostridial toxin enzymatic domain that has at least one amino acid change from the corresponding region of the disclosed reference sequences (Table 1) and can be described in percent identity to the corresponding region of that reference sequence. Unless expressly indicated, Clostridial toxin enzymatic domain variants useful to practice disclosed embodiments are variants that execute the enzymatic target modification step of the intoxication process. As non-limiting examples, a BoNT/A enzymatic domain variant will have at least one amino acid difference, such as, e.g., an amino acid substitution, deletion or addition, as compared to amino acids 1/2-429 of SEQ ID NO: 1; a BoNT/B enzymatic domain variant will have at least one amino acid difference, such as, e.g., an amino acid substitution, deletion or addition, as compared to amino acids 1/2-436 of SEQ ID NO: 6; a BoNT/C1 enzymatic domain variant will have at least one amino acid difference, such as, e.g., an amino acid substitution, deletion or addition, as compared to amino acids 1/2-436 of SEQ ID NO: 11; a BoNT/D enzymatic domain variant will have at least one amino acid difference, such as, e.g., an amino acid substitution, deletion or addition, as compared to amino acids 1/2-436 of SEQ ID NO: 13; a BoNT/E enzymatic domain variant will have at least one amino acid difference, such as, e.g., an amino acid substitution, deletion or addition, as compared to amino acids 1/2-411 of SEQ ID NO: 15; a BoNT/F enzymatic domain variant will have at least one amino acid difference, such as, e.g., an amino acid substitution, deletion or addition, as compared to amino acids 1/2-428 of SEQ ID NO: 18; a BoNT/G enzymatic domain variant will have at least one amino acid difference, such as, e.g., an amino acid substitution, deletion or addition, as compared to amino acids 1/2-438 of SEQ ID NO: 21; a TeNT enzymatic domain variant will have at least one amino acid difference, such as, e.g., an amino acid substitution, deletion or addition, as compared to amino acids 1/2-438 of SEQ ID NO: 22; a BaNT enzymatic domain variant will have at least one amino acid difference, such as, e.g., an amino acid substitution, deletion or addition, as compared to amino acids 1/2-420 of SEQ ID NO: 23; and a BuNT enzymatic domain variant will have at least one amino acid difference, such as, e.g., an amino acid substitution, deletion or addition, as compared to amino acids 1/2-411 of SEQ ID NO: 24.

It is recognized by those of skill in the art that within each serotype of Clostridial toxin there can be naturally occurring Clostridial toxin enzymatic domain variants that differ somewhat in their amino acid sequence, and also in the nucleic acids encoding these proteins. For example, there are presently five BoNT/A subtypes, BoNT/A1, BoNT/A2, BoNT/A3, BoNT/A4, and BoNT/A5, with specific enzymatic domain subtypes showing about 80% to 95% amino acid identity when compared to the BoNT/A enzymatic domain of SEQ ID NO: 1. As used herein, the term “naturally occurring Clostridial toxin enzymatic domain variant” refers to any Clostridial toxin enzymatic domain produced by a naturally-occurring process, including, without limitation, Clostridial toxin enzymatic domain isoforms produced from alternatively-spliced transcripts, Clostridial toxin enzymatic domain isoforms produced by spontaneous mutation and Clostridial toxin enzymatic domain subtypes. A naturally occurring Clostridial toxin enzymatic domain variant can function in substantially the same manner as the reference Clostridial toxin enzymatic domain on which the naturally occurring Clostridial toxin enzymatic domain variant is based, and can be substituted for the reference Clostridial toxin enzymatic domain in any aspect of the present specification.

A non-limiting examples of a naturally occurring Clostridial toxin enzymatic domain variant is a Clostridial toxin enzymatic domain isoform such as, e.g., a BoNT/A enzymatic domain isoform, a BoNT/B enzymatic domain isoform, a BoNT/C1 enzymatic domain isoform, a BoNT/D enzymatic domain isoform, a BoNT/E enzymatic domain isoform, a BoNT/F enzymatic domain isoform, a BoNT/G enzymatic domain isoform, a TeNT enzymatic domain isoform, a BaNT enzymatic domain isoform, and a BuNT enzymatic domain isoform. Another non-limiting examples of a naturally occurring Clostridial toxin enzymatic domain variant is a Clostridial toxin enzymatic domain subtype such as, e.g., an enzymatic domain from subtype BoNT/A1, BoNT/A2, BoNT/A3, BoNT/A4, or BoNT/A5; an enzymatic domain from subtype BoNT/B1, BoNT/B2, BoNT/Bbv, or BoNT/Bnp; an enzymatic domain from subtype BoNT/C1-1 or BoNT/C1-2; an enzymatic domain from subtype BoNT/E1, BoNT/E2 and BoNT/E3; an enzymatic domain from subtype BoNT/F1, BoNT/F2, or BoNT/F3; and an enzymatic domain from subtype BuNT-1 or BuNT-2.

As used herein, the term “non-naturally occurring Clostridial toxin enzymatic domain variant” refers to any Clostridial toxin enzymatic domain produced with the aid of human manipulation, including, without limitation, Clostridial toxin enzymatic domains produced by genetic engineering using random mutagenesis or rational design and Clostridial toxin enzymatic domains produced by chemical synthesis. Non-limiting examples of non-naturally occurring Clostridial toxin enzymatic domain variants include, e.g., conservative Clostridial toxin enzymatic domain variants, non-conservative Clostridial toxin enzymatic domain variants, Clostridial toxin enzymatic domain chimeric variants, and active Clostridial toxin enzymatic domain fragments.

As used herein, the term “conservative Clostridial toxin enzymatic domain variant” refers to a Clostridial toxin enzymatic domain that has at least one amino acid substituted by another amino acid or an amino acid analog that has at least one property similar to that of the original amino acid from the reference Clostridial toxin enzymatic domain sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin enzymatic domain variant can function in substantially the same manner as the reference Clostridial toxin enzymatic domain on which the conservative Clostridial toxin enzymatic domain variant is based, and can be substituted for the reference Clostridial toxin enzymatic domain in any aspect of the present specification. Non-limiting examples of a conservative Clostridial toxin enzymatic domain variant include, e.g., conservative BoNT/A enzymatic domain variants, conservative BoNT/B enzymatic domain variants, conservative BoNT/C1 enzymatic domain variants, conservative BoNT/D enzymatic domain variants, conservative BoNT/E enzymatic domain variants, conservative BoNT/F enzymatic domain variants, conservative BoNT/G enzymatic domain variants, conservative TeNT enzymatic domain variants, conservative BaNT enzymatic domain variants, and conservative BuNT enzymatic domain variants.

As used herein, the term “non-conservative Clostridial toxin enzymatic domain variant” refers to a Clostridial toxin enzymatic domain in which 1) at least one amino acid is deleted from the reference Clostridial toxin enzymatic domain on which the non-conservative Clostridial toxin enzymatic domain variant is based; 2) at least one amino acid added to the reference Clostridial toxin enzymatic domain on which the non-conservative Clostridial toxin enzymatic domain is based; or 3) at least one amino acid is substituted by another amino acid or an amino acid analog that does not share any property similar to that of the original amino acid from the reference Clostridial toxin enzymatic domain sequence (Table 1). A non-conservative Clostridial toxin enzymatic domain variant can function in substantially the same manner as the reference Clostridial toxin enzymatic domain on which the non-conservative Clostridial toxin enzymatic domain variant is based, and can be substituted for the reference Clostridial toxin enzymatic domain in any aspect of the present specification. Non-limiting examples of a non-conservative Clostridial toxin enzymatic domain variant include, e.g., non-conservative BoNT/A enzymatic domain variants, non-conservative BoNT/B enzymatic domain variants, non-conservative BoNT/C1 enzymatic domain variants, non-conservative BoNT/D enzymatic domain variants, non-conservative BoNT/E enzymatic domain variants, non-conservative BoNT/F enzymatic domain variants, non-conservative BoNT/G enzymatic domain variants, and non-conservative TeNT enzymatic domain variants, non-conservative BaNT enzymatic domain variants, and non-conservative BuNT enzymatic domain variants.

As used herein, the term “active Clostridial toxin enzymatic domain fragment” refers to any of a variety of Clostridial toxin fragments comprising the enzymatic domain can be useful in aspects of the present specification with the proviso that these enzymatic domain fragments can specifically target the core components of the neurotransmitter release apparatus and thus participate in executing the overall cellular mechanism whereby a Clostridial toxin proteolytically cleaves a substrate. The enzymatic domains of Clostridial toxins are approximately 420-460 amino acids in length and comprise an enzymatic domain (Table 1). Research has shown that the entire length of a Clostridial toxin enzymatic domain is not necessary for the enzymatic activity of the enzymatic domain. As a non-limiting example, the first eight amino acids of the BoNT/A enzymatic domain are not required for enzymatic activity. As another non-limiting example, the first eight amino acids of the TeNT enzymatic domain are not required for enzymatic activity. Likewise, the carboxyl-terminus of the enzymatic domain is not necessary for activity. As a non-limiting example, the last 32 amino acids of the BoNT/A enzymatic domain are not required for enzymatic activity. As another non-limiting example, the last 31 amino acids of the TeNT enzymatic domain are not required for enzymatic activity. Thus, aspects of this embodiment include Clostridial toxin enzymatic domains comprising an enzymatic domain having a length of, e.g., at least 350, 375, 400, 425, or 450 amino acids. Other aspects of this embodiment include Clostridial toxin enzymatic domains comprising an enzymatic domain having a length of, e.g., at most 350, 375, 400, 425, or 450 amino acids.

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art and from the teaching herein.

Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein Sequence Alignments by Iterative Refinement as Assessed by Reference to Structural Alignments, 264(4) J. Mol. Biol. 823-838 (1996).

Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics,:1428-1435 (2004).

Hybrid methods combine functional aspects of both global and local alignment methods. Non-limiting methods include, e.g., segment-to-segment comparison, see, e.g., Burkhard Morgenstern et al., Multiple DNA and Protein Sequence Alignment Based On Segment-To-Segment Comparison, 93(22) Proc. Natl. Acad. Sci. U.S.A. 12098-12103 (1996); T-Coffee, see, e.g., Cédric Notredame et al., T-Coffee: A Novel Algorithm for Multiple Sequence Alignment, 302(1) J. Mol. Biol. 205-217 (2000); MUSCLE, see, e.g., Robert C. Edgar, MUSCLE: Multiple Sequence Alignment With High Score Accuracy and High Throughput, 32(5) Nucleic Acids Res. 1792-1797 (2004); and DIALIGN-T, see, e.g., Amarendran R Subramanian et al., DIALIGN-T: An Improved Algorithm for Segment-Based Multiple Sequence Alignment, 6(1) BMC Bioinformatics 66 (2005).

The present specification describes various polypeptide variants where one amino acid is substituted for another, such as, e.g., Clostridial toxin enzymatic domain variants, Clostridial toxin translocation domain variants, targeting domain variants, and protease cleavage site variants, A substitution can be assessed by a variety of factors, such as, e.g., the physic properties of the amino acid being substituted (Table 2) or how the original amino acid would tolerate a substitution (Table 3). The selections of which amino acid can be substituted for another amino acid in a polypeptide are known to a person of ordinary skill in the art.

TABLE 2
Amino Acid Properties

| Property | Amino Acids |
|---|---|
| Aliphatic | G, A, I, L, M, P, V |
| Aromatic | F, H, W, Y |
| C-beta branched | I, V, T |
| Hydrophobic | C, F, I, L, M, V, W |
| Small polar | D, N, P |
| Small non-polar | A, C, G, S, T |
| Large polar | E, H, K, Q, R, W, Y |
| Large non-polar | F, I, L, M, V |
| Charged | D, E, H, K, R |
| Uncharged | C, S, T |
| Negative | D, E |
| Positive | H, K, R |
| Acidic | D, E |
| Basic | K, R |
| Amide | N, Q |

TABLE 3
Amino Acid Substitutions

| Amino Acid | Favored Substitution | Neutral Substitutions | Disfavored substitution |
|---|---|---|---|
| A | G, S, T | C, E, I, K, M, L, P, Q, R, V | D, F, H, N, Y, W |
| C | F, S, Y, W | A, H, I, M, L, T, V | D, E, G, K, N, P, Q, R |
| D | E, N | G, H, K, P, Q, R, S, T | A, C, I, L, |
| E | D, K, Q | A, H, N, P, R, S, T | C, F, G, I, L, M, V, W, Y |
| F | M, L, W, Y | C, I, V | A, D, E, G, H, K, N, P, Q, R, S, T |
| G | A, S | D, K, N, P, Q, R | C, E, F, H, I, L, M, T, V, W, Y |
| H | N, Y | C, D, E, K, Q, R, S, T, W | A, F, G, I, L, M, P, V |
| I | V, L, M | A, C, T, F, Y | D, E, G, H, K, N, P, Q, R, S, W |
| K | Q, E, R | A, D, G, H, M, N, P, S, T | C, F, I, L, V, W, Y |
| L | F, I, M, V | A, C, W, Y | D, E, G, H, K, N, P, Q, R, S, T |
| M | F, I, L, V | A, C, R, Q, K, T, W, Y | D, E, G, H, N, P, S |
| N | D, H, S | E, G, K, Q, R, T | A, C, F, I, L, M, P, V, W, Y |
| P | — | A, D, E, G, K, Q, R, S, T | C, F, H, I, L, M, N, V, W, Y |
| Q | E, K, R | A, D, G, H, M, N, P, S, T | C, F, I, L, V, W, Y |
| R | K, Q | A, D, E, G, H, M, N, P, S, T | C, F, I, L, V, W, Y |
| S | A, N, T | C, D, E, G, H, K, P, Q, R, T | F, I, L, M, V, W, Y |
| T | S | A, C, D, E, H, I, K, M, N, P, Q, R, V | F, G, L, W, Y |
| V | I, L, M | A, C, F, T, Y | D, E, G, H, K, N, P, Q, R, S, W |
| W | F, Y | H, L, M | A, C, D, E, G, I, K, N, P, Q, R, S, T, V |
| Y | F, H, W | C, I, L, M, V | A, D, E, G, K, N, P, Q, R, S, T |

Matthew J. Betts and Robert, B. Russell, Amino Acid Properties and Consequences of Substitutions, pp. 289-316, In Bioinformatics for Geneticists, (eds Michael R. Barnes, Ian C. Gray, Wiley, 2003).

Thus, in an embodiment, a TVEMP disclosed herein comprises a Clostridial toxin enzymatic domain. In an aspect of this embodiment, a Clostridial toxin enzymatic domain comprises a naturally occurring Clostridial toxin enzymatic domain variant, such as, e.g., a Clostridial toxin enzymatic domain isoform or a Clostridial toxin enzymatic domain subtype. In another aspect of this embodiment, a Clostridial toxin enzymatic domain comprises a non-naturally occurring Clostridial toxin enzymatic domain variant, such as, e.g., a conservative Clostridial toxin enzymatic domain variant, a non-conservative Clostridial toxin enzymatic domain variant, an active Clostridial toxin enzymatic domain fragment, or any combination thereof.

In another embodiment, a hydrophic amino acid at one particular position in the polypeptide chain of the Clostridial toxin enzymatic domain can be substituted with another hydrophic amino acid. Examples of hydrophic amino acids include, e.g., C, F, I, L, M, V and W. In another aspect of this embodiment, an aliphatic amino acid at one particular position in the polypeptide chain of the Clostridial toxin enzymatic domain can be substituted with another aliphatic amino acid. Examples of aliphatic amino acids include, e.g., A, I, L, P, and V. In yet another aspect of this embodiment, an aromatic amino acid at one particular position in the polypeptide chain of the Clostridial toxin enzymatic domain can be substituted with another aromatic amino acid. Examples of aromatic amino acids include, e.g., F, H, W and Y. In still another aspect of this embodiment, a stacking amino acid at one particular position in the polypeptide chain of the Clostridial toxin enzymatic domain can be substituted with another stacking amino acid. Examples of stacking amino acids include, e.g., F, H, W and Y. In a further aspect of this embodiment, a polar amino acid at one particular position in the polypeptide chain of the Clostridial toxin enzymatic domain can be substituted with another polar amino acid. Examples of polar amino acids include, e.g., D, E, K, N, Q, and R. In a further aspect of this embodiment, a less polar or indifferent amino acid at one particular position in the polypeptide chain of the Clostridial toxin enzymatic domain can be substituted with another less polar or indifferent amino acid. Examples of less polar or indifferent amino acids include, e.g., A, H, G, P, S, T, and Y. In a yet further aspect of this embodiment, a positive charged amino acid at one particular position in the polypeptide chain of the Clostridial toxin enzymatic domain can be substituted with another positive charged amino acid. Examples of positive charged amino acids include, e.g., K, R, and H. In a still further aspect of this embodiment, a negative charged amino acid at one particular position in the polypeptide chain of the Clostridial toxin enzymatic domain can be substituted with another negative charged amino acid. Examples of negative charged amino acids include, e.g., D and E. In another aspect of this embodiment, a small amino acid at one particular position in the polypeptide chain of the Clostridial toxin enzymatic domain can be substituted with another small amino acid. Examples of small amino acids include, e.g., A, D, G, N, P, S, and T. In yet another aspect of this embodiment, a C-beta branching amino acid at one particular position in the polypeptide chain of the Clostridial toxin enzymatic domain can be substituted with another C-beta branching amino acid. Examples of C-beta branching amino acids include, e.g., I, T and V.

In another embodiment, a Clostridial toxin enzymatic domain comprises a BoNT/A enzymatic domain. In an aspect of this embodiment, a BoNT/A enzymatic domain comprises the enzymatic domains of SEQ ID NO: 1, SEQ ID NO: 2, SEQ ID NO: 3, SEQ ID NO: 4, or SEQ ID NO: 5. In other aspects of this embodiment, a BoNT/A enzymatic domain comprises amino acids 1/2-429 of SEQ ID NO: 1. In another aspect of this embodiment, a BoNT/A enzymatic domain comprises a naturally occurring BoNT/A enzymatic domain variant, such as, e.g., an enzymatic domain from a BoNT/A isoform or an enzymatic domain from a BoNT/A subtype. In another aspect of this embodiment, a BoNT/A enzymatic domain comprises a naturally occurring BoNT/A enzymatic domain variant of SEQ ID NO: 1, SEQ ID NO: 2, SEQ ID NO: 3, SEQ ID NO: 4, or SEQ ID NO: 5, such as, e.g., a BoNT/A isoform enzymatic domain or a BoNT/A subtype enzymatic domain. In another aspect of this embodiment, a BoNT/A enzymatic domain comprises amino acids 1/2-429 of a naturally occurring BoNT/A enzymatic domain variant of SEQ ID NO: 1, such as, e.g., a BoNT/A isoform enzymatic domain or a BoNT/A subtype enzymatic domain. In still another aspect of this embodiment, a BoNT/A enzymatic domain comprises a non-naturally occurring BoNT/A enzymatic domain variant, such as, e.g., a conservative BoNT/A enzymatic domain variant, a non-conservative BoNT/A enzymatic domain variant, an active BoNT/A enzymatic domain fragment, or any combination thereof. In still another aspect of this embodiment, a BoNT/A enzymatic domain comprises the enzymatic domain of a non-naturally occurring BoNT/A enzymatic domain variant of SEQ ID NO: 1, SEQ ID NO: 2, SEQ ID NO: 3, SEQ ID NO: 4, or SEQ ID NO: 5, such as, e.g., a conservative BoNT/A enzymatic domain variant, a non-conservative BoNT/A enzymatic domain variant, an active BoNT/A enzymatic domain fragment, or any combination thereof. In still another aspect of this embodiment, a BoNT/A enzymatic domain comprises amino acids 1/2-429 of a non-naturally occurring BoNT/A enzymatic domain variant of SEQ ID NO: 1, such as, e.g., a conservative BoNT/A enzymatic domain variant, a non-conservative BoNT/A enzymatic domain variant, an active BoNT/A enzymatic domain fragment, or any combination thereof.

In other aspects of this embodiment, a BoNT/A enzymatic domain comprises a polypeptide having an amino acid identity of, e.g., at least 70%, at least 75%, at least 80%, at least 85%, at least 90%, or at least 95% to the enzymatic domain of SEQ ID NO: 1, SEQ ID NO: 2, SEQ ID NO: 3, SEQ ID NO: 4, or SEQ ID NO: 5; or at most 70%, at most 75%, at most 80%, at most 85%, at most 90%, or at most 95% to the enzymatic domain of SEQ ID NO: 1, SEQ ID NO: 2, SEQ ID NO: 3, SEQ ID NO: 4, or SEQ ID NO: 5. In yet other aspects of this embodiment, a BoNT/A enzymatic domain comprises a polypeptide having an amino acid identity of, e.g., at least 70%, at least 75%, at least 80%, at least 85%, at least 90%, or at least 95% to amino acids 1/2-429 of SEQ ID NO: 1; or at most 70%, at most 75%, at most 80%, at most 85%, at most 90%, or at most 95% to amino acids 1/2-429 of SEQ ID NO: 1.

In other aspects of this embodiment, a BoNT/A enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 1, SEQ ID NO: 2, SEQ ID NO: 3, SEQ ID NO: 4, or SEQ ID NO: 5; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 1, SEQ ID NO: 2, SEQ ID NO: 3, SEQ ID NO: 4, or SEQ ID NO: 5. In yet other aspects of this embodiment, a BoNT/A enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1/2-429 of SEQ ID NO: 1; or at most 1, 2, 3, 4, 5, 6, 7, 8, 9, 10, 20, 30, 40, 50, or 100 non-contiguous amino acid deletions, additions, and/or substitutions relative to amino acids 1/2-429 of SEQ ID NO: 1. In still other aspects of this embodiment, a BoNT/A enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 1, SEQ ID NO: 2, SEQ ID NO: 3, SEQ ID NO: 4, or SEQ ID NO: 5; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 1, SEQ ID NO: 2, SEQ ID NO: 3, SEQ ID NO: 4, or SEQ ID NO: 5. In further other aspects of this embodiment, a BoNT/A enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1/2-429 of SEQ ID NO: 1; or at most 1, 2, 3, 4, 5, 6, 7, 8, 9, 10, 20, 30, 40, 50, or 100 contiguous amino acid deletions, additions, and/or substitutions relative to amino acids 1/2-429 of SEQ ID NO: 1.

In another embodiment, a Clostridial toxin enzymatic domain comprises a BoNT/B enzymatic domain. In an aspect of this embodiment, a BoNT/B enzymatic domain comprises the enzymatic domains of SEQ ID NO: 6, SEQ ID NO: 7, SEQ ID NO: 8, SEQ ID NO: 9, or SEQ ID NO: 10. In other aspects of this embodiment, a BoNT/B enzymatic domain comprises amino acids 1/2-436 of SEQ ID NO: 6. In another aspect of this embodiment, a BoNT/B enzymatic domain comprises a naturally occurring BoNT/B enzymatic domain variant, such as, e.g., an enzymatic domain from a BoNT/B isoform or an enzymatic domain from a BoNT/B subtype. In another aspect of this embodiment, a BoNT/B enzymatic domain comprises a naturally occurring BoNT/B enzymatic domain variant of SEQ ID NO: 6, SEQ ID NO: 7, SEQ ID NO: 8, SEQ ID NO: 9, or SEQ ID NO: 10, such as, e.g., a BoNT/B isoform enzymatic domain or a BoNT/B subtype enzymatic domain. In another aspect of this embodiment, a BoNT/B enzymatic domain comprises amino acids 1/2-436 of a naturally occurring BoNT/B enzymatic domain variant of SEQ ID NO: 6, such as, e.g., a BoNT/B isoform enzymatic domain or a BoNT/B subtype enzymatic domain. In still another aspect of this embodiment, a BoNT/B enzymatic domain comprises a non-naturally occurring BoNT/B enzymatic domain variant, such as, e.g., a conservative BoNT/B enzymatic domain variant, a non-conservative BoNT/B enzymatic domain variant, an active BoNT/B enzymatic domain fragment, or any combination thereof. In still another aspect of this embodiment, a BoNT/B enzymatic domain comprises the enzymatic domain of a non-naturally occurring BoNT/B enzymatic domain variant of SEQ ID NO: 6, SEQ ID NO: 7, SEQ ID NO: 8, SEQ ID NO: 9, or SEQ ID NO: 10, such as, e.g., a conservative BoNT/B enzymatic domain variant, a non-conservative BoNT/B enzymatic domain variant, an active BoNT/B enzymatic domain fragment, or any combination thereof. In still another aspect of this embodiment, a BoNT/B enzymatic domain comprises amino acids 1/2-436 of a non-naturally occurring BoNT/B enzymatic domain variant of SEQ ID NO: 6, such as, e.g., a conservative BoNT/B enzymatic domain variant, a non-conservative BoNT/B enzymatic domain variant, an active BoNT/B enzymatic domain fragment, or any combination thereof.

In other aspects of this embodiment, a BoNT/B enzymatic domain comprises a polypeptide having an amino acid identity of, e.g., at least 70%, at least 75%, at least 80%, at least 85%, at least 90%, or at least 95% to the enzymatic domain of SEQ ID NO: 6, SEQ ID NO: 7, SEQ ID NO: 8, SEQ ID NO: 9, or SEQ ID NO: 10; or at most 70%, at most 75%, at most 80%, at most 85%, at most 90%, or at most 95% to the enzymatic domain of SEQ ID NO: 6, SEQ ID NO: 7, SEQ ID NO: 8, SEQ ID NO: 9, or SEQ ID NO: 10. In yet other aspects of this embodiment, a BoNT/B enzymatic domain comprises a polypeptide having an amino acid identity of, e.g., at least 70%, at least 75%, at least 80%, at least 85%, at least 90%, or at least 95% to amino acids 1/2-436 of SEQ ID NO: 6; or at most 70%, at most 75%, at most 80%, at most 85%, at most 90%, or at most 95% to amino acids 1/2-436 of SEQ ID NO: 6.

In other aspects of this embodiment, a BoNT/B enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 6, SEQ ID NO: 7, SEQ ID NO: 8, SEQ ID NO: 9, or SEQ ID NO: 10; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 6, SEQ ID NO: 7, SEQ ID NO: 8, SEQ ID NO: 9, or SEQ ID NO: 10. In yet other aspects of this embodiment, a BoNT/B enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1/2-436 of SEQ ID NO: 6; or at most 1, 2, 3, 4, 5, 6, 7, 8, 9, 10, 20, 30, 40, 50, or 100 non-contiguous amino acid deletions, additions, and/or substitutions relative to amino acids 1/2-436 of SEQ ID NO: 6. In still other aspects of this embodiment, a BoNT/B enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 6, SEQ ID NO: 7, SEQ ID NO: 8, SEQ ID NO: 9, or SEQ ID NO: 10; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 6, SEQ ID NO: 7, SEQ ID NO: 8, SEQ ID NO: 9, or SEQ ID NO: 10. In further other aspects of this embodiment, a BoNT/B enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1/2-436 of SEQ ID NO: 6; or at most 1, 2, 3, 4, 5, 6, 7, 8, 9, 10, 20, 30, 40, 50, or 100 contiguous amino acid deletions, additions, and/or substitutions relative to amino acids 1/2-436 of SEQ ID NO: 6.

In another embodiment, a Clostridial toxin enzymatic domain comprises a BoNT/C1 enzymatic domain. In an aspect of this embodiment, a BoNT/C1 enzymatic domain comprises the enzymatic domains of SEQ ID NO: 11 or SEQ ID NO: 12. In other aspects of this embodiment, a BoNT/C1 enzymatic domain comprises amino acids 1/2-436 of SEQ ID NO: 11. In another aspect of this embodiment, a BoNT/C1 enzymatic domain comprises a naturally occurring BoNT/C1 enzymatic domain variant, such as, e.g., an enzymatic domain from a BoNT/C1 isoform or an enzymatic domain from a BoNT/C1 subtype. In another aspect of this embodiment, a BoNT/C1 enzymatic domain comprises a naturally occurring BoNT/C1 enzymatic domain variant of SEQ ID NO: 11 or SEQ ID NO: 12, such as, e.g., a BoNT/C1 isoform enzymatic domain or a BoNT/C1 subtype enzymatic domain. In another aspect of this embodiment, a BoNT/C1 enzymatic domain comprises amino acids 1/2-436 of a naturally occurring BoNT/C1 enzymatic domain variant of SEQ ID NO: 11, such as, e.g., a BoNT/C1 isoform enzymatic domain or a BoNT/C1 subtype enzymatic domain. In still another aspect of this embodiment, a BoNT/C1 enzymatic domain comprises a non-naturally occurring BoNT/C1 enzymatic domain variant, such as, e.g., a conservative BoNT/C1 enzymatic domain variant, a non-conservative BoNT/C1 enzymatic domain variant, an active BoNT/C1 enzymatic domain fragment, or any combination thereof. In still another aspect of this embodiment, a BoNT/C1 enzymatic domain comprises the enzymatic domain of a non-naturally occurring BoNT/C1 enzymatic domain variant of SEQ ID NO: 11 or SEQ ID NO: 12, such as, e.g., a conservative BoNT/C1 enzymatic domain variant, a non-conservative BoNT/C1 enzymatic domain variant, an active BoNT/C1 enzymatic domain fragment, or any combination thereof. In still another aspect of this embodiment, a BoNT/C1 enzymatic domain comprises amino acids 1/2-436 of a non-naturally occurring BoNT/C1 enzymatic domain variant of SEQ ID NO: 11, such as, e.g., a conservative BoNT/C1 enzymatic domain variant, a non-conservative BoNT/C1 enzymatic domain variant, an active BoNT/C1 enzymatic domain fragment, or any combination thereof.

In other aspects of this embodiment, a BoNT/C1 enzymatic domain comprises a polypeptide having an amino acid identity of, e.g., at least 70%, at least 75%, at least 80%, at least 85%, at least 90%, or at least 95% to the enzymatic domain of SEQ ID NO: 11 or SEQ ID NO: 12; or at most 70%, at most 75%, at most 80%, at most 85%, at most 90%, or at most 95% to the enzymatic domain of SEQ ID NO: 11 or SEQ ID NO: 12. In yet other aspects of this embodiment, a BoNT/C1 enzymatic domain comprises a polypeptide having an amino acid identity of, e.g., at least 70%, at least 75%, at least 80%, at least 85%, at least 90%, or at least 95% to amino acids 1/2-436 of SEQ ID NO: 11; or at most 70%, at most 75%, at most 80%, at most 85%, at most 90%, or at most 95% to amino acids 1/2-436 of SEQ ID NO: 11.

In other aspects of this embodiment, a BoNT/C1 enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 11 or SEQ ID NO: 12; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 11 or SEQ ID NO: 12. In yet other aspects of this embodiment, a BoNT/C1 enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1/2-436 of SEQ ID NO: 11; or at most 1, 2, 3, 4, 5, 6, 7, 8, 9, 10, 20, 30, 40, 50, or 100 non-contiguous amino acid deletions, additions, and/or substitutions relative to amino acids 1/2-436 of SEQ ID NO: 11. In still other aspects of this embodiment, a BoNT/C1 enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 11 or SEQ ID NO: 12; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 11 or SEQ ID NO: 12. In further other aspects of this embodiment, a BoNT/C1 enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1/2-436 of SEQ ID NO: 11; or at most 1, 2, 3, 4, 5, 6, 7, 8, 9, 10, 20, 30, 40, 50, or 100 contiguous amino acid deletions, additions, and/or substitutions relative to amino acids 1/2-436 of SEQ ID NO: 11.

In another embodiment, a Clostridial toxin enzymatic domain comprises a BoNT/D enzymatic domain. In an aspect of this embodiment, a BoNT/D enzymatic domain comprises the enzymatic domains of SEQ ID NO: 13 or SEQ ID NO: 14. In other aspects of this embodiment, a BoNT/D enzymatic domain comprises amino acids 1/2-436 of SEQ ID NO: 13. In another aspect of this embodiment, a BoNT/D enzymatic domain comprises a naturally occurring BoNT/D enzymatic domain variant, such as, e.g., an enzymatic domain from a BoNT/D isoform or an enzymatic domain from a BoNT/D subtype. In another aspect of this embodiment, a BoNT/D enzymatic domain comprises a naturally occurring BoNT/D enzymatic domain variant of SEQ ID NO: 13 or SEQ ID NO: 14, such as, e.g., a BoNT/D isoform enzymatic domain or a BoNT/D subtype enzymatic domain. In another aspect of this embodiment, a BoNT/D enzymatic domain comprises amino acids 1/2-436 of a naturally occurring BoNT/D enzymatic domain variant of SEQ ID NO: 13, such as, e.g., a BoNT/D isoform enzymatic domain or a BoNT/D subtype enzymatic domain. In still another aspect of this embodiment, a BoNT/D enzymatic domain comprises a non-naturally occurring BoNT/D enzymatic domain variant, such as, e.g., a conservative BoNT/D enzymatic domain variant, a non-conservative BoNT/D enzymatic domain variant, an active BoNT/D enzymatic domain fragment, or any combination thereof. In still another aspect of this embodiment, a BoNT/D enzymatic domain comprises the enzymatic domain of a non-naturally occurring BoNT/D enzymatic domain variant of SEQ ID NO: 13 or SEQ ID NO: 14, such as, e.g., a conservative BoNT/D enzymatic domain variant, a non-conservative BoNT/D enzymatic domain variant, an active BoNT/D enzymatic domain fragment, or any combination thereof. In still another aspect of this embodiment, a BoNT/D enzymatic domain comprises amino acids 1/2-436 of a non-naturally occurring BoNT/D enzymatic domain variant of SEQ ID NO: 13, such as, e.g., a conservative BoNT/D enzymatic domain variant, a non-conservative BoNT/D enzymatic domain variant, an active BoNT/D enzymatic domain fragment, or any combination thereof.

In other aspects of this embodiment, a BoNT/D enzymatic domain comprises a polypeptide having an amino acid identity of, e.g., at least 70%, at least 75%, at least 80%, at least 85%, at least 90%, or at least 95% to the enzymatic domain of SEQ ID NO: 13 or SEQ ID NO: 14; or at most 70%, at most 75%, at most 80%, at most 85%, at most 90%, or at most 95% to the enzymatic domain of SEQ ID NO: 13 or SEQ ID NO: 14. In yet other aspects of this embodiment, a BoNT/D enzymatic domain comprises a polypeptide having an amino acid identity of, e.g., at least 70%, at least 75%, at least 80%, at least 85%, at least 90%, or at least 95% to amino acids 1/2-436 of SEQ ID NO: 13; or at most 70%, at most 75%, at most 80%, at most 85%, at most 90%, or at most 95% to amino acids 1/2-436 of SEQ ID NO: 13.

In other aspects of this embodiment, a BoNT/D enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 13 or SEQ ID NO: 14; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 13 or SEQ ID NO: 14. In yet other aspects of this embodiment, a BoNT/D enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1/2-436 of SEQ ID NO: 13; or at most 1, 2, 3, 4, 5, 6, 7, 8, 9, 10, 20, 30, 40, 50, or 100 non-contiguous amino acid deletions, additions, and/or substitutions relative to amino acids 1/2-436 of SEQ ID NO: 13. In still other aspects of this embodiment, a BoNT/D enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 13 or SEQ ID NO: 14; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 13 or SEQ ID NO: 14. In further other aspects of this embodiment, a BoNT/D enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1/2-436 of SEQ ID NO: 13; or at most 1, 2, 3, 4, 5, 6, 7, 8, 9, 10, 20, 30, 40, 50, or 100 contiguous amino acid deletions, additions, and/or substitutions relative to amino acids 1/2-436 of SEQ ID NO: 13.

In another embodiment, a Clostridial toxin enzymatic domain comprises a BoNT/E enzymatic domain. In an aspect of this embodiment, a BoNT/E enzymatic domain comprises the enzymatic domains of SEQ ID NO: 15, SEQ ID NO: 16, or SEQ ID NO: 17. In other aspects of this embodiment, a BoNT/E enzymatic domain comprises amino acids 1/2-411 of SEQ ID NO: 15. In another aspect of this embodiment, a BoNT/E enzymatic domain comprises a naturally occurring BoNT/E enzymatic domain variant, such as, e.g., an enzymatic domain from a BoNT/E isoform or an enzymatic domain from a BoNT/E subtype. In another aspect of this embodiment, a BoNT/E enzymatic domain comprises a naturally occurring BoNT/E enzymatic domain variant of SEQ ID NO: 15, SEQ ID NO: 16, or SEQ ID NO: 17, such as, e.g., a BoNT/E isoform enzymatic domain or a BoNT/E subtype enzymatic domain. In another aspect of this embodiment, a BoNT/E enzymatic domain comprises amino acids 1/2-411 of a naturally occurring BoNT/E enzymatic domain variant of SEQ ID NO: 15, such as, e.g., a BoNT/E isoform enzymatic domain or a BoNT/E subtype enzymatic domain. In still another aspect of this embodiment, a BoNT/E enzymatic domain comprises a non-naturally occurring BoNT/E enzymatic domain variant, such as, e.g., a conservative BoNT/E enzymatic domain variant, a non-conservative BoNT/E enzymatic domain variant, an active BoNT/E enzymatic domain fragment, or any combination thereof. In still another aspect of this embodiment, a BoNT/E enzymatic domain comprises the enzymatic domain of a non-naturally occurring BoNT/E enzymatic domain variant of SEQ ID NO: 15, SEQ ID NO: 16, or SEQ ID NO: 17, such as, e.g., a conservative BoNT/E enzymatic domain variant, a non-conservative BoNT/E enzymatic domain variant, an active BoNT/E enzymatic domain fragment, or any combination thereof. In still another aspect of this embodiment, a BoNT/E enzymatic domain comprises amino acids 1/2-411 of a non-naturally occurring BoNT/E enzymatic domain variant of SEQ ID NO: 15, such as, e.g., a conservative BoNT/E enzymatic domain variant, a non-conservative BoNT/E enzymatic domain variant, an active BoNT/E enzymatic domain fragment, or any combination thereof.

In other aspects of this embodiment, a BoNT/E enzymatic domain comprises a polypeptide having an amino acid identity of, e.g., at least 70%, at least 75%, at least 80%, at least 85%, at least 90%, or at least 95% to the enzymatic domain of SEQ ID NO: 15, SEQ ID NO: 16, or SEQ ID NO: 17; or at most 70%, at most 75%, at most 80%, at most 85%, at most 90%, or at most 95% to the enzymatic domain of SEQ ID NO: 15, SEQ ID NO: 16, or SEQ ID NO: 17. In yet other aspects of this embodiment, a BoNT/E enzymatic domain comprises a polypeptide having an amino acid identity of, e.g., at least 70%, at least 75%, at least 80%, at least 85%, at least 90%, or at least 95% to amino acids 1/2-411 of SEQ ID NO: 15; or at most 70%, at most 75%, at most 80%, at most 85%, at most 90%, or at most 95% to amino acids 1/2-411 of SEQ ID NO: 15.

In other aspects of this embodiment, a BoNT/E enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 15, SEQ ID NO: 16, or SEQ ID NO: 17; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 15, SEQ ID NO: 16, or SEQ ID NO: 17. In yet other aspects of this embodiment, a BoNT/E enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1/2-411 of SEQ ID NO: 15; or at most 1, 2, 3, 4, 5, 6, 7, 8, 9, 10, 20, 30, 40, 50, or 100 non-contiguous amino acid deletions, additions, and/or substitutions relative to amino acids 1/2-411 of SEQ ID NO: 15. In still other aspects of this embodiment, a BoNT/E enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 15, SEQ ID NO: 16, or SEQ ID NO: 17; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 15, SEQ ID NO: 16, or SEQ ID NO: 17. In further other aspects of this embodiment, a BoNT/E enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1/2-411 of SEQ ID NO: 15; or at most 1, 2, 3, 4, 5, 6, 7, 8, 9, 10, 20, 30, 40, 50, or 100 contiguous amino acid deletions, additions, and/or substitutions relative to amino acids 1/2-411 of SEQ ID NO: 15.

In another embodiment, a Clostridial toxin enzymatic domain comprises a BoNT/F enzymatic domain. In an aspect of this embodiment, a BoNT/F enzymatic domain comprises the enzymatic domains of SEQ ID NO: 18, SEQ ID NO: 19, or SEQ ID NO: 20. In other aspects of this embodiment, a BoNT/F enzymatic domain comprises amino acids 1/2-428 of SEQ ID NO: 18. In another aspect of this embodiment, a BoNT/F enzymatic domain comprises a naturally occurring BoNT/F enzymatic domain variant, such as, e.g., an enzymatic domain from a BoNT/F isoform or an enzymatic domain from a BoNT/F subtype. In another aspect of this embodiment, a BoNT/F enzymatic domain comprises a naturally occurring BoNT/F enzymatic domain variant of SEQ ID NO: 18, SEQ ID NO: 19, or SEQ ID NO: 20, such as, e.g., a BoNT/F isoform enzymatic domain or a BoNT/F subtype enzymatic domain. In another aspect of this embodiment, a BoNT/F enzymatic domain comprises amino acids 1/2-428 of a naturally occurring BoNT/F enzymatic domain variant of SEQ ID NO: 18, such as, e.g., a BoNT/F isoform enzymatic domain or a BoNT/F subtype enzymatic domain. In still another aspect of this embodiment, a BoNT/F enzymatic domain comprises a non-naturally occurring BoNT/F enzymatic domain variant, such as, e.g., a conservative BoNT/F enzymatic domain variant, a non-conservative BoNT/F enzymatic domain variant, an active BoNT/F enzymatic domain fragment, or any combination thereof. In still another aspect of this embodiment, a BoNT/F enzymatic domain comprises the enzymatic domain of a non-naturally occurring BoNT/F enzymatic domain variant of SEQ ID NO: 18, SEQ ID NO: 19, or SEQ ID NO: 20, such as, e.g., a conservative BoNT/F enzymatic domain variant, a non-conservative BoNT/F enzymatic domain variant, an active BoNT/F enzymatic domain fragment, or any combination thereof. In still another aspect of this embodiment, a BoNT/F enzymatic domain comprises amino acids 1/2-428 of a non-naturally occurring BoNT/F enzymatic domain variant of SEQ ID NO: 18, such as, e.g., a conservative BoNT/F enzymatic domain variant, a non-conservative BoNT/F enzymatic domain variant, an active BoNT/F enzymatic domain fragment, or any combination thereof.

In other aspects of this embodiment, a BoNT/F enzymatic domain comprises a polypeptide having an amino acid identity of, e.g., at least 70%, at least 75%, at least 80%, at least 85%, at least 90%, or at least 95% to the enzymatic domain of SEQ ID NO: 18, SEQ ID NO: 19, or SEQ ID NO: 20; or at most 70%, at most 75%, at most 80%, at most 85%, at most 90%, or at most 95% to the enzymatic domain of SEQ ID NO: 18, SEQ ID NO: 19, or SEQ ID NO: 20. In yet other aspects of this embodiment, a BoNT/F enzymatic domain comprises a polypeptide having an amino acid identity of, e.g., at least 70%, at least 75%, at least 80%, at least 85%, at least 90%, or at least 95% to amino acids 1/2-428 of SEQ ID NO: 18; or at most 70%, at most 75%, at most 80%, at most 85%, at most 90%, or at most 95% to amino acids 1/2-428 of SEQ ID NO: 18.

In other aspects of this embodiment, a BoNT/F enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 18, SEQ ID NO: 19, or SEQ ID NO: 20; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 18, SEQ ID NO: 19, or SEQ ID NO: 20. In yet other aspects of this embodiment, a BoNT/F enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1/2-428 of SEQ ID NO: 18; or at most 1, 2, 3, 4, 5, 6, 7, 8, 9, 10, 20, 30, 40, 50, or 100 non-contiguous amino acid deletions, additions, and/or substitutions relative to amino acids 1/2-428 of SEQ ID NO: 18. In still other aspects of this embodiment, a BoNT/F enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 18, SEQ ID NO: 19, or SEQ ID NO: 20; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 18, SEQ ID NO: 19, or SEQ ID NO: 20. In further other aspects of this embodiment, a BoNT/F enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1/2-428 of SEQ ID NO: 18; or at most 1, 2, 3, 4, 5, 6, 7, 8, 9, 10, 20, 30, 40, 50, or 100 contiguous amino acid deletions, additions, and/or substitutions relative to amino acids 1/2-428 of SEQ ID NO: 18.

In another embodiment, a Clostridial toxin enzymatic domain comprises a BoNT/G enzymatic domain. In an aspect of this embodiment, a BoNT/G enzymatic domain comprises the enzymatic domains of SEQ ID NO: 21. In other aspects of this embodiment, a BoNT/G enzymatic domain comprises amino acids 1/2-4435 of SEQ ID NO: 21. In another aspect of this embodiment, a BoNT/G enzymatic domain comprises a naturally occurring BoNT/G enzymatic domain variant, such as, e.g., an enzymatic domain from a BoNT/G isoform or an enzymatic domain from a BoNT/G subtype. In another aspect of this embodiment, a BoNT/G enzymatic domain comprises a naturally occurring BoNT/G enzymatic domain variant of SEQ ID NO: 21, such as, e.g., a BoNT/G isoform enzymatic domain or a BoNT/G subtype enzymatic domain. In another aspect of this embodiment, a BoNT/G enzymatic domain comprises amino acids 1/2-4435 of a naturally occurring BoNT/G enzymatic domain variant of SEQ ID NO: 21, such as, e.g., a BoNT/G isoform enzymatic domain or a BoNT/G subtype enzymatic domain. In still another aspect of this embodiment, a BoNT/G enzymatic domain comprises a non-naturally occurring BoNT/G enzymatic domain variant, such as, e.g., a conservative BoNT/G enzymatic domain variant, a non-conservative BoNT/G enzymatic domain variant, an active BoNT/G enzymatic domain fragment, or any combination thereof. In still another aspect of this embodiment, a BoNT/G enzymatic domain comprises the enzymatic domain of a non-naturally occurring BoNT/G enzymatic domain variant of SEQ ID NO: 21, such as, e.g., a conservative BoNT/G enzymatic domain variant, a non-conservative BoNT/G enzymatic domain variant, an active BoNT/G enzymatic domain fragment, or any combination thereof. In still another aspect of this embodiment, a BoNT/G enzymatic domain comprises amino acids 1/2-4435 of a non-naturally occurring BoNT/G enzymatic domain variant of SEQ ID NO: 21, such as, e.g., a conservative BoNT/G enzymatic domain variant, a non-conservative BoNT/G enzymatic domain variant, an active BoNT/G enzymatic domain fragment, or any combination thereof.

In other aspects of this embodiment, a BoNT/G enzymatic domain comprises a polypeptide having an amino acid identity of, e.g., at least 70%, at least 75%, at least 80%, at least 85%, at least 90%, or at least 95% to the enzymatic domain of SEQ ID NO: 21; or at most 70%, at most 75%, at most 80%, at most 85%, at most 90%, or at most 95% to the enzymatic domain of SEQ ID NO: 21. In yet other aspects of this embodiment, a BoNT/G enzymatic domain comprises a polypeptide having an amino acid identity of, e.g., at least 70%, at least 75%, at least 80%, at least 85%, at least 90%, or at least 95% to amino acids 1/2-4435 of SEQ ID NO: 21; or at most 70%, at most 75%, at most 80%, at most 85%, at most 90%, or at most 95% to amino acids 1/2-4435 of SEQ ID NO: 21.

In other aspects of this embodiment, a BoNT/G enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 21; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 21. In yet other aspects of this embodiment, a BoNT/G enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1/2-4435 of SEQ ID NO: 21; or at most 1, 2, 3, 4, 5, 6, 7, 8, 9, 10, 20, 30, 40, 50, or 100 non-contiguous amino acid deletions, additions, and/or substitutions relative to amino acids 1/2-4435 of SEQ ID NO: 21. In still other aspects of this embodiment, a BoNT/G enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 21; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 21. In further other aspects of this embodiment, a BoNT/G enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1/2-4435 of SEQ ID NO: 21; or at most 1, 2, 3, 4, 5, 6, 7, 8, 9, 10, 20, 30, 40, 50, or 100 contiguous amino acid deletions, additions, and/or substitutions relative to amino acids 1/2-4435 of SEQ ID NO: 21.

In another embodiment, a Clostridial toxin enzymatic domain comprises a TeNT enzymatic domain. In an aspect of this embodiment, a TeNT enzymatic domain comprises the enzymatic domains of SEQ ID NO: 22. In other aspects of this embodiment, a TeNT enzymatic domain comprises amino acids 1/2-438 of SEQ ID NO: 22. In another aspect of this embodiment, a TeNT enzymatic domain comprises a naturally occurring TeNT enzymatic domain variant, such as, e.g., an enzymatic domain from a TeNT isoform or an enzymatic domain from a TeNT subtype. In another aspect of this embodiment, a TeNT enzymatic domain comprises a naturally occurring TeNT enzymatic domain variant of SEQ ID NO: 22, such as, e.g., a TeNT isoform enzymatic domain or a TeNT subtype enzymatic domain. In another aspect of this embodiment, a TeNT enzymatic domain comprises amino acids 1/2-438 of a naturally occurring TeNT enzymatic domain variant of SEQ ID NO: 22, such as, e.g., a TeNT isoform enzymatic domain or a TeNT subtype enzymatic domain. In still another aspect of this embodiment, a TeNT enzymatic domain comprises a non-naturally occurring TeNT enzymatic domain variant, such as, e.g., a conservative TeNT enzymatic domain variant, a non-conservative TeNT enzymatic domain variant, an active TeNT enzymatic domain fragment, or any combination thereof. In still another aspect of this embodiment, a TeNT enzymatic domain comprises the enzymatic domain of a non-naturally occurring TeNT enzymatic domain variant of SEQ ID NO: 22, such as, e.g., a conservative TeNT enzymatic domain variant, a non-conservative TeNT enzymatic domain variant, an active TeNT enzymatic domain fragment, or any combination thereof. In still another aspect of this embodiment, a TeNT enzymatic domain comprises amino acids 1/2-438 of a non-naturally occurring TeNT enzymatic domain variant of SEQ ID NO: 22, such as, e.g., a conservative TeNT enzymatic domain variant, a non-conservative TeNT enzymatic domain variant, an active TeNT enzymatic domain fragment, or any combination thereof.

In other aspects of this embodiment, a TeNT enzymatic domain comprises a polypeptide having an amino acid identity of, e.g., at least 70%, at least 75%, at least 80%, at least 85%, at least 90%, or at least 95% to the enzymatic domain of SEQ ID NO: 22; or at most 70%, at most 75%, at most 80%, at most 85%, at most 90%, or at most 95% to the enzymatic domain of SEQ ID NO: 22. In yet other aspects of this embodiment, a TeNT enzymatic domain comprises a polypeptide having an amino acid identity of, e.g., at least 70%, at least 75%, at least 80%, at least 85%, at least 90%, or at least 95% to amino acids 1/2-438 of SEQ ID NO: 22; or at most 70%, at most 75%, at most 80%, at most 85%, at most 90%, or at most 95% to amino acids 1/2-438 of SEQ ID NO: 22.

In other aspects of this embodiment, a TeNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 22; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 22. In yet other aspects of this embodiment, a TeNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1/2-438 of SEQ ID NO: 22; or at most 1, 2, 3, 4, 5, 6, 7, 8, 9, 10, 20, 30, 40, 50, or 100 non-contiguous amino acid deletions, additions, and/or substitutions relative to amino acids 1/2-438 of SEQ ID NO: 22. In still other aspects of this embodiment, a TeNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 22; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 22. In further other aspects of this embodiment, a TeNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1/2-438 of SEQ ID NO: 22; or at most 1, 2, 3, 4, 5, 6, 7, 8, 9, 10, 20, 30, 40, 50, or 100 contiguous amino acid deletions, additions, and/or substitutions relative to amino acids 1/2-438 of SEQ ID NO: 22.

In another embodiment, a Clostridial toxin enzymatic domain comprises a BaNT enzymatic domain. In an aspect of this embodiment, a BaNT enzymatic domain comprises the enzymatic domains of SEQ ID NO: 23. In other aspects of this embodiment, a BaNT enzymatic domain comprises amino acids 1/2-420 of SEQ ID NO: 23. In another aspect of this embodiment, a BaNT enzymatic domain comprises a naturally occurring BaNT enzymatic domain variant, such as, e.g., an enzymatic domain from a BaNT isoform or an enzymatic domain from a BaNT subtype. In another aspect of this embodiment, a BaNT enzymatic domain comprises a naturally occurring BaNT enzymatic domain variant of SEQ ID NO: 23, such as, e.g., a BaNT isoform enzymatic domain or a BaNT subtype enzymatic domain. In another aspect of this embodiment, a BaNT enzymatic domain comprises amino acids 1/2-420 of a naturally occurring BaNT enzymatic domain variant of SEQ ID NO: 23, such as, e.g., a BaNT isoform enzymatic domain or a BaNT subtype enzymatic domain. In still another aspect of this embodiment, a BaNT enzymatic domain comprises a non-naturally occurring BaNT enzymatic domain variant, such as, e.g., a conservative BaNT enzymatic domain variant, a non-conservative BaNT enzymatic domain variant, an active BaNT enzymatic domain fragment, or any combination thereof. In still another aspect of this embodiment, a BaNT enzymatic domain comprises the enzymatic domain of a non-naturally occurring BaNT enzymatic domain variant of SEQ ID NO: 23, such as, e.g., a conservative BaNT enzymatic domain variant, a non-conservative BaNT enzymatic domain variant, an active BaNT enzymatic domain fragment, or any combination thereof. In still another aspect of this embodiment, a BaNT enzymatic domain comprises amino acids 1/2-420 of a non-naturally occurring BaNT enzymatic domain variant of SEQ ID NO: 23, such as, e.g., a conservative BaNT enzymatic domain variant, a non-conservative BaNT enzymatic domain variant, an active BaNT enzymatic domain fragment, or any combination thereof.

In other aspects of this embodiment, a BaNT enzymatic domain comprises a polypeptide having an amino acid identity of, e.g., at least 70%, at least 75%, at least 80%, at least 85%, at least 90%, or at least 95% to the enzymatic domain of SEQ ID NO: 23; or at most 70%, at most 75%, at most 80%, at most 85%, at most 90%, or at most 95% to the enzymatic domain of SEQ ID NO: 23. In yet other aspects of this embodiment, a BaNT enzymatic domain comprises a polypeptide having an amino acid identity of, e.g., at least 70%, at least 75%, at least 80%, at least 85%, at least 90%, or at least 95% to amino acids 1/2-420 of SEQ ID NO: 23; or at most 70%, at most 75%, at most 80%, at most 85%, at most 90%, or at most 95% to amino acids 1/2-420 of SEQ ID NO: 23.

In other aspects of this embodiment, a BaNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 23; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 23. In yet other aspects of this embodiment, a BaNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1/2-420 of SEQ ID NO: 23; or at most 1, 2, 3, 4, 5, 6, 7, 8, 9, 10, 20, 30, 40, 50, or 100 non-contiguous amino acid deletions, additions, and/or substitutions relative to amino acids 1/2-420 of SEQ ID NO: 23. In still other aspects of this embodiment, a BaNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 23; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 23. In further other aspects of this embodiment, a BaNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1/2-420 of SEQ ID NO: 23; or at most 1, 2, 3, 4, 5, 6, 7, 8, 9, 10, 20, 30, 40, 50, or 100 contiguous amino acid deletions, additions, and/or substitutions relative to amino acids 1/2-420 of SEQ ID NO: 23.

In another embodiment, a Clostridial toxin enzymatic domain comprises a BuNT enzymatic domain. In an aspect of this embodiment, a BuNT enzymatic domain comprises the enzymatic domains of SEQ ID NO: 24 or SEQ ID NO: 25. In other aspects of this embodiment, a BuNT enzymatic domain comprises amino acids 1/2-411 of SEQ ID NO: 24. In another aspect of this embodiment, a BuNT enzymatic domain comprises a naturally occurring BuNT enzymatic domain variant, such as, e.g., an enzymatic domain from a BuNT isoform or an enzymatic domain from a BuNT subtype. In another aspect of this embodiment, a BuNT enzymatic domain comprises a naturally occurring BuNT enzymatic domain variant of SEQ ID NO: 24 or SEQ ID NO: 25, such as, e.g., a BuNT isoform enzymatic domain or a BuNT subtype enzymatic domain. In another aspect of this embodiment, a BuNT enzymatic domain comprises amino acids 1/2-411 of a naturally occurring BuNT enzymatic domain variant of SEQ ID NO: 24, such as, e.g., a BuNT isoform enzymatic domain or a BuNT subtype enzymatic domain. In still another aspect of this embodiment, a BuNT enzymatic domain comprises a non-naturally occurring BuNT enzymatic domain variant, such as, e.g., a conservative BuNT enzymatic domain variant, a non-conservative BuNT enzymatic domain variant, an active BuNT enzymatic domain fragment, or any combination thereof. In still another aspect of this embodiment, a BuNT enzymatic domain comprises the enzymatic domain of a non-naturally occurring BuNT enzymatic domain variant of SEQ ID NO: 24 or SEQ ID NO: 25, such as, e.g., a conservative BuNT enzymatic domain variant, a non-conservative BuNT enzymatic domain variant, an active BuNT enzymatic domain fragment, or any combination thereof. In still another aspect of this embodiment, a BuNT enzymatic domain comprises amino acids 1/2-411 of a non-naturally occurring BuNT enzymatic domain variant of SEQ ID NO: 24, such as, e.g., a conservative BuNT enzymatic domain variant, a non-conservative BuNT enzymatic domain variant, an active BuNT enzymatic domain fragment, or any combination thereof.

In other aspects of this embodiment, a BuNT enzymatic domain comprises a polypeptide having an amino acid identity of, e.g., at least 70%, at least 75%, at least 80%, at least 85%, at least 90%, or at least 95% to the enzymatic domain of SEQ ID NO: 24 or SEQ ID NO: 25; or at most 70%, at most 75%, at most 80%, at most 85%, at most 90%, or at most 95% to the enzymatic domain of SEQ ID NO: 24 or SEQ ID NO: 25. In yet other aspects of this embodiment, a BuNT enzymatic domain comprises a polypeptide having an amino acid identity of, e.g., at least 70%, at least 75%, at least 80%, at least 85%, at least 90%, or at least 95% to amino acids 1/2-411 of SEQ ID NO: 24 or SEQ ID NO: 25; or at most 70%, at most 75%, at most 80%, at most 85%, at most 90%, or at most 95% to amino acids 1/2-411 of SEQ ID NO: 24 or SEQ ID NO: 25.

In other aspects of this embodiment, a BuNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 24 or SEQ ID NO: 25; or at most 1, 2, 3, 4, 5, 6, 7, 8, 9, 10, 20, 30, 40, 50, or 100 non-contiguous amino acid deletions, additions, and/or substitutions relative to the enzymatic domain of SEQ ID NO: 24 OR SEQ ID NO: 25. In yet other aspects of this embodiment, a BuNT enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1/2-411 of SEQ ID NO: 24 or SEQ ID NO: 25; or at most 1, 2, 3, 4, 5, 6, 7, 8, 9, 10, 20, 30, 40, 50, or 100 non-contiguous amino acid deletions, additions, and/or substitutions relative to amino acids 1/2-411 of SEQ ID NO: 24 or SEQ ID NO: 25. In still other aspects of this embodiment, a BuNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 24 or SEQ ID NO: 25; or at most 1, 2, 3, 4, 5, 6, 7, 8, 9, 10, 20, 30, 40, 50, or 100 contiguous amino acid deletions, additions, and/or substitutions relative to the enzymatic domain of SEQ ID NO: 24 or SEQ ID NO: 25. In further other aspects of this embodiment, a BuNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1/2-411 of SEQ ID NO: 24 or SEQ ID NO: 25; or at most 1, 2, 3, 4, 5, 6, 7, 8, 9, 10, 20, 30, 40, 50, or 100 contiguous amino acid deletions, additions, and/or substitutions relative to amino acids 1/2-411 of SEQ ID NO: 24 or SEQ ID NO: 25.

The “translocation domain” comprises a portion of a Clostridial neurotoxin heavy chain having a translocation activity. By “translocation” is meant the ability to facilitate the transport of a polypeptide through a vesicular membrane, thereby exposing some or all of the polypeptide to the cytoplasm. In the various botulinum neurotoxins translocation is thought to involve an allosteric conformational change of the heavy chain caused by a decrease in pH within the endosome. This conformational change appears to involve and be mediated by the N terminal half of the heavy chain and to result in the formation of pores in the vesicular membrane; this change permits the movement of the proteolytic light chain from within the endosomal vesicle into the cytoplasm. See e.g., Lacy, et al., Nature Struct. Biol. 5:898-902 (October 1998).

The amino acid sequence of the translocation-mediating portion of the botulinum neurotoxin heavy chain is known to those of skill in the art; additionally, those amino acid residues within this portion that are known to be essential for conferring the translocation activity are also known. It would therefore be well within the ability of one of ordinary skill in the art, for example, to employ the naturally occurring N-terminal peptide half of the heavy chain of any of the various Clostridium tetanus or Clostridium botulinum neurotoxin subtypes as a translocation domain, or to design an analogous translocation domain by aligning the primary sequences of the N-terminal halves of the various heavy chains and selecting a consensus primary translocation sequence based on conserved amino acid, polarity, steric and hydrophobicity characteristics between the sequences.

Aspects of the present specification provide, in part, a TVEMP comprising a Clostridial toxin translocation domain. As used herein, the term “Clostridial toxin translocation domain” refers to any Clostridial toxin polypeptide that can execute the translocation step of the intoxication process that mediates Clostridial toxin light chain translocation. Thus, a Clostridial toxin translocation domain facilitates the movement of a Clostridial toxin light chain across a membrane and encompasses the movement of a Clostridial toxin light chain through the membrane an intracellular vesicle into the cytoplasm of a cell. Non-limiting examples of a Clostridial toxin translocation domain include, e.g., a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, and a BuNT translocation domain.

A Clostridial toxin translocation domain includes, without limitation, naturally occurring Clostridial toxin translocation domain variants, such as, e.g., Clostridial toxin translocation domain isoforms and Clostridial toxin translocation domain subtypes; non-naturally occurring Clostridial toxin translocation domain variants, such as, e.g., conservative Clostridial toxin translocation domain variants, non-conservative Clostridial toxin translocation domain variants, active Clostridial toxin translocation domain fragments thereof, or any combination thereof.

As used herein, the term “Clostridial toxin translocation domain variant,” whether naturally-occurring or non-naturally-occurring, refers to a Clostridial toxin translocation domain that has at least one amino acid change from the corresponding region of the disclosed reference sequences (Table 1) and can be described in percent identity to the corresponding region of that reference sequence. Unless expressly indicated, Clostridial toxin translocation domain variants useful to practice disclosed embodiments are variants that execute the translocation step of the intoxication process that mediates Clostridial toxin light chain translocation. As non-limiting examples, a BoNT/A translocation domain variant will have at least one amino acid difference, such as, e.g., an amino acid substitution, deletion or addition, as compared to amino acids 455-873 of SEQ ID NO: 1; a BoNT/B translocation domain variant will have at least one amino acid difference, such as, e.g., an amino acid substitution, deletion or addition, as compared to amino acids 447-860 of SEQ ID NO: 6; a BoNT/C1 translocation domain variant will have at least one amino acid difference, such as, e.g., an amino acid substitution, deletion or addition, as compared to amino acids 454-868 of SEQ ID NO: 11; a BoNT/D translocation domain variant will have at least one amino acid difference, such as, e.g., an amino acid substitution, deletion or addition, as compared to amino acids 451-864 of SEQ ID NO: 13; a BoNT/E translocation domain variant will have at least one amino acid difference, such as, e.g., an amino acid substitution, deletion or addition, as compared to amino acids 427-847 of SEQ ID NO: 15; a BoNT/F translocation domain variant will have at least one amino acid difference, such as, e.g., an amino acid substitution, deletion or addition, as compared to amino acids 446-865 of SEQ ID NO: 18; a BoNT/G translocation domain variant will have at least one amino acid difference, such as, e.g., an amino acid substitution, deletion or addition, as compared to amino acids 451-865 of SEQ ID NO: 21; a TeNT translocation domain variant will have at least one amino acid difference, such as, e.g., an amino acid substitution, deletion or addition, as compared to amino acids 468-881 of SEQ ID NO: 22; a BaNT translocation domain variant will have at least one amino acid difference, such as, e.g., an amino acid substitution, deletion or addition, as compared to amino acids 436-857 of SEQ ID NO: 23; and a BuNT translocation domain variant will have at least one amino acid difference, such as, e.g., an amino acid substitution, deletion or addition, as compared to amino acids 427-847 of SEQ ID NO: 24.

It is recognized by those of skill in the art that within each serotype of Clostridial toxin there can be naturally occurring Clostridial toxin translocation domain variants that differ somewhat in their amino acid sequence, and also in the nucleic acids encoding these proteins. For example, there are presently five BoNT/A subtypes, BoNT/A1, BoNT/A2, BoNT/A3, BoNT/A4, and BoNT/A5, with specific translocation domain subtypes showing about 85-87% amino acid identity when compared to the BoNT/A translocation domain subtype of SEQ ID NO: 1. As used herein, the term “naturally occurring Clostridial toxin translocation domain variant” refers to any Clostridial toxin translocation domain produced by a naturally-occurring process, including, without limitation, Clostridial toxin translocation domain isoforms produced from alternatively-spliced transcripts, Clostridial toxin translocation domain isoforms produced by spontaneous mutation and Clostridial toxin translocation domain subtypes. A naturally occurring Clostridial toxin translocation domain variant can function in substantially the same manner as the reference Clostridial toxin translocation domain on which the naturally occurring Clostridial toxin translocation domain variant is based, and can be substituted for the reference Clostridial toxin translocation domain in any aspect of the present specification.

A non-limiting examples of a naturally occurring Clostridial toxin translocation domain variant is a Clostridial toxin translocation domain isoform such as, e.g., a BoNT/A translocation domain isoform, a BoNT/B translocation domain isoform, a BoNT/C1 translocation domain isoform, a BoNT/D translocation domain isoform, a BoNT/E translocation domain isoform, a BoNT/F translocation domain isoform, a BoNT/G translocation domain isoform, a TeNT translocation domain isoform, a BaNT translocation domain isoform, and a BuNT translocation domain isoform. Another non-limiting examples of a naturally occurring Clostridial toxin translocation domain variant is a Clostridial toxin translocation domain subtype such as, e.g., a translocation domain from subtype BoNT/A1, BoNT/A2, BoNT/A3, BoNT/A4, and BoNT/A5; a translocation domain from subtype BoNT/B1, BoNT/B2, BoNT/B bivalent and BoNT/B nonproteolytic; a translocation domain from subtype BoNT/C1-1 and BoNT/C1-2; a translocation domain from subtype BoNT/E1, BoNT/E2 and BoNT/E3; a translocation domain from subtype BoNT/F1, BoNT/F2, BoNT/F3; and a translocation domain from subtype BuNT-1 and BuNT-2.

As used herein, the term “non-naturally occurring Clostridial toxin translocation domain variant” refers to any Clostridial toxin translocation domain produced with the aid of human manipulation, including, without limitation, Clostridial toxin translocation domains produced by genetic engineering using random mutagenesis or rational design and Clostridial toxin translocation domains produced by chemical synthesis. Non-limiting examples of non-naturally occurring Clostridial toxin translocation domain variants include, e.g., conservative Clostridial toxin translocation domain variants, non-conservative Clostridial toxin translocation domain variants, and active Clostridial toxin translocation domain fragments.

As used herein, the term “conservative Clostridial toxin translocation domain variant” refers to a Clostridial toxin translocation domain that has at least one amino acid substituted by another amino acid or an amino acid analog that has at least one property similar to that of the original amino acid from the reference Clostridial toxin translocation domain sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin translocation domain variant can function in substantially the same manner as the reference Clostridial toxin translocation domain on which the conservative Clostridial toxin translocation domain variant is based, and can be substituted for the reference Clostridial toxin translocation domain in any aspect of the present specification. Non-limiting examples of a conservative Clostridial toxin translocation domain variant include, e.g., conservative BoNT/A translocation domain variants, conservative BoNT/B translocation domain variants, conservative BoNT/C1 translocation domain variants, conservative BoNT/D translocation domain variants, conservative BoNT/E translocation domain variants, conservative BoNT/F translocation domain variants, conservative BoNT/G translocation domain variants, conservative TeNT translocation domain variants, conservative BaNT translocation domain variants, and conservative BuNT translocation domain variants.

As used herein, the term “non-conservative Clostridial toxin translocation domain variant” refers to a Clostridial toxin translocation domain in which 1) at least one amino acid is deleted from the reference Clostridial toxin translocation domain on which the non-conservative Clostridial toxin translocation domain variant is based; 2) at least one amino acid added to the reference Clostridial toxin translocation domain on which the non-conservative Clostridial toxin translocation domain is based; or 3) at least one amino acid is substituted by another amino acid or an amino acid analog that does not share any property similar to that of the original amino acid from the reference Clostridial toxin translocation domain sequence (Table 1). A non-conservative Clostridial toxin translocation domain variant can function in substantially the same manner as the reference Clostridial toxin translocation domain on which the non-conservative Clostridial toxin translocation domain variant is based, and can be substituted for the reference Clostridial toxin translocation domain in any aspect of the present specification. Non-limiting examples of a non-conservative Clostridial toxin translocation domain variant include, e.g., non-conservative BoNT/A translocation domain variants, non-conservative BoNT/B translocation domain variants, non-conservative BoNT/C1 translocation domain variants, non-conservative BoNT/D translocation domain variants, non-conservative BoNT/E translocation domain variants, non-conservative BoNT/F translocation domain variants, non-conservative BoNT/G translocation domain variants, and non-conservative TeNT translocation domain variants, non-conservative BaNT translocation domain variants, and non-conservative BuNT translocation domain variants.

As used herein, the term “active Clostridial toxin translocation domain fragment” refers to any of a variety of Clostridial toxin fragments comprising the translocation domain can be useful in aspects of the present specification with the proviso that these active fragments can facilitate the release of the LC from intracellular vesicles into the cytoplasm of the target cell and thus participate in executing the overall cellular mechanism whereby a Clostridial toxin proteolytically cleaves a substrate. The translocation domains from the heavy chains of Clostridial toxins are approximately 410-430 amino acids in length and comprise a translocation domain (Table 1). Research has shown that the entire length of a translocation domain from a Clostridial toxin heavy chain is not necessary for the translocating activity of the translocation domain. Thus, aspects of this embodiment include a Clostridial toxin translocation domain having a length of, e.g., at least 350, 375, 400, or 425 amino acids. Other aspects of this embodiment include a Clostridial toxin translocation domain having a length of, e.g., at most 350, 375, 400, or 425 amino acids.

Any of a variety of sequence alignment methods can be used to determine percent identity of naturally-occurring Clostridial toxin translocation domain variants and non-naturally-occurring Clostridial toxin translocation domain variants, including, without limitation, global methods, local methods and hybrid methods, such as, e.g., segment approach methods. Protocols to determine percent identity are routine procedures within the scope of one skilled in the art and from the teaching herein.

Thus, in an embodiment, a TVEMP disclosed herein comprises a Clostridial toxin translocation domain. In an aspect of this embodiment, a Clostridial toxin translocation domain comprises a naturally occurring Clostridial toxin translocation domain variant, such as, e.g., a Clostridial toxin translocation domain isoform or a Clostridial toxin translocation domain subtype. In another aspect of this embodiment, a Clostridial toxin translocation domain comprises a non-naturally occurring Clostridial toxin translocation domain variant, such as, e.g., a conservative Clostridial toxin translocation domain variant, a non-conservative Clostridial toxin translocation domain variant, an active Clostridial toxin translocation domain fragment, or any combination thereof.

In another embodiment, a hydrophic amino acid at one particular position in the polypeptide chain of the Clostridial toxin translocation domain can be substituted with another hydrophic amino acid. Examples of hydrophic amino acids include, e.g., C, F, I, L, M, V and W. In another aspect of this embodiment, an aliphatic amino acid at one particular position in the polypeptide chain of the Clostridial toxin translocation domain can be substituted with another aliphatic amino acid. Examples of aliphatic amino acids include, e.g., A, I, L, P, and V. In yet another aspect of this embodiment, an aromatic amino acid at one particular position in the polypeptide chain of the Clostridial toxin translocation domain can be substituted with another aromatic amino acid. Examples of aromatic amino acids include, e.g., F, H, W and Y. In still another aspect of this embodiment, a stacking amino acid at one particular position in the polypeptide chain of the Clostridial toxin translocation domain can be substituted with another stacking amino acid. Examples of stacking amino acids include, e.g., F, H, W and Y. In a further aspect of this embodiment, a polar amino acid at one particular position in the polypeptide chain of the Clostridial toxin translocation domain can be substituted with another polar amino acid. Examples of polar amino acids include, e.g., D, E, K, N, Q, and R. In a further aspect of this embodiment, a less polar or indifferent amino acid at one particular position in the polypeptide chain of the Clostridial toxin translocation domain can be substituted with another less polar or indifferent amino acid. Examples of less polar or indifferent amino acids include, e.g., A, H, G, P, S, T, and Y. In a yet further aspect of this embodiment, a positive charged amino acid at one particular position in the polypeptide chain of the Clostridial toxin translocation domain can be substituted with another positive charged amino acid. Examples of positive charged amino acids include, e.g., K, R, and H. In a still further aspect of this embodiment, a negative charged amino acid at one particular position in the polypeptide chain of the Clostridial toxin translocation domain can be substituted with another negative charged amino acid. Examples of negative charged amino acids include, e.g., D and E. In another aspect of this embodiment, a small amino acid at one particular position in the polypeptide chain of the Clostridial toxin translocation domain can be substituted with another small amino acid. Examples of small amino acids include, e.g., A, D, G, N, P, S, and T. In yet another aspect of this embodiment, a C-beta branching amino acid at one particular position in the polypeptide chain of the Clostridial toxin translocation domain can be substituted with another C-beta branching amino acid. Examples of C-beta branching amino acids include, e.g., I, T and V.

In another embodiment, a Clostridial toxin translocation domain comprises a BoNT/A translocation domain. In an aspect of this embodiment, a BoNT/A translocation domain comprises the translocation domains of SEQ ID NO: 1, SEQ ID NO: 2, SEQ ID NO: 3, SEQ ID NO: 4, or SEQ ID NO: 5. In other aspects of this embodiment, a BoNT/A translocation domain comprises amino acids 455-873 of SEQ ID NO: 1. In another aspect of this embodiment, a BoNT/A translocation domain comprises a naturally occurring BoNT/A translocation domain variant, such as, e.g., an translocation domain from a BoNT/A isoform or an translocation domain from a BoNT/A subtype. In another aspect of this embodiment, a BoNT/A translocation domain comprises a naturally occurring BoNT/A translocation domain variant of SEQ ID NO: 1, SEQ ID NO: 2, SEQ ID NO: 3, SEQ ID NO: 4, or SEQ ID NO: 5, such as, e.g., a BoNT/A isoform translocation domain or a BoNT/A subtype translocation domain. In another aspect of this embodiment, a BoNT/A translocation domain comprises amino acids 455-873 of a naturally occurring BoNT/A translocation domain variant of SEQ ID NO: 1, such as, e.g., a BoNT/A isoform translocation domain or a BoNT/A subtype translocation domain. In still another aspect of this embodiment, a BoNT/A translocation domain comprises a non-naturally occurring BoNT/A translocation domain variant, such as, e.g., a conservative BoNT/A translocation domain variant, a non-conservative BoNT/A translocation domain variant, an active BoNT/A translocation domain fragment, or any combination thereof. In still another aspect of this embodiment, a BoNT/A translocation domain comprises the translocation domain of a non-naturally occurring BoNT/A translocation domain variant of SEQ ID NO: 1, SEQ ID NO: 2, SEQ ID NO: 3, SEQ ID NO: 4, or SEQ ID NO: 5, such as, e.g., a conservative BoNT/A translocation domain variant, a non-conservative BoNT/A translocation domain variant, an active BoNT/A translocation domain fragment, or any combination thereof. In still another aspect of this embodiment, a BoNT/A translocation domain comprises amino acids 455-873 of a non-naturally occurring BoNT/A translocation domain variant of SEQ ID NO: 1, such as, e.g., a conservative BoNT/A translocation domain variant, a non-conservative BoNT/A translocation domain variant, an active BoNT/A translocation domain fragment, or any combination thereof.

In other aspects of this embodiment, a BoNT/A translocation domain comprises a polypeptide having an amino acid identity of, e.g., at least 70%, at least 75%, at least 80%, at least 85%, at least 90%, or at least 95% to the translocation domain of SEQ ID NO: 1, SEQ ID NO: 2, SEQ ID NO: 3, SEQ ID NO: 4, or SEQ ID NO: 5; or at most 70%, at most 75%, at most 80%, at most 85%, at most 90%, or at most 95% to the translocation domain of SEQ ID NO: 1, SEQ ID NO: 2, SEQ ID NO: 3, SEQ ID NO: 4, or SEQ ID NO: 5. In yet other aspects of this embodiment, a BoNT/A translocation domain comprises a polypeptide having an amino acid identity of, e.g., at least 70%, at least 75%, at least 80%, at least 85%, at least 90%, or at least 95% to amino acids 455-873 of SEQ ID NO: 1; or at most 70%, at most 75%, at most 80%, at most 85%, at most 90%, or at most 95% to amino acids 455-873 of SEQ ID NO: 1.

In other aspects of this embodiment, a BoNT/A translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 1, SEQ ID NO: 2, SEQ ID NO: 3, SEQ ID NO: 4, or SEQ ID NO: 5; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 1, SEQ ID NO: 2, SEQ ID NO: 3, SEQ ID NO: 4, or SEQ ID NO: 5. In yet other aspects of this embodiment, a BoNT/A translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 455-873 of SEQ ID NO: 1; or at most 1, 2, 3, 4, 5, 6, 7, 8, 9, 10, 20, 30, 40, 50, or 100 non-contiguous amino acid deletions, additions, and/or substitutions relative to amino acids 455-873 of SEQ ID NO: 1. In still other aspects of this embodiment, a BoNT/A translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 1, SEQ ID NO: 2, SEQ ID NO: 3, SEQ ID NO: 4, or SEQ ID NO: 5; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 1, SEQ ID NO: 2, SEQ ID NO: 3, SEQ ID NO: 4, or SEQ ID NO: 5. In further other aspects of this embodiment, a BoNT/A translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 455-873 of SEQ ID NO: 1; or at most 1, 2, 3, 4, 5, 6, 7, 8, 9, 10, 20, 30, 40, 50, or 100 contiguous amino acid deletions, additions, and/or substitutions relative to amino acids 455-873 of SEQ ID NO: 1.

In another embodiment, a Clostridial toxin translocation domain comprises a BoNT/B translocation domain. In an aspect of this embodiment, a BoNT/B translocation domain comprises the translocation domains of SEQ ID NO: 6, SEQ ID NO: 7, SEQ ID NO: 8, SEQ ID NO: 9, or SEQ ID NO: 10. In other aspects of this embodiment, a BoNT/B translocation domain comprises amino acids 447-860 of SEQ ID NO: 6. In another aspect of this embodiment, a BoNT/B translocation domain comprises a naturally occurring BoNT/B translocation domain variant, such as, e.g., an translocation domain from a BoNT/B isoform or an translocation domain from a BoNT/B subtype. In another aspect of this embodiment, a BoNT/B translocation domain comprises a naturally occurring BoNT/B translocation domain variant of SEQ ID NO: 6, SEQ ID NO: 7, SEQ ID NO: 8, SEQ ID NO: 9, or SEQ ID NO: 10, such as, e.g., a BoNT/B isoform translocation domain or a BoNT/B subtype translocation domain. In another aspect of this embodiment, a BoNT/B translocation domain comprises amino acids 447-860 of a naturally occurring BoNT/B translocation domain variant of SEQ ID NO: 6, such as, e.g., a BoNT/B isoform translocation domain or a BoNT/B subtype translocation domain. In still another aspect of this embodiment, a BoNT/B translocation domain comprises a non-naturally occurring BoNT/B translocation domain variant, such as, e.g., a conservative BoNT/B translocation domain variant, a non-conservative BoNT/B translocation domain variant, an active BoNT/B translocation domain fragment, or any combination thereof. In still another aspect of this embodiment, a BoNT/B translocation domain comprises the translocation domain of a non-naturally occurring BoNT/B translocation domain variant of SEQ ID NO: 6, SEQ ID NO: 7, SEQ ID NO: 8, SEQ ID NO: 9, or SEQ ID NO: 10, such as, e.g., a conservative BoNT/B translocation domain variant, a non-conservative BoNT/B translocation domain variant, an active BoNT/B translocation domain fragment, or any combination thereof. In still another aspect of this embodiment, a BoNT/B translocation domain comprises amino acids 447-860 of a non-naturally occurring BoNT/B translocation domain variant of SEQ ID NO: 6, such as, e.g., a conservative BoNT/B translocation domain variant, a non-conservative BoNT/B translocation domain variant, an active BoNT/B translocation domain fragment, or any combination thereof.

In other aspects of this embodiment, a BoNT/B translocation domain comprises a polypeptide having an amino acid identity of, e.g., at least 70%, at least 75%, at least 80%, at least 85%, at least 90%, or at least 95% to the translocation domain of SEQ ID NO: 6, SEQ ID NO: 7, SEQ ID NO: 8, SEQ ID NO: 9, or SEQ ID NO: 10; or at most 70%, at most 75%, at most 80%, at most 85%, at most 90%, or at most 95% to the translocation domain of SEQ ID NO: 6, SEQ ID NO: 7, SEQ ID NO: 8, SEQ ID NO: 9, or SEQ ID NO: 10. In yet other aspects of this embodiment, a BoNT/B translocation domain comprises a polypeptide having an amino acid identity of, e.g., at least 70%, at least 75%, at least 80%, at least 85%, at least 90%, or at least 95% to amino acids 447-860 of SEQ ID NO: 6; or at most 70%, at most 75%, at most 80%, at most 85%, at most 90%, or at most 95% to amino acids 447-860 of SEQ ID NO: 6.

In other aspects of this embodiment, a BoNT/B translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 6, SEQ ID NO: 7, SEQ ID NO: 8, SEQ ID NO: 9, or SEQ ID NO: 10; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 6, SEQ ID NO: 7, SEQ ID NO: 8, SEQ ID NO: 9, or SEQ ID NO: 10. In yet other aspects of this embodiment, a BoNT/B translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 447-860 of SEQ ID NO: 6; or at most 1, 2, 3, 4, 5, 6, 7, 8, 9, 10, 20, 30, 40, 50, or 100 non-contiguous amino acid deletions, additions, and/or substitutions relative to amino acids 447-860 of SEQ ID NO: 6. In still other aspects of this embodiment, a BoNT/B translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 6, SEQ ID NO: 7, SEQ ID NO: 8, SEQ ID NO: 9, or SEQ ID NO: 10; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 6, SEQ ID NO: 7, SEQ ID NO: 8, SEQ ID NO: 9, or SEQ ID NO: 10. In further other aspects of this embodiment, a BoNT/B translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 447-860 of SEQ ID NO: 6; or at most 1, 2, 3, 4, 5, 6, 7, 8, 9, 10, 20, 30, 40, 50, or 100 contiguous amino acid deletions, additions, and/or substitutions relative to amino acids 447-860 of SEQ ID NO: 6.

In another embodiment, a Clostridial toxin translocation domain comprises a BoNT/C1 translocation domain. In an aspect of this embodiment, a BoNT/C1 translocation domain comprises the translocation domains of SEQ ID NO: 11 or SEQ ID NO: 12. In other aspects of this embodiment, a BoNT/C1 translocation domain comprises amino acids 454-868 of SEQ ID NO: 11. In another aspect of this embodiment, a BoNT/C1 translocation domain comprises a naturally occurring BoNT/C1 translocation domain variant, such as, e.g., an translocation domain from a BoNT/C1 isoform or an translocation domain from a BoNT/C1 subtype. In another aspect of this embodiment, a BoNT/C1 translocation domain comprises a naturally occurring BoNT/C1 translocation domain variant of SEQ ID NO: 11 or SEQ ID NO: 12, such as, e.g., a BoNT/C1 isoform translocation domain or a BoNT/C1 subtype translocation domain. In another aspect of this embodiment, a BoNT/C1 translocation domain comprises amino acids 454-868 of a naturally occurring BoNT/C1 translocation domain variant of SEQ ID NO: 11, such as, e.g., a BoNT/C1 isoform translocation domain or a BoNT/C1 subtype translocation domain. In still another aspect of this embodiment, a BoNT/C1 translocation domain comprises a non-naturally occurring BoNT/C1 translocation domain variant, such as, e.g., a conservative BoNT/C1 translocation domain variant, a non-conservative BoNT/C1 translocation domain variant, an active BoNT/C1 translocation domain fragment, or any combination thereof. In still another aspect of this embodiment, a BoNT/C1 translocation domain comprises the translocation domain of a non-naturally occurring BoNT/C1 translocation domain variant of SEQ ID NO: 11 or SEQ ID NO: 12, such as, e.g., a conservative BoNT/C1 translocation domain variant, a non-conservative BoNT/C1 translocation domain variant, an active BoNT/C1 translocation domain fragment, or any combination thereof. In still another aspect of this embodiment, a BoNT/C1 translocation domain comprises amino acids 454-868 of a non-naturally occurring BoNT/C1 translocation domain variant of SEQ ID NO: 11, such as, e.g., a conservative BoNT/C1 translocation domain variant, a non-conservative BoNT/C1 translocation domain variant, an active BoNT/C1 translocation domain fragment, or any combination thereof.

In other aspects of this embodiment, a BoNT/C1 translocation domain comprises a polypeptide having an amino acid identity of, e.g., at least 70%, at least 75%, at least 80%, at least 85%, at least 90%, or at least 95% to the translocation domain of SEQ ID NO: 11 or SEQ ID NO: 12; or at most 70%, at most 75%, at most 80%, at most 85%, at most 90%, or at most 95% to the translocation domain of SEQ ID NO: 11 or SEQ ID NO: 12. In yet other aspects of this embodiment, a BoNT/C1 translocation domain comprises a polypeptide having an amino acid identity of, e.g., at least 70%, at least 75%, at least 80%, at least 85%, at least 90%, or at least 95% to amino acids 454-868 of SEQ ID NO: 11; or at most 70%, at most 75%, at most 80%, at most 85%, at most 90%, or at most 95% to amino acids 454-868 of SEQ ID NO: 11.

In other aspects of this embodiment, a BoNT/C1 translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 11 or SEQ ID NO: 12; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 11 or SEQ ID NO: 12. In yet other aspects of this embodiment, a BoNT/C1 translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 454-868 of SEQ ID NO: 11; or at most 1, 2, 3, 4, 5, 6, 7, 8, 9, 10, 20, 30, 40, 50, or 100 non-contiguous amino acid deletions, additions, and/or substitutions relative to amino acids 454-868 of SEQ ID NO: 11. In still other aspects of this embodiment, a BoNT/C1 translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 11 or SEQ ID NO: 12; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 11 or SEQ ID NO: 12. In further other aspects of this embodiment, a BoNT/C1 translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 454-868 of SEQ ID NO: 11; or at most 1, 2, 3, 4, 5, 6, 7, 8, 9, 10, 20, 30, 40, 50, or 100 contiguous amino acid deletions, additions, and/or substitutions relative to amino acids 454-868 of SEQ ID NO: 11.

In another embodiment, a Clostridial toxin translocation domain comprises a BoNT/D translocation domain. In an aspect of this embodiment, a BoNT/D translocation domain comprises the translocation domains of SEQ ID NO: 13 or SEQ ID NO: 14. In other aspects of this embodiment, a BoNT/D translocation domain comprises amino acids 451-864 of SEQ ID NO: 13. In another aspect of this embodiment, a BoNT/D translocation domain comprises a naturally occurring BoNT/D translocation domain variant, such as, e.g., an translocation domain from a BoNT/D isoform or an translocation domain from a BoNT/D subtype. In another aspect of this embodiment, a BoNT/D translocation domain comprises a naturally occurring BoNT/D translocation domain variant of SEQ ID NO: 13 or SEQ ID NO: 14, such as, e.g., a BoNT/D isoform translocation domain or a BoNT/D subtype translocation domain. In another aspect of this embodiment, a BoNT/D translocation domain comprises amino acids 451-864 of a naturally occurring BoNT/D translocation domain variant of SEQ ID NO: 13, such as, e.g., a BoNT/D isoform translocation domain or a BoNT/D subtype translocation domain. In still another aspect of this embodiment, a BoNT/D translocation domain comprises a non-naturally occurring BoNT/D translocation domain variant, such as, e.g., a conservative BoNT/D translocation domain variant, a non-conservative BoNT/D translocation domain variant, an active BoNT/D translocation domain fragment, or any combination thereof. In still another aspect of this embodiment, a BoNT/D translocation domain comprises the translocation domain of a non-naturally occurring BoNT/D translocation domain variant of SEQ ID NO: 13 or SEQ ID NO: 14, such as, e.g., a conservative BoNT/D translocation domain variant, a non-conservative BoNT/D translocation domain variant, an active BoNT/D translocation domain fragment, or any combination thereof. In still another aspect of this embodiment, a BoNT/D translocation domain comprises amino acids 451-864 of a non-naturally occurring BoNT/D translocation domain variant of SEQ ID NO: 13, such as, e.g., a conservative BoNT/D translocation domain variant, a non-conservative BoNT/D translocation domain variant, an active BoNT/D translocation domain fragment, or any combination thereof.

In other aspects of this embodiment, a BoNT/D translocation domain comprises a polypeptide having an amino acid identity of, e.g., at least 70%, at least 75%, at least 80%, at least 85%, at least 90%, or at least 95% to the translocation domain of SEQ ID NO: 13 or SEQ ID NO: 14; or at most 70%, at most 75%, at most 80%, at most 85%, at most 90%, or at most 95% to the translocation domain of SEQ ID NO: 13 or SEQ ID NO: 14. In yet other aspects of this embodiment, a BoNT/D translocation domain comprises a polypeptide having an amino acid identity of, e.g., at least 70%, at least 75%, at least 80%, at least 85%, at least 90%, or at least 95% to amino acids 451-864 of SEQ ID NO: 13; or at most 70%, at most 75%, at most 80%, at most 85%, at most 90%, or at most 95% to amino acids 451-864 of SEQ ID NO: 13.

In other aspects of this embodiment, a BoNT/D translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 13 or SEQ ID NO: 14; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 13 or SEQ ID NO: 14. In yet other aspects of this embodiment, a BoNT/D translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 451-864 of SEQ ID NO: 13; or at most 1, 2, 3, 4, 5, 6, 7, 8, 9, 10, 20, 30, 40, 50, or 100 non-contiguous amino acid deletions, additions, and/or substitutions relative to amino acids 451-864 of SEQ ID NO: 13. In still other aspects of this embodiment, a BoNT/D translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 13 or SEQ ID NO: 14; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 13 or SEQ ID NO: 14. In further other aspects of this embodiment, a BoNT/D translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 451-864 of SEQ ID NO: 13; or at most 1, 2, 3, 4, 5, 6, 7, 8, 9, 10, 20, 30, 40, 50, or 100 contiguous amino acid deletions, additions, and/or substitutions relative to amino acids 451-864 of SEQ ID NO: 13.

In another embodiment, a Clostridial toxin translocation domain comprises a BoNT/E translocation domain. In an aspect of this embodiment, a BoNT/E translocation domain comprises the translocation domains of SEQ ID NO: 15, SEQ ID NO: 16, or SEQ ID NO: 17. In other aspects of this embodiment, a BoNT/E translocation domain comprises amino acids 427-847 of SEQ ID NO: 15. In another aspect of this embodiment, a BoNT/E translocation domain comprises a naturally occurring BoNT/E translocation domain variant, such as, e.g., an translocation domain from a BoNT/E isoform or an translocation domain from a BoNT/E subtype. In another aspect of this embodiment, a BoNT/E translocation domain comprises a naturally occurring BoNT/E translocation domain variant of SEQ ID NO: 15, SEQ ID NO: 16, or SEQ ID NO: 17, such as, e.g., a BoNT/E isoform translocation domain or a BoNT/E subtype translocation domain. In another aspect of this embodiment, a BoNT/E translocation domain comprises amino acids 427-847 of a naturally occurring BoNT/E translocation domain variant of SEQ ID NO: 15, such as, e.g., a BoNT/E isoform translocation domain or a BoNT/E subtype translocation domain. In still another aspect of this embodiment, a BoNT/E translocation domain comprises a non-naturally occurring BoNT/E translocation domain variant, such as, e.g., a conservative BoNT/E translocation domain variant, a non-conservative BoNT/E translocation domain variant, an active BoNT/E translocation domain fragment, or any combination thereof. In still another aspect of this embodiment, a BoNT/E translocation domain comprises the translocation domain of a non-naturally occurring BoNT/E translocation domain variant of SEQ ID NO: 15, SEQ ID NO: 16, or SEQ ID NO: 17, such as, e.g., a conservative BoNT/E translocation domain variant, a non-conservative BoNT/E translocation domain variant, an active BoNT/E translocation domain fragment, or any combination thereof. In still another aspect of this embodiment, a BoNT/E translocation domain comprises amino acids 427-847 of a non-naturally occurring BoNT/E translocation domain variant of SEQ ID NO: 15, such as, e.g., a conservative BoNT/E translocation domain variant, a non-conservative BoNT/E translocation domain variant, an active BoNT/E translocation domain fragment, or any combination thereof.

In other aspects of this embodiment, a BoNT/E translocation domain comprises a polypeptide having an amino acid identity of, e.g., at least 70%, at least 75%, at least 80%, at least 85%, at least 90%, or at least 95% to the translocation domain of SEQ ID NO: 15, SEQ ID NO: 16, or SEQ ID NO: 17; or at most 70%, at most 75%, at most 80%, at most 85%, at most 90%, or at most 95% to the translocation domain of SEQ ID NO: 15, SEQ ID NO: 16, or SEQ ID NO: 17. In yet other aspects of this embodiment, a BoNT/E translocation domain comprises a polypeptide having an amino acid identity of, e.g., at least 70%, at least 75%, at least 80%, at least 85%, at least 90%, or at least 95% to amino acids 427-847 of SEQ ID NO: 15; or at most 70%, at most 75%, at most 80%, at most 85%, at most 90%, or at most 95% to amino acids 427-847 of SEQ ID NO: 15.

In other aspects of this embodiment, a BoNT/E translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 15, SEQ ID NO: 16, or SEQ ID NO: 17; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 15, SEQ ID NO: 16, or SEQ ID NO: 17. In yet other aspects of this embodiment, a BoNT/E translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 427-847 of SEQ ID NO: 15; or at most 1, 2, 3, 4, 5, 6, 7, 8, 9, 10, 20, 30, 40, 50, or 100 non-contiguous amino acid deletions, additions, and/or substitutions relative to amino acids 427-847 of SEQ ID NO: 15. In still other aspects of this embodiment, a BoNT/E translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 15, SEQ ID NO: 16, or SEQ ID NO: 17; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 15, SEQ ID NO: 16, or SEQ ID NO: 17. In further other aspects of this embodiment, a BoNT/E translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 427-847 of SEQ ID NO: 15; or at most 1, 2, 3, 4, 5, 6, 7, 8, 9, 10, 20, 30, 40, 50, or 100 contiguous amino acid deletions, additions, and/or substitutions relative to amino acids 427-847 of SEQ ID NO: 15.

In another embodiment, a Clostridial toxin translocation domain comprises a BoNT/F translocation domain. In an aspect of this embodiment, a BoNT/F translocation domain comprises the translocation domains of SEQ ID NO: 18, SEQ ID NO: 19, or SEQ ID NO: 20. In other aspects of this embodiment, a BoNT/F translocation domain comprises amino acids 446-865 of SEQ ID NO: 18. In another aspect of this embodiment, a BoNT/F translocation domain comprises a naturally occurring BoNT/F translocation domain variant, such as, e.g., an translocation domain from a BoNT/F isoform or an translocation domain from a BoNT/F subtype. In another aspect of this embodiment, a BoNT/F translocation domain comprises a naturally occurring BoNT/F translocation domain variant of SEQ ID NO: 18, SEQ ID NO: 19, or SEQ ID NO: 20, such as, e.g., a BoNT/F isoform translocation domain or a BoNT/F subtype translocation domain. In another aspect of this embodiment, a BoNT/F translocation domain comprises amino acids 446-865 of a naturally occurring BoNT/F translocation domain variant of SEQ ID NO: 18, such as, e.g., a BoNT/F isoform translocation domain or a BoNT/F subtype translocation domain. In still another aspect of this embodiment, a BoNT/F translocation domain comprises a non-naturally occurring BoNT/F translocation domain variant, such as, e.g., a conservative BoNT/F translocation domain variant, a non-conservative BoNT/F translocation domain variant, an active BoNT/F translocation domain fragment, or any combination thereof. In still another aspect of this embodiment, a BoNT/F translocation domain comprises the translocation domain of a non-naturally occurring BoNT/F translocation domain variant of SEQ ID NO: 18, SEQ ID NO: 19, or SEQ ID NO: 20, such as, e.g., a conservative BoNT/F translocation domain variant, a non-conservative BoNT/F translocation domain variant, an active BoNT/F translocation domain fragment, or any combination thereof. In still another aspect of this embodiment, a BoNT/F translocation domain comprises amino acids 446-865 of a non-naturally occurring BoNT/F translocation domain variant of SEQ ID NO: 18, such as, e.g., a conservative BoNT/F translocation domain variant, a non-conservative BoNT/F translocation domain variant, an active BoNT/F translocation domain fragment, or any combination thereof.

In other aspects of this embodiment, a BoNT/F translocation domain comprises a polypeptide having an amino acid identity of, e.g., at least 70%, at least 75%, at least 80%, at least 85%, at least 90%, or at least 95% to the translocation domain of SEQ ID NO: 18, SEQ ID NO: 19, or SEQ ID NO: 20; or at most 70%, at most 75%, at most 80%, at most 85%, at most 90%, or at most 95% to the translocation domain of SEQ ID NO: 18, SEQ ID NO: 19, or SEQ ID NO: 20. In yet other aspects of this embodiment, a BoNT/F translocation domain comprises a polypeptide having an amino acid identity of, e.g., at least 70%, at least 75%, at least 80%, at least 85%, at least 90%, or at least 95% to amino acids 446-865 of SEQ ID NO: 18; or at most 70%, at most 75%, at most 80%, at most 85%, at most 90%, or at most 95% to amino acids 446-865 of SEQ ID NO: 18.

In other aspects of this embodiment, a BoNT/F translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 18, SEQ ID NO: 19, or SEQ ID NO: 20; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 18, SEQ ID NO: 19, or SEQ ID NO: 20. In yet other aspects of this embodiment, a BoNT/F translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 446-865 of SEQ ID NO: 18; or at most 1, 2, 3, 4, 5, 6, 7, 8, 9, 10, 20, 30, 40, 50, or 100 non-contiguous amino acid deletions, additions, and/or substitutions relative to amino acids 446-865 of SEQ ID NO: 18. In still other aspects of this embodiment, a BoNT/F translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 18, SEQ ID NO: 19, or SEQ ID NO: 20; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 18, SEQ ID NO: 19, or SEQ ID NO: 20. In further other aspects of this embodiment, a BoNT/F translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 446-865 of SEQ ID NO: 18; or at most 1, 2, 3, 4, 5, 6, 7, 8, 9, 10, 20, 30, 40, 50, or 100 contiguous amino acid deletions, additions, and/or substitutions relative to amino acids 446-865 of SEQ ID NO: 18.

In another embodiment, a Clostridial toxin translocation domain comprises a BoNT/G translocation domain. In an aspect of this embodiment, a BoNT/G translocation domain comprises the translocation domains of SEQ ID NO: 21. In other aspects of this embodiment, a BoNT/G translocation domain comprises amino acids 451-865 of SEQ ID NO: 21. In another aspect of this embodiment, a BoNT/G translocation domain comprises a naturally occurring BoNT/G translocation domain variant, such as, e.g., an translocation domain from a BoNT/G isoform or an translocation domain from a BoNT/G subtype. In another aspect of this embodiment, a BoNT/G translocation domain comprises a naturally occurring BoNT/G translocation domain variant of SEQ ID NO: 21, such as, e.g., a BoNT/G isoform translocation domain or a BoNT/G subtype translocation domain. In another aspect of this embodiment, a BoNT/G translocation domain comprises amino acids 451-865 of a naturally occurring BoNT/G translocation domain variant of SEQ ID NO: 21, such as, e.g., a BoNT/G isoform translocation domain or a BoNT/G subtype translocation domain. In still another aspect of this embodiment, a BoNT/G translocation domain comprises a non-naturally occurring BoNT/G translocation domain variant, such as, e.g., a conservative BoNT/G translocation domain variant, a non-conservative BoNT/G translocation domain variant, an active BoNT/G translocation domain fragment, or any combination thereof. In still another aspect of this embodiment, a BoNT/G translocation domain comprises the translocation domain of a non-naturally occurring BoNT/G translocation domain variant of SEQ ID NO: 21, such as, e.g., a conservative BoNT/G translocation domain variant, a non-conservative BoNT/G translocation domain variant, an active BoNT/G translocation domain fragment, or any combination thereof. In still another aspect of this embodiment, a BoNT/G translocation domain comprises amino acids 451-865 of a non-naturally occurring BoNT/G translocation domain variant of SEQ ID NO: 21, such as, e.g., a conservative BoNT/G translocation domain variant, a non-conservative BoNT/G translocation domain variant, an active BoNT/G translocation domain fragment, or any combination thereof.

In other aspects of this embodiment, a BoNT/G translocation domain comprises a polypeptide having an amino acid identity of, e.g., at least 70%, at least 75%, at least 80%, at least 85%, at least 90%, or at least 95% to the translocation domain of SEQ ID NO: 21; or at most 70%, at most 75%, at most 80%, at most 85%, at most 90%, or at most 95% to the translocation domain of SEQ ID NO: 21. In yet other aspects of this embodiment, a BoNT/G translocation domain comprises a polypeptide having an amino acid identity of, e.g., at least 70%, at least 75%, at least 80%, at least 85%, at least 90%, or at least 95% to amino acids 451-865 of SEQ ID NO: 21; or at most 70%, at most 75%, at most 80%, at most 85%, at most 90%, or at most 95% to amino acids 451-865 of SEQ ID NO: 21.

In other aspects of this embodiment, a BoNT/G translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 21; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 21. In yet other aspects of this embodiment, a BoNT/G translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 451-865 of SEQ ID NO: 21; or at most 1, 2, 3, 4, 5, 6, 7, 8, 9, 10, 20, 30, 40, 50, or 100 non-contiguous amino acid deletions, additions, and/or substitutions relative to amino acids 451-865 of SEQ ID NO: 21. In still other aspects of this embodiment, a BoNT/G translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 21; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 21. In further other aspects of this embodiment, a BoNT/G translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 451-865 of SEQ ID NO: 21; or at most 1, 2, 3, 4, 5, 6, 7, 8, 9, 10, 20, 30, 40, 50, or 100 contiguous amino acid deletions, additions, and/or substitutions relative to amino acids 451-865 of SEQ ID NO: 21.

In another embodiment, a Clostridial toxin translocation domain comprises a TeNT translocation domain. In an aspect of this embodiment, a TeNT translocation domain comprises the translocation domains of SEQ ID NO: 22. In other aspects of this embodiment, a TeNT translocation domain comprises amino acids 468-881 of SEQ ID NO: 22. In another aspect of this embodiment, a TeNT translocation domain comprises a naturally occurring TeNT translocation domain variant, such as, e.g., an translocation domain from a TeNT isoform or an translocation domain from a TeNT subtype. In another aspect of this embodiment, a TeNT translocation domain comprises a naturally occurring TeNT translocation domain variant of SEQ ID NO: 22, such as, e.g., a TeNT isoform translocation domain or a TeNT subtype translocation domain. In another aspect of this embodiment, a TeNT translocation domain comprises amino acids 468-881 of a naturally occurring TeNT translocation domain variant of SEQ ID NO: 22, such as, e.g., a TeNT isoform translocation domain or a TeNT subtype translocation domain. In still another aspect of this embodiment, a TeNT translocation domain comprises a non-naturally occurring TeNT translocation domain variant, such as, e.g., a conservative TeNT translocation domain variant, a non-conservative TeNT translocation domain variant, an active TeNT translocation domain fragment, or any combination thereof. In still another aspect of this embodiment, a TeNT translocation domain comprises the translocation domain of a non-naturally occurring TeNT translocation domain variant of SEQ ID NO: 22, such as, e.g., a conservative TeNT translocation domain variant, a non-conservative TeNT translocation domain variant, an active TeNT translocation domain fragment, or any combination thereof. In still another aspect of this embodiment, a TeNT translocation domain comprises amino acids 468-881 of a non-naturally occurring TeNT translocation domain variant of SEQ ID NO: 22, such as, e.g., a conservative TeNT translocation domain variant, a non-conservative TeNT translocation domain variant, an active TeNT translocation domain fragment, or any combination thereof.

In other aspects of this embodiment, a TeNT translocation domain comprises a polypeptide having an amino acid identity of, e.g., at least 70%, at least 75%, at least 80%, at least 85%, at least 90%, or at least 95% to the translocation domain of SEQ ID NO: 22; or at most 70%, at most 75%, at most 80%, at most 85%, at most 90%, or at most 95% to the translocation domain of SEQ ID NO: 22. In yet other aspects of this embodiment, a TeNT translocation domain comprises a polypeptide having an amino acid identity of, e.g., at least 70%, at least 75%, at least 80%, at least 85%, at least 90%, or at least 95% to amino acids 468-881 of SEQ ID NO: 22; or at most 70%, at most 75%, at most 80%, at most 85%, at most 90%, or at most 95% to amino acids 468-881 of SEQ ID NO: 22.

In other aspects of this embodiment, a TeNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 22; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 22. In yet other aspects of this embodiment, a TeNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 468-881 of SEQ ID NO: 22; or at most 1, 2, 3, 4, 5, 6, 7, 8, 9, 10, 20, 30, 40, 50, or 100 non-contiguous amino acid deletions, additions, and/or substitutions relative to amino acids 468-881 of SEQ ID NO: 22. In still other aspects of this embodiment, a TeNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 22; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 22. In further other aspects of this embodiment, a TeNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 468-881 of SEQ ID NO: 22; or at most 1, 2, 3, 4, 5, 6, 7, 8, 9, 10, 20, 30, 40, 50, or 100 contiguous amino acid deletions, additions, and/or substitutions relative to amino acids 468-881 of SEQ ID NO: 22.

In another embodiment, a Clostridial toxin translocation domain comprises a BaNT translocation domain. In an aspect of this embodiment, a BaNT translocation domain comprises the translocation domains of SEQ ID NO: 23. In other aspects of this embodiment, a BaNT translocation domain comprises amino acids 436-857 of SEQ ID NO: 23. In another aspect of this embodiment, a BaNT translocation domain comprises a naturally occurring BaNT translocation domain variant, such as, e.g., an translocation domain from a BaNT isoform or an translocation domain from a BaNT subtype. In another aspect of this embodiment, a BaNT translocation domain comprises a naturally occurring BaNT translocation domain variant of SEQ ID NO: 23, such as, e.g., a BaNT isoform translocation domain or a BaNT subtype translocation domain. In another aspect of this embodiment, a BaNT translocation domain comprises amino acids 436-857 of a naturally occurring BaNT translocation domain variant of SEQ ID NO: 23, such as, e.g., a BaNT isoform translocation domain or a BaNT subtype translocation domain. In still another aspect of this embodiment, a BaNT translocation domain comprises a non-naturally occurring BaNT translocation domain variant, such as, e.g., a conservative BaNT translocation domain variant, a non-conservative BaNT translocation domain variant, an active BaNT translocation domain fragment, or any combination thereof. In still another aspect of this embodiment, a BaNT translocation domain comprises the translocation domain of a non-naturally occurring BaNT translocation domain variant of SEQ ID NO: 23, such as, e.g., a conservative BaNT translocation domain variant, a non-conservative BaNT translocation domain variant, an active BaNT translocation domain fragment, or any combination thereof. In still another aspect of this embodiment, a BaNT translocation domain comprises amino acids 436-857 of a non-naturally occurring BaNT translocation domain variant of SEQ ID NO: 23, such as, e.g., a conservative BaNT translocation domain variant, a non-conservative BaNT translocation domain variant, an active BaNT translocation domain fragment, or any combination thereof.

In other aspects of this embodiment, a BaNT translocation domain comprises a polypeptide having an amino acid identity of, e.g., at least 70%, at least 75%, at least 80%, at least 85%, at least 90%, or at least 95% to the translocation domain of SEQ ID NO: 23; or at most 70%, at most 75%, at most 80%, at most 85%, at most 90%, or at most 95% to the translocation domain of SEQ ID NO: 23. In yet other aspects of this embodiment, a BaNT translocation domain comprises a polypeptide having an amino acid identity of, e.g., at least 70%, at least 75%, at least 80%, at least 85%, at least 90%, or at least 95% to amino acids 436-857 of SEQ ID NO: 23; or at most 70%, at most 75%, at most 80%, at most 85%, at most 90%, or at most 95% to amino acids 436-857 of SEQ ID NO: 23.

In other aspects of this embodiment, a BaNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 23; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 23. In yet other aspects of this embodiment, a BaNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 436-857 of SEQ ID NO: 23; or at most 1, 2, 3, 4, 5, 6, 7, 8, 9, 10, 20, 30, 40, 50, or 100 non-contiguous amino acid deletions, additions, and/or substitutions relative to amino acids 436-857 of SEQ ID NO: 23. In still other aspects of this embodiment, a BaNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 23; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 23. In further other aspects of this embodiment, a BaNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 436-857 of SEQ ID NO: 23; or at most 1, 2, 3, 4, 5, 6, 7, 8, 9, 10, 20, 30, 40, 50, or 100 contiguous amino acid deletions, additions, and/or substitutions relative to amino acids 436-857 of SEQ ID NO: 23.

In another embodiment, a Clostridial toxin translocation domain comprises a BuNT translocation domain. In an aspect of this embodiment, a BuNT translocation domain comprises the translocation domains of SEQ ID NO: 24 or SEQ ID NO: 25. In other aspects of this embodiment, a BuNT translocation domain comprises amino acids 427-847 of SEQ ID NO: 24. In another aspect of this embodiment, a BuNT translocation domain comprises a naturally occurring BuNT translocation domain variant, such as, e.g., a translocation domain from a BuNT isoform or an translocation domain from a BuNT subtype. In another aspect of this embodiment, a BuNT translocation domain comprises a naturally occurring BuNT translocation domain variant of SEQ ID NO: 24 or SEQ ID NO: 25, such as, e.g., a BuNT isoform translocation domain or a BuNT subtype translocation domain. In another aspect of this embodiment, a BuNT translocation domain comprises amino acids 427-847 of a naturally occurring BuNT translocation domain variant of SEQ ID NO: 24, such as, e.g., a BuNT isoform translocation domain or a BuNT subtype translocation domain. In still another aspect of this embodiment, a BuNT translocation domain comprises a non-naturally occurring BuNT translocation domain variant, such as, e.g., a conservative BuNT translocation domain variant, a non-conservative BuNT translocation domain variant, an active BuNT translocation domain fragment, or any combination thereof. In still another aspect of this embodiment, a BuNT translocation domain comprises the translocation domain of a non-naturally occurring BuNT translocation domain variant of SEQ ID NO: 24 or SEQ ID NO: 25, such as, e.g., a conservative BuNT translocation domain variant, a non-conservative BuNT translocation domain variant, an active BuNT translocation domain fragment, or any combination thereof. In still another aspect of this embodiment, a BuNT translocation domain comprises amino acids 427-847 of a non-naturally occurring BuNT translocation domain variant of SEQ ID NO: 24, such as, e.g., a conservative BuNT translocation domain variant, a non-conservative BuNT translocation domain variant, an active BuNT translocation domain fragment, or any combination thereof.

In other aspects of this embodiment, a BuNT translocation domain comprises a polypeptide having an amino acid identity of, e.g., at least 70%, at least 75%, at least 80%, at least 85%, at least 90%, or at least 95% to the translocation domain of SEQ ID NO: 24 or SEQ ID NO: 25; or at most 70%, at most 75%, at most 80%, at most 85%, at most 90%, or at most 95% to the translocation domain of SEQ ID NO: 24 or SEQ ID NO: 25. In yet other aspects of this embodiment, a BuNT translocation domain comprises a polypeptide having an amino acid identity of, e.g., at least 70%, at least 75%, at least 80%, at least 85%, at least 90%, or at least 95% to amino acids 427-847 of SEQ ID NO: 24 or SEQ ID NO: 25; or at most 70%, at most 75%, at most 80%, at most 85%, at most 90%, or at most 95% to amino acids 427-847 of SEQ ID NO: 24 or SEQ ID NO: 25.

In other aspects of this embodiment, a BuNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 24 or SEQ ID NO: 25; or at most 1, 2, 3, 4, 5, 6, 7, 8, 9, 10, 20, 30, 40, 50, or 100 non-contiguous amino acid deletions, additions, and/or substitutions relative to the translocation domain of SEQ ID NO: 24 OR SEQ ID NO: 25. In yet other aspects of this embodiment, a BuNT translocation domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 427-847 of SEQ ID NO: 24 or SEQ ID NO: 25; or at most 1, 2, 3, 4, 5, 6, 7, 8, 9, 10, 20, 30, 40, 50, or 100 non-contiguous amino acid deletions, additions, and/or substitutions relative to amino acids 427-847 of SEQ ID NO: 24 or SEQ ID NO: 25. In still other aspects of this embodiment, a BuNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to the translocation domain of SEQ ID NO: 24 or SEQ ID NO: 25; or at most 1, 2, 3, 4, 5, 6, 7, 8, 9, 10, 20, 30, 40, 50, or 100 contiguous amino acid deletions, additions, and/or substitutions relative to the translocation domain of SEQ ID NO: 24 or SEQ ID NO: 25. In further other aspects of this embodiment, a BuNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 427-847 of SEQ ID NO: 24 or SEQ ID NO: 25; or at most 1, 2, 3, 4, 5, 6, 7, 8, 9, 10, 20, 30, 40, 50, or 100 contiguous amino acid deletions, additions, and/or substitutions relative to amino acids 427-847 of SEQ ID NO: 24 or SEQ ID NO: 25.

Aspects of the present specification provide, in part, a TVEMP comprising a targeting domain. As used herein, the term “targeting domain” is synonymous with “binding domain”, “ligand”, or “targeting moiety” and refers to an amino acid sequence region able to preferentially bind to a cell surface marker, like a receptor, characteristic of the target cell under physiological conditions. The cell surface marker may comprise a polypeptide, a polysaccharide, a lipid, a glycoprotein, a lipoprotein, or may have structural characteristics of more than one of these. As used herein, the term “preferentially interacts” refers to a molecule capable of binding to its target cell surface marker under physiological conditions, or in vitro conditions substantially approximating physiological conditions, to a statistically significantly greater degree relative to other, non-target cell surface marker. With reference to a targeting domain disclosed herein, there is a discriminatory binding of the targeting domain to its cognate receptor relative to other receptors.

In an embodiment, a binding domain that selectively binds a target receptor has a dissociation equilibrium constant (K_{D}) that is greater for the target receptor relative to a non-target receptor by, e.g., at least one-fold, at least two-fold, at least three-fold, at least four fold, at least five-fold, at least 10 fold, at least 50 fold, at least 100 fold, at least 1000, at least 10,000, or at least 100,000 fold.

An example of a targeting domain disclosed herein is a Prostate Specific Membrane Antigen (PSMA) binding peptide. A PSMA binding peptide is a peptide that can selectively bind to PSMA. PSMA is a type II transmembrane metallo-peptidase comprising a short cytoplasmic N-terminal domain, a transmembrane domain, and a large extracellular C-terminal domain with several potential N-glycosylation sites. This cell surface carboxypeptidase shares overall sequence similarity to glutamate carboxypeptidase II and has folate hydrolase and N-acetylated α-linked acidic dipeptidase (NAALDase) activity. Additionally, the extracellular C-terminal domain of PSMA shares sequence similarity to the transferrin receptor I and transferrin receptor II. This glycoprotein is internalized via a clathrin-dependent endocytic mechanism which is mediated by five N-terminal amino acids present in its cytoplasmic tail.

PSMA shows a remarkably cancer and hyperproliferation-restricted expression pattern. This protein is highly expressed in cells comprising benign prostatic hyperplasia (BPH) and prostate cancer, like a prostatic epithelial cancer, a prostatic intraepithelial neoplasia (PIN), and a prostatic adenocarcinoma, as well as in cells comprising the neovasculature of many nonprostatic solid tumors. Such neovasculature-associated with cancers include, without limitation, a gastric cancer, such as, e.g., a gastric adenocarcinoma or a gastric carcinoma, a colorectal cancer, such as, e.g., a colorectal adenocarcinoma or a colorectal carcinoma, and metastatic cancer originating from these sites. However, PSMA is not expressed by normal endothelial cells or other normal tissues. PSMA, therefore, represents an attractive candidate receptor for selectively targeted therapies for prostate and/or other solid tumors. In addition, because of its unique expression pattern limited to tumor-associated endothelial cells, PSMA may also be an interesting molecule for neovascular or angiogenesis targeting. As such, a TVEMP comprising a PSMA targeting domain would be effective in treating BPH, prostate cancer, gastric cancer, colorectal cancer and any other neovascular-associated tumors because the enriched source of PSMA would allow for preferential targeting of these disease-state cells relative to the surrounding normal cells.

Thus, in an embodiment, a targeting domain comprises a PSMA targeting domain. In aspects of this embodiment, a PSMA targeting domain comprises SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112.

In other aspects of this embodiment, a PSMA targeting domain comprises a polypeptide having an amino acid identity of, e.g., at least 70%, at least 75%, at least 80%, at least 85%, at least 90% or at least 95% to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112; or at most 70%, at most 75%, at most 80%, at most 85%, at most 90% or at most 95% to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112.

In yet other aspects of this embodiment, a PSMA targeting domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112; or at most 1, 2, 3, 4, 5, 6, 7, 8, 9, 10, 15 or 20 non-contiguous amino acid deletions, additions, and/or substitutions relative to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112.

In still other aspects of this embodiment, a PSMA targeting domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112; or at most 1, 2, 3, 4, 5, 6, 7, 8, 9, 10, 15 or 20 contiguous amino acid deletions, additions, and/or substitutions relative to SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112.

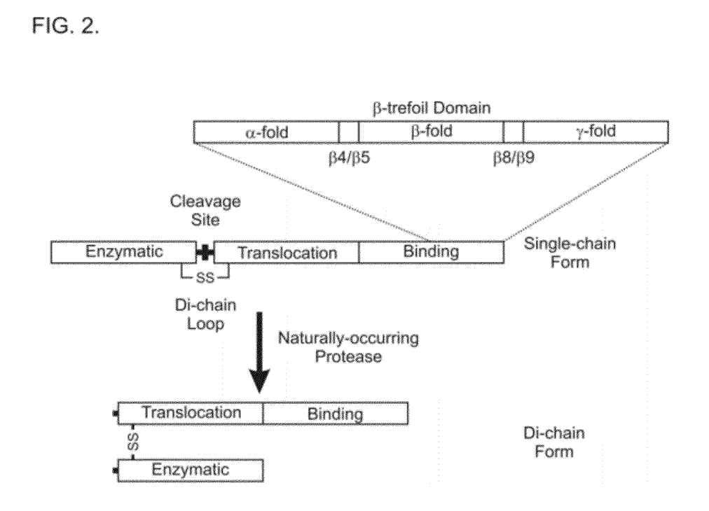

Clostridial toxins are each translated as a single-chain polypeptide of approximately 150 kDa that is subsequently cleaved by proteolytic scission within a disulfide loop by a naturally-occurring protease. This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains (FIG. 2). To facilitate recombinant production of a TVEMP, an exogenous protease cleavage site can be used to convert the single-chain polypeptide form of a TVEMP disclosed herein into the di-chain form. See, e.g., Steward, L. E. et al., Activatable Clostridial Toxins, U.S. Pat. No. 7,740,868; Dolly, J. O. et al., Activatable Clostridial Neurotoxins, U.S. Pat. No. 7,709,228; Steward, L. E. et al., Activatable Clostridial Toxins, U.S. Patent Publication 2009/0069238; Foster, K. A. et al., Fusion Proteins, US Patent Publication 2009/0035822; and Foster, K. A. et al., Non-Cytotoxic Protein Conjugates, US Patent Publication 2008/0187960; Steward, L. E. et al., Activatable Clostridial Toxins, U.S. Patent Publication 2008/0161226, each of which is incorporated by reference in its entirety.

In is envisioned that any and all protease cleavage sites can be used to convert the single-chain polypeptide form of a Clostridial toxin into the di-chain form, including, without limitation, endogenous di-chain loop protease cleavage sites and exogenous protease cleavage sites. Thus, in an aspect of the invention, a TVEMP comprises, in part, an endogenous protease cleavage site within a di-chain loop region. In another aspect of the invention, a TVEMP comprises, in part, an exogenous protease cleavage site within a di-chain loop region. As used herein, the term “di-chain loop region” means the amino acid sequence of a Clostridial toxin containing a protease cleavage site used to convert the single-chain form of a Clostridial toxin into the di-chain form. Non-limiting examples of a Clostridial toxin di-chain loop region, include, a di-chain loop region of BoNT/A comprising amino acids 430-454 of SEQ ID NO: 1; a di-chain loop region of BoNT/B comprising amino acids 437-446 of SEQ ID NO: 2; a di-chain loop region of BoNT/C1 comprising amino acids 437-453 of SEQ ID NO: 3; a di-chain loop region of BoNT/D comprising amino acids 437-450 of SEQ ID NO: 4; a di-chain loop region of BoNT/E comprising amino acids 412-426 of SEQ ID NO: 5; a di-chain loop region of BoNT/F comprising amino acids 429-445 of SEQ ID NO: 6; a di-chain loop region of BoNT/G comprising amino acids 436-450 of SEQ ID NO: 7; and a di-chain loop region of TeNT comprising amino acids 439-467 of SEQ ID NO: 8 (Table 4).

TABLE 4
Di-chain Loop Region of Clostridial Toxins

| | SEQ | Di-chain Loop Region Containing the |
|---|---|---|
| Toxin | ID NO: | Naturally-occurring Protease Cleavage Site |
| BoNT/A | 26 | CVRGIITSKTKSLDKGYNK*----ALNDLC |
| BoNT/B | 27 | CKSVK*-------------------APGIC |
| BoNT/C1 | 28 | CHKAIDGRSLYNK*------------TLDC |
| BoNT/D | 29 | CLRLTKNSR*---------------DDSTC |
| BoNT/E | 30 | CKNIVSVKGIR*--------------KSIC |
| BoNT/F | 31 | CKSVIPRKGTK*------------APPRLC |
| BoNT/G | 32 | CKPVMYKNTGK*--------------SEQC |
| TeNT | 33 | CKKIIPPTNIRENLYNRTA*SLTDLGGELC |
| BaNT | 34 | CKS-IVSKKGTK*-------------NSLC |
| BuNT | 35 | CKN-IVSVKGIR*-------------KSIC |

The amino acid sequence displayed are as follows: BoNT/A, residues 430-454 of SEQ ID NO: 1; BoNT/B, residues 437-446 of SEQ ID NO: 2; BoNT/C1, residues 437-453 of SEQ ID NO: 3; BoNT/D, residues 437-450 of SEQ ID NO: 4; BoNT/E, residues 412-426 of SEQ ID NO: 5; BoNT/F, residues 429- 445 of SEQ ID NO: 6; BoNT/G, residues 436-450 of SEQ ID NO: 7; TeNT, residues 439-467 of SEQ ID NO: 8; BaNT, residues 421-435 of SEQ ID NO: 9; and BuNT, residues 412-426 of SEQ ID NO: 10. An asterisks (*) indicates the peptide bond that is cleaved by a Clostridial toxin protease.

As used herein, the term “endogenous di-chain loop protease cleavage site” is synonymous with a “naturally occurring di-chain loop protease cleavage site” and means a naturally occurring protease cleavage site found within the di-chain loop region of a naturally occurring Clostridial toxin and includes, without limitation, naturally occurring Clostridial toxin di-chain loop protease cleavage site variants, such as, e.g., Clostridial toxin di-chain loop protease cleavage site isoforms and Clostridial toxin di-chain loop protease cleavage site subtypes. Non-limiting examples of an endogenous protease cleavage site, include, e.g., a BoNT/A di-chain loop protease cleavage site, a BoNT/B di-chain loop protease cleavage site, a BoNT/C1 di-chain loop protease cleavage site, a BoNT/D di-chain loop protease cleavage site, a BoNT/E di-chain loop protease cleavage site, a BoNT/F di-chain loop protease cleavage site, a BoNT/G di-chain loop protease cleavage site and a TeNT di-chain loop protease cleavage site.

As mentioned above, Clostridial toxins are translated as a single-chain polypeptide of approximately 150 kDa that is subsequently cleaved by proteolytic scission within a disulfide loop by a naturally-occurring protease. This posttranslational processing yields a di-chain molecule comprising an approximately 50 kDa light chain (LC) and an approximately 100 kDa heavy chain (HC) held together by a single disulphide bond and noncovalent interactions. While the identity of the protease is currently unknown, the di-chain loop protease cleavage site for many Clostridial toxins has been determined. In BoNTs, cleavage at K448-A449 converts the single polypeptide form of BoNT/A into the di-chain form; cleavage at K441-A442 converts the single polypeptide form of BoNT/B into the di-chain form; cleavage at K449-T450 converts the single polypeptide form of BoNT/C1 into the di-chain form; cleavage at R445-D446 converts the single polypeptide form of BoNT/D into the di-chain form; cleavage at R422-K423 converts the single polypeptide form of BoNT/E into the di-chain form; cleavage at K439-A440 converts the single polypeptide form of BoNT/F into the di-chain form; and cleavage at K446-S447 converts the single polypeptide form of BoNT/G into the di-chain form. Proteolytic cleavage of the single polypeptide form of TeNT at A457-S458 results in the di-chain form. Proteolytic cleavage of the single polypeptide form of BaNT at K431-N432 results in the di-chain form. Proteolytic cleavage of the single polypeptide form of BuNT at R422-K423 results in the di-chain form. Such a di-chain loop protease cleavage site is operably-linked in-frame to a TVEMP as a fusion protein. However, it should also be noted that additional cleavage sites within the di-chain loop also appear to be cleaved resulting in the generation of a small peptide fragment being lost. As a non-limiting example, BoNT/A single-chain polypeptide cleave ultimately results in the loss of a ten amino acid fragment within the di-chain loop.

Thus, in an embodiment, a protease cleavage site comprising an endogenous Clostridial toxin di-chain loop protease cleavage site is used to convert the single-chain toxin into the di-chain form. In aspects of this embodiment, conversion into the di-chain form by proteolytic cleavage occurs from a site comprising, e.g., a BoNT/A di-chain loop protease cleavage site, a BoNT/B di-chain loop protease cleavage site, a BoNT/C1 di-chain loop protease cleavage site, a BoNT/D di-chain loop protease cleavage site, a BoNT/E di-chain loop protease cleavage site, a BoNT/F di-chain loop protease cleavage site, a BoNT/G di-chain loop protease cleavage site, a TeNT di-chain loop protease cleavage site, a BaNT di-chain loop protease cleavage site, or a BuNT di-chain loop protease cleavage site.

In other aspects of this embodiment, conversion into the di-chain form by proteolytic cleavage occurs from a site comprising, e.g., a di-chain loop region of BoNT/A comprising amino acids 430-454 of SEQ ID NO: 1; a di-chain loop region of BoNT/B comprising amino acids 437-446 of SEQ ID NO: 2; a di-chain loop region of BoNT/C1 comprising amino acids 437-453 of SEQ ID NO: 3; a di-chain loop region of BoNT/D comprising amino acids 437-450 of SEQ ID NO: 4; a di-chain loop region of BoNT/E comprising amino acids 412-426 of SEQ ID NO: 5; a di-chain loop region of BoNT/F comprising amino acids 429-445 of SEQ ID NO: 6; a di-chain loop region of BoNT/G comprising amino acids 436-450 of SEQ ID NO: 7; or a di-chain loop region of TeNT comprising amino acids 439-467 of SEQ ID NO: 8. a di-chain loop region of BaNT comprising amino acids 421-435 of SEQ ID NO: 9; or a di-chain loop region of BuNT comprising amino acids 412-426 of SEQ ID NO: 10.

It is also envisioned that an exogenous protease cleavage site can be used to convert the single-chain polypeptide form of a TVEMP disclosed herein into the di-chain form. As used herein, the term “exogenous protease cleavage site” is synonymous with a “non-naturally occurring protease cleavage site” or “non-native protease cleavage site” and means a protease cleavage site that is not normally present in a di-chain loop region from a naturally occurring Clostridial toxin, with the proviso that the exogenous protease cleavage site is not a human protease cleavage site or a protease cleavage site that is susceptible to a protease being expressed in the host cell that is expressing a construct encoding an activatable polypeptide disclosed herein. It is envisioned that any and all exogenous protease cleavage sites can be used to convert the single-chain polypeptide form of a Clostridial toxin into the di-chain form are useful to practice aspects of the present invention. Non-limiting examples of exogenous protease cleavage sites include, e.g., a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus (TEV) protease cleavage site, a Tobacco Vein Mottling Virus (TVMV) cleavage site, a subtilisin cleavage site, a hydroxylamine cleavage site, or a Caspase 3 cleavage site.

It is envisioned that an exogenous protease cleavage site of any and all lengths can be useful in aspects of the present invention with the proviso that the exogenous protease cleavage site is capable of being cleaved by its respective protease. Thus, in aspects of this embodiment, an exogenous protease cleavage site can have a length of, e.g., at least 6, 7, 8, 9, 10, 15, 20, 25, 30, 40, 50, or at least 60 amino acids; or at most 6, 7, 8, 9, 10, 15, 20, 25, 30, 40, 50, or at least 60 amino acids.

In an embodiment, an exogenous protease cleavage site is located within the di-chain loop of a TVEMP. In aspects of this embodiment, a TVEMP comprises an exogenous protease cleavage site comprises, e.g., a plant papain cleavage site, an insect papain cleavage site, a crustacian papain cleavage site, a non-human enterokinase protease cleavage site, a Tobacco Etch Virus protease cleavage site, a Tobacco Vein Mottling Virus protease cleavage site, a human rhinovirus 3C protease cleavage site, a human enterovirus 3C protease cleavage site, a subtilisin cleavage site, a hydroxylamine cleavage site, a SUMO/ULP-1 protease cleavage site, and a non-human Caspase 3 cleavage site. In other aspects of this embodiment, an exogenous protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In an aspect of this embodiment, an exogenous protease cleavage site can comprise, e.g., a non-human enterokinase cleavage site is located within the di-chain loop of a TVEMP. In other aspects of the embodiment, an exogenous protease cleavage site can comprise, e.g., a bovine enterokinase protease cleavage site located within the di-chain loop of a TVEMP. In other aspects of the embodiment, an exogenous protease cleavage site can comprise, e.g., a bovine enterokinase protease cleavage site located within the di-chain loop of a TVEMP comprises SEQ ID NO: 36. In still other aspects of this embodiment, a bovine enterokinase protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In another aspect of this embodiment, an exogenous protease cleavage site can comprise, e.g., a Tobacco Etch Virus protease cleavage site is located within the di-chain loop of a TVEMP. In other aspects of the embodiment, an exogenous protease cleavage site can comprise, e.g., a Tobacco Etch Virus protease cleavage site located within the di-chain loop of a TVEMP comprises the consensus sequence E-P5-P4-Y-P2-Q*-G (SEQ ID NO: 377) or E-P5-P4-Y-P2-Q*-S (SEQ ID NO: 38), where P2, P4 and P5 can be any amino acid. In other aspects of the embodiment, an exogenous protease cleavage site can comprise, e.g., a Tobacco Etch Virus protease cleavage site located within the di-chain loop of a TVEMP comprises SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48. In still other aspects of this embodiment, a Tobacco Etch Virus protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In another aspect of this embodiment, an exogenous protease cleavage site can comprise, e.g., a Tobacco Vein Mottling Virus protease cleavage site is located within the di-chain loop of a TVEMP. In other aspects of the embodiment, an exogenous protease cleavage site can comprise, e.g., a Tobacco Vein Mottling Virus protease cleavage site located within the di-chain loop of a TVEMP comprises the consensus sequence P6-P5-V-R-F-Q*-G (SEQ ID NO: 49) or P6-P5-V-R-F-Q*-S (SEQ ID NO: 50), where P5 and P6 can be any amino acid. In other aspects of the embodiment, an exogenous protease cleavage site can comprise, e.g., a Tobacco Vein Mottling Virus protease cleavage site located within the di-chain loop of a TVEMP comprises SEQ ID NO: 51, SEQ ID NO: 52, SEQ ID NO: 53, or SEQ ID NO: 54. In still other aspects of this embodiment, a Tobacco Vein Mottling Virus protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In still another aspect of this embodiment, an exogenous protease cleavage site can comprise, e.g., a human rhinovirus 3C protease cleavage site is located within the di-chain loop of a TVEMP. In other aspects of the embodiment, an exogenous protease cleavage site can comprise, e.g., a human rhinovirus 3C protease cleavage site located within the di-chain loop of a TVEMP comprises the consensus sequence P5-P4-L-F-Q*-G-P (SEQ ID NO: 55), where P4 is G, A, V, L, I, M, S or T and P5 can any amino acid, with D or E preferred. In other aspects of the embodiment, an exogenous protease cleavage site can comprise, e.g., a human rhinovirus 3C protease cleavage site located within the di-chain loop of a TVEMP comprises SEQ ID NO: 56, SEQ ID NO: 57, SEQ ID NO: 58, SEQ ID NO: 59, SEQ ID NO: 60 or SEQ ID NO: 61. In other aspects of the embodiment, an exogenous protease cleavage site can comprise, e.g., a human rhinovirus 3C protease located within the di-chain loop of a TVEMP that can be cleaved by PRESCISSION®. In still other aspects of this embodiment, a human rhinovirus 3C protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a subtilisin cleavage site is located within the di-chain loop of a TVEMP. In other aspects of the embodiment, an exogenous protease cleavage site can comprise, e.g., a subtilisin cleavage site located within the di-chain loop of a TVEMP comprises the consensus sequence P6-P5-P4-P3-H*-Y (SEQ ID NO: 62) or P6-P5-P4-P3-Y-H* (SEQ ID NO: 63), where P3, P4 and P5 and P6 can be any amino acid. In other aspects of the embodiment, an exogenous protease cleavage site can comprise, e.g., a subtilisin cleavage site located within the di-chain loop of a TVEMP comprises SEQ ID NO: 64, SEQ ID NO: 65, or SEQ ID NO: 66. In other aspects of the embodiment, an exogenous protease cleavage site can comprise, e.g., a subtilisin cleavage site located within the di-chain loop of a TVEMP that can be cleaved by GENENASE®. In still other aspects of this embodiment, a subtilisin cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a hydroxylamine cleavage site is located within the di-chain loop of a TVEMP. In other aspects of the embodiment, an exogenous protease cleavage site can comprise, e.g., a hydroxylamine cleavage site comprising multiples of the dipeptide N*G. In other aspects of the embodiment, an exogenous protease cleavage site can comprise, e.g., a hydroxylamine cleavage site located within the di-chain loop of a TVEMP comprises SEQ ID NO: 67, or SEQ ID NO: 68. In still other aspects of this embodiment, a hydroxylamine cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a SUMO/ULP-1 protease cleavage site is located within the di-chain loop of a TVEMP. In other aspects of the embodiment, an exogenous protease cleavage site can comprise, e.g., a SUMO/ULP-1 protease cleavage site located within the di-chain loop of a TVEMP comprising the consensus sequence G-G*-P1′-P2′-P3′ (SEQ ID NO: 69), where P1′, P2′, and P3′ can be any amino acid. In other aspects of the embodiment, an exogenous protease cleavage site can comprise, e.g., a SUMO/ULP-1 protease cleavage site located within the di-chain loop of a TVEMP comprises SEQ ID NO: 70. In still other aspects of this embodiment, a SUMO/ULP-1 protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In an aspect of this embodiment, an exogenous protease cleavage site can comprise, e.g., a non-human Caspase 3 cleavage site is located within the di-chain loop of a TVEMP. In other aspects of the embodiment, an exogenous protease cleavage site can comprise, e.g., a mouse Caspase 3 protease cleavage site located within the di-chain loop of a TVEMP. In other aspects of the embodiment, an exogenous protease cleavage site can comprise, e.g., a non-human Caspase 3 protease cleavage site located within the di-chain loop of a TVEMP comprises the consensus sequence D-P3-P2-D*P1′ (SEQ ID NO: 71), where P3 can be any amino acid, with E preferred, P2 can be any amino acid and P1′ can any amino acid, with G or S preferred. In other aspects of the embodiment, an exogenous protease cleavage site can comprise, e.g., a non-human Caspase 3 protease cleavage site located within the di-chain loop of a TVEMP comprising SEQ ID NO: 72, SEQ ID NO: 73, SEQ ID NO: 74, SEQ ID NO: 75, SEQ ID NO: 76, or SEQ ID NO: 77. In still other aspects of this embodiment, a bovine enterokinase protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

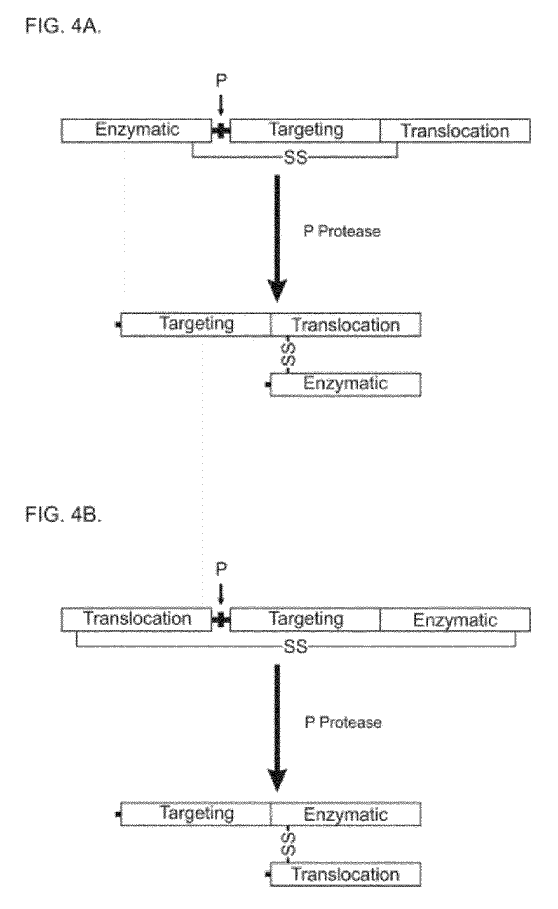

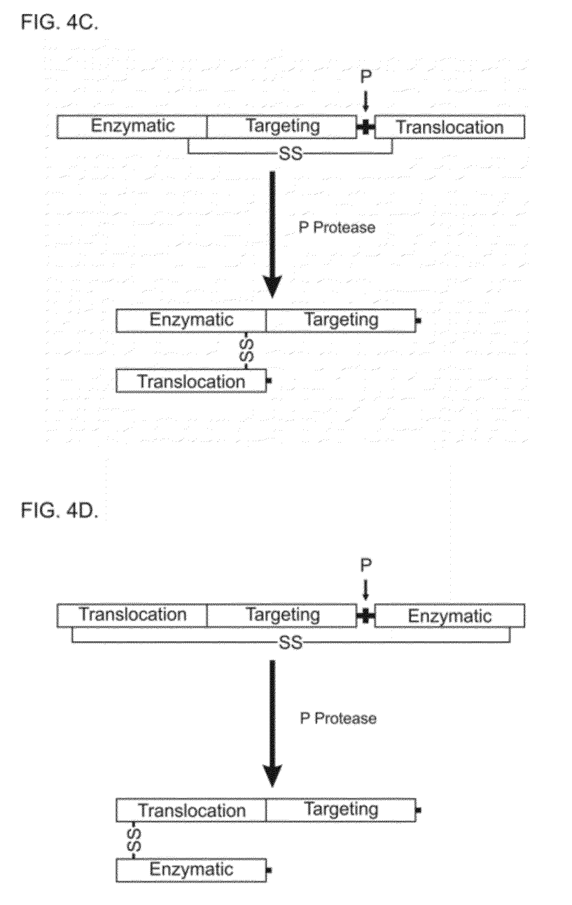

A di-chain loop region is modified to replace a naturally-occurring di-chain loop protease cleavage site for an exogenous protease cleavage site. In this modification, the naturally-occurring di-chain loop protease cleavage site is made inoperable and thus can not be cleaved by its protease. Only the exogenous protease cleavage site can be cleaved by its corresponding exogenous protease. In this type of modification, the exogenous protease site is operably-linked in-frame to a TVEMP as a fusion protein and the site can be cleaved by its respective exogenous protease. Replacement of an endogenous di-chain loop protease cleavage site with an exogenous protease cleavage site can be a substitution of the sites where the exogenous site is engineered at the position approximating the cleavage site location of the endogenous site. Replacement of an endogenous di-chain loop protease cleavage site with an exogenous protease cleavage site can be an addition of an exogenous site where the exogenous site is engineered at the position different from the cleavage site location of the endogenous site, the endogenous site being engineered to be inoperable. The location and kind of protease cleavage site may be critical because certain targeting domains require a free amino-terminal or carboxyl-terminal amino acid. For example, when a peptide targeting domain is placed between two other domains, e.g., see FIG. 4, a criterion for selection of a protease cleavage site could be whether the protease that cleaves its site leaves a flush cut, exposing the free amino-terminal or carboxyl-terminal of the targeting domain necessary for selective binding of the targeting domain to its receptor.

A naturally-occurring protease cleavage site can be made inoperable by altering at least one of the two amino acids flanking the peptide bond cleaved by the naturally-occurring di-chain loop protease. More extensive alterations can be made, with the proviso that the two cysteine residues of the di-chain loop region remain intact and the region can still form the disulfide bridge. Non-limiting examples of an amino acid alteration include deletion of an amino acid or replacement of the original amino acid with a different amino acid. Thus, in one embodiment, a naturally-occurring protease cleavage site is made inoperable by altering at least 1, 2, 3, 4, 5, 6, 7, 8, 9, 10, 15, 20 amino acids including at least one of the two amino acids flanking the peptide bond cleaved by a naturally-occurring protease. In another embodiment, a naturally-occurring protease cleavage site is made inoperable by altering at most 1, 2, 3, 4, 5, 6, 7, 8, 9, 10, 15, 20 amino acids including at least one of the two amino acids flanking the peptide bond cleaved by a naturally-occurring protease.

It is understood that a TVEMP disclosed herein can optionally further comprise a flexible region comprising a flexible spacer. A flexible region comprising flexible spacers can be used to adjust the length of a polypeptide region in order to optimize a characteristic, attribute or property of a polypeptide. As a non-limiting example, a polypeptide region comprising one or more flexible spacers in tandem can be use to better expose a protease cleavage site thereby facilitating cleavage of that site by a protease. As another non-limiting example, a polypeptide region comprising one or more flexible spacers in tandem can be use to better present a peptide targeting domain, thereby facilitating the binding of that targeting domain to its receptor.

A flexible space comprising a peptide is at least one amino acid in length and comprises non-charged amino acids with small side-chain R groups, such as, e.g., glycine, alanine, valine, leucine or serine. Thus, in an embodiment a flexible spacer can have a length of, e.g., at least 1, 2, 3, 4, 5, 6, 7, 8, 9, or 10 amino acids; or at most 1, 2, 3, 4, 5, 6, 7, 8, 9, or 10 amino acids. In still another embodiment, a flexible spacer can be, e.g., between 1-3 amino acids, between 2-4 amino acids, between 3-5 amino acids, between 4-6 amino acids, or between 5-7 amino acids. Non-limiting examples of a flexible spacer include, e.g., a G-spacers such as GGG, GGGG (SEQ ID NO: 78), and GGGGS (SEQ ID NO: 79) or an A-spacers such as AAA, AAAA (SEQ ID NO: 80) and AAAAV (SEQ ID NO: 81). Such a flexible region is operably-linked in-frame to the TVEMP as a fusion protein.

Thus, in an embodiment, a TVEMP disclosed herein can further comprise a flexible region comprising a flexible spacer. In another embodiment, a TVEMP disclosed herein can further comprise flexible region comprising a plurality of flexible spacers in tandem. In aspects of this embodiment, a flexible region can comprise in tandem, e.g., at least 1, 2, 3, 4, or 5 G-spacers; or at most 1, 2, 3, 4, or 5 G-spacers. In still other aspects of this embodiment, a flexible region can comprise in tandem, e.g., at least 1, 2, 3, 4, or 5 A-spacers; or at most 1, 2, 3, 4, or 5 A-spacers. In another aspect of this embodiment, a TVEMP can comprise a flexible region comprising one or more copies of the same flexible spacers, one or more copies of different flexible-spacer regions, or any combination thereof.

In other aspects of this embodiment, a TVEMP comprising a flexible spacer can be, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

It is envisioned that a TVEMP disclosed herein can comprise a flexible spacer in any and all locations with the proviso that TVEMP is capable of performing the intoxication process. In aspects of this embodiment, a flexible spacer is positioned between, e.g., an enzymatic domain and a translocation domain, an enzymatic domain and a peptide targeting domain, an enzymatic domain and an exogenous protease cleavage site. In other aspects of this embodiment, a G-spacer is positioned between, e.g., an enzymatic domain and a translocation domain, an enzymatic domain and a peptide targeting domain, an enzymatic domain and an exogenous protease cleavage site. In other aspects of this embodiment, an A-spacer is positioned between, e.g., an enzymatic domain and a translocation domain, an enzymatic domain and a peptide targeting domain, an enzymatic domain and an exogenous protease cleavage site.

In other aspects of this embodiment, a flexible spacer is positioned between, e.g., a peptide targeting domain and a translocation domain, a peptide targeting domain and an enzymatic domain, a peptide targeting domain and an exogenous protease cleavage site. In other aspects of this embodiment, a G-spacer is positioned between, e.g., a peptide targeting domain and a translocation domain, a peptide targeting domain and an enzymatic domain, a peptide targeting domain and an exogenous protease cleavage site. In other aspects of this embodiment, an A-spacer is positioned between, e.g., a peptide targeting domain and a translocation domain, a peptide targeting domain and an enzymatic domain, a peptide targeting domain and an exogenous protease cleavage site.

In yet other aspects of this embodiment, a flexible spacer is positioned between, e.g., a translocation domain and an enzymatic domain, a translocation domain and a peptide targeting domain, a translocation domain and an exogenous protease cleavage site. In other aspects of this embodiment, a G-spacer is positioned between, e.g., a translocation domain and an enzymatic domain, a translocation domain and a peptide targeting domain, a translocation domain and an exogenous protease cleavage site. In other aspects of this embodiment, an A-spacer is positioned between, e.g., a translocation domain and an enzymatic domain, a translocation domain and a peptide targeting domain, a translocation domain and an exogenous protease cleavage site.

It is envisioned that a TVEMP disclosed herein can comprise a peptide targeting domain in any and all locations with the proviso that TVEMP is capable of performing the intoxication process. Non-limiting examples include, locating a peptide targeting domain at the amino terminus of a TVEMP; locating a peptide targeting domain between a Clostridial toxin enzymatic domain and a translocation domain of a TVEMP; and locating a peptide targeting domain at the carboxyl terminus of a TVEMP. Other non-limiting examples include, locating a peptide targeting domain between a Clostridial toxin enzymatic domain and a Clostridial toxin translocation domain of a TVEMP. The enzymatic domain of naturally-occurring Clostridial toxins contains the native start methionine. Thus, in domain organizations where the enzymatic domain is not in the amino-terminal location an amino acid sequence comprising the start methionine should be placed in front of the amino-terminal domain. Likewise, where a peptide targeting domain is in the amino-terminal position, an amino acid sequence comprising a start methionine and a protease cleavage site may be operably-linked in situations in which a peptide targeting domain requires a free amino terminus, see, e.g., Shengwen Li et al., Degradable Clostridial Toxins, U.S. patent application Ser. No. 11/572,512 (Jan. 23, 2007), which is hereby incorporated by reference in its entirety. In addition, it is known in the art that when adding a polypeptide that is operably-linked to the amino terminus of another polypeptide comprising the start methionine that the original methionine residue can be deleted.

Thus, in an embodiment, a TVEMP can comprise an amino to carboxyl single polypeptide linear order comprising a peptide targeting domain, a translocation domain, an exogenous protease cleavage site and an enzymatic domain (FIG. 3A). In an aspect of this embodiment, a TVEMP can comprise an amino to carboxyl single polypeptide linear order comprising a peptide targeting domain, a Clostridial toxin translocation domain, an exogenous protease cleavage site and a Clostridial toxin enzymatic domain.

In another embodiment, a TVEMP can comprise an amino to carboxyl single polypeptide linear order comprising a peptide targeting domain, an enzymatic domain, an exogenous protease cleavage site, and a translocation domain (FIG. 3B). In an aspect of this embodiment, a TVEMP can comprise an amino to carboxyl single polypeptide linear order comprising a peptide targeting domain, a Clostridial toxin enzymatic domain, an exogenous protease cleavage site, a Clostridial toxin translocation domain.

In yet another embodiment, a TVEMP can comprise an amino to carboxyl single polypeptide linear order comprising an enzymatic domain, an exogenous protease cleavage site, a peptide targeting domain, and a translocation domain (FIG. 4A). In an aspect of this embodiment, a TVEMP can comprise an amino to carboxyl single polypeptide linear order comprising a Clostridial toxin enzymatic domain, an exogenous protease cleavage site, a peptide targeting domain, and a Clostridial toxin translocation domain.

In yet another embodiment, a TVEMP can comprise an amino to carboxyl single polypeptide linear order comprising a translocation domain, an exogenous protease cleavage site, a peptide targeting domain, and an enzymatic domain (FIG. 4B). In an aspect of this embodiment, a TVEMP can comprise an amino to carboxyl single polypeptide linear order comprising a Clostridial toxin translocation domain, a peptide targeting domain, an exogenous protease cleavage site and a Clostridial toxin enzymatic domain.

In another embodiment, a TVEMP can comprise an amino to carboxyl single polypeptide linear order comprising an enzymatic domain, a peptide targeting domain, an exogenous protease cleavage site, and a translocation domain (FIG. 4C). In an aspect of this embodiment, a TVEMP can comprise an amino to carboxyl single polypeptide linear order comprising a Clostridial toxin enzymatic domain, a peptide targeting domain, an exogenous protease cleavage site, a Clostridial toxin translocation domain.

In yet another embodiment, a TVEMP can comprise an amino to carboxyl single polypeptide linear order comprising a translocation domain, a peptide targeting domain, an exogenous protease cleavage site and an enzymatic domain (FIG. 4D). In an aspect of this embodiment, a TVEMP can comprise an amino to carboxyl single polypeptide linear order comprising a Clostridial toxin translocation domain, a peptide targeting domain, an exogenous protease cleavage site and a Clostridial toxin enzymatic domain.

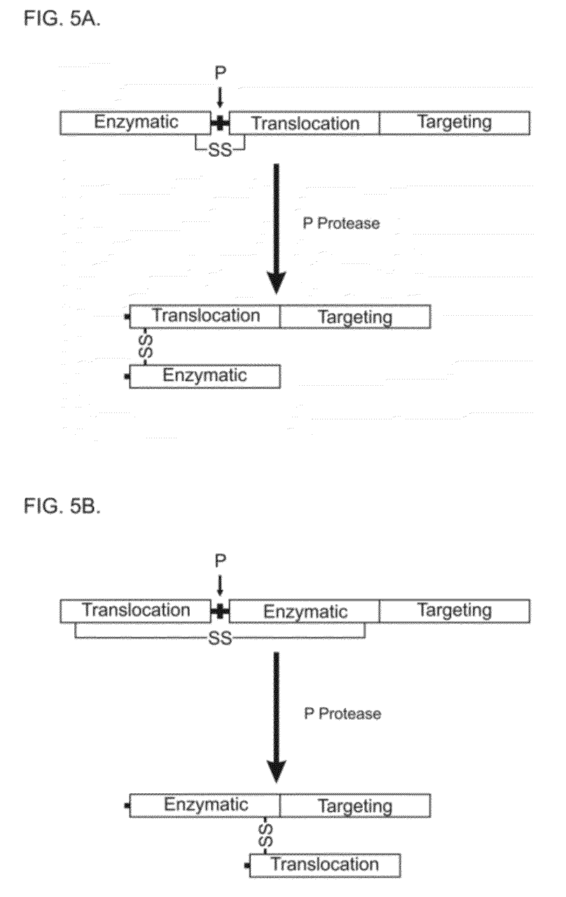

In still another embodiment, a TVEMP can comprise an amino to carboxyl single polypeptide linear order comprising an enzymatic domain, an exogenous protease cleavage site, a translocation domain, and a peptide targeting domain (FIG. 5A). In an aspect of this embodiment, a TVEMP can comprise an amino to carboxyl single polypeptide linear order comprising a Clostridial toxin enzymatic domain, an exogenous protease cleavage site, a Clostridial toxin translocation domain, and a peptide targeting domain.

In still another embodiment, a TVEMP can comprise an amino to carboxyl single polypeptide linear order comprising a translocation domain, an exogenous protease cleavage site, an enzymatic domain and a peptide targeting domain, (FIG. 5B). In an aspect of this embodiment, a TVEMP can comprise an amino to carboxyl single polypeptide linear order comprising a Clostridial toxin translocation domain, a peptide targeting domain, an exogenous protease cleavage site and a Clostridial toxin enzymatic domain.

A composition useful in the invention generally is administered as a pharmaceutical acceptable composition comprising a TVEMP. As used herein, the term “pharmaceutically acceptable” means any molecular entity or composition that does not produce an adverse, allergic or other untoward or unwanted reaction when administered to an individual. As used herein, the term “pharmaceutically acceptable composition” is synonymous with “pharmaceutical composition” and means a therapeutically effective concentration of an active ingredient, such as, e.g., any of the TVEMPs disclosed herein. A pharmaceutical composition comprising a TVEMP is useful for medical and veterinary applications. A pharmaceutical composition may be administered to a patient alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

Aspects of the present invention provide, in part, a composition comprising a TVEMP. It is envisioned that any of the composition disclosed herein can be useful in a method of treating prostate cancer, benign prostatic hyperplasia, or neovascularization or angiogenesis of a cancer in a mammal in need thereof, with the proviso that the composition prevents or reduces a symptom associated with prostate cancer, benign prostatic hyperplasia, or neovascularization or angiogenesis of a cancer. Non-limiting examples of compositions comprising a TVEMP include a TVEMP comprising a peptide targeting domain, a Clostridial toxin translocation domain and a Clostridial toxin enzymatic domain. It is envisioned that any TVEMP disclosed herein can be used, including those disclosed in, e.g., Steward, L. E. et al., Activatable Clostridial Toxins, U.S. Pat. No. 7,740,868; Dolly, J. O. et al., Activatable Clostridial Neurotoxins, U.S. Pat. No. 7,709,228; Steward, L. E. et al., Activatable Clostridial Toxins, U.S. Patent Publication 2009/0069238; Foster, K. A. et al., Fusion Proteins, US Patent Publication 2009/0035822; and Foster, K. A. et al., Non-Cytotoxic Protein Conjugates, US Patent Publication 2008/0187960; Steward, L. E. et al., Activatable Clostridial Toxins, U.S. Patent Publication 2008/0161226, each of which is incorporated by reference in its entirety. It is also understood that the two or more different TVEMPs can be provided as separate compositions or as part of a single composition.

It is also envisioned that a pharmaceutical composition comprising a TVEMP can optionally include a pharmaceutically acceptable carrier that facilitates processing of an active ingredient into pharmaceutically acceptable compositions. As used herein, the term “pharmacologically acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN′S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003). These protocols are routine procedures and any modifications are well within the scope of one skilled in the art and from the teaching herein.

It is further envisioned that a pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

In an embodiment, a composition comprising a TVEMP is a pharmaceutical composition comprising a TVEMP. In aspects of this embodiment, a pharmaceutical composition comprising a TVEMP further comprises a pharmacological carrier, a pharmaceutical component, or both a pharmacological carrier and a pharmaceutical component. In other aspects of this embodiment, a pharmaceutical composition comprising a TVEMP further comprises at least one pharmacological carrier, at least one pharmaceutical component, or at least one pharmacological carrier and at least one pharmaceutical component.

Aspects of the present invention provide, in part, a cancer. As used herein, the term “cancer” means cells exhibiting uncontrolled growth that have a pathophysiology effect. It is envisioned that the TVEMPs, compositions and methods disclosed herein can be useful to treat any cancer comprising cells that express the cognate receptor for the targeting domain present in the TVEMP. For example, a TVEMP comprising a Prostate-Specific Membrane Antigen targeting domain would be useful in treating cancer cells that express a Prostate-Specific Membrane Antigen. A cancer includes a carcinoma, a sarcoma, a lymphoma, a leukemia, a blastoma, and a germ cell tumor.

Prostate cancer is a form of cancer that develops in the prostate, a gland in the male reproductive system. Although most prostate cancers are slow growing, about one-third of prostate cancers are aggressive and fast growing. The cancer cells may metastasize from the prostate to other parts of the body, particularly the bones and lymph nodes. Prostate cancer may cause pain, difficulty in urinating, problems during sexual intercourse, or erectile dysfunction. Other symptoms can potentially develop during later stages of the disease.

Prostate cancer is classified as an adenocarcinoma, or glandular cancer, that begins when normal semen-secreting prostate gland cells mutate into cancer cells. The region of prostate gland where the adenocarcinoma is most common is the peripheral zone. Initially, small clumps of cancer cells remain confined to otherwise normal prostate glands, a condition known as carcinoma in situ or prostatic intraepithelial neoplasia (PIN). Although there is no proof that PIN is a cancer precursor, it is closely associated with cancer. Over time, these cancer cells begin to multiply and spread to the surrounding prostate tissue (the stroma) forming a tumor. Eventually, the tumor may grow large enough to invade nearby organs such as the seminal vesicles or the rectum, or the tumor cells may develop the ability to travel in the bloodstream and lymphatic system. Prostate cancer most commonly metastasizes to the bones, lymph nodes, rectum, and bladder.

An important part of evaluating prostate cancer is determining the stage, or how far the cancer has spread. The most common system is the four-stage TNM system (abbreviated from Tumor/Nodes/Metastases). Its components include the size of the tumor, the number of involved lymph nodes, and the presence of any other metastases. In the TNM system, clinical T1 and T2 cancers are found only in the prostate, while T3 and T4 cancers have spread elsewhere. Several tests can be used to look for evidence of spread. These include computed tomography to evaluate spread within the pelvis, bone scans to look for spread to the bones, and endorectal coil magnetic resonance imaging to closely evaluate the prostatic capsule and the seminal vesicles. Bone scans should reveal osteoblastic appearance due to increased bone density in the areas of bone metastasis—opposite to what is found in many other cancers that metastasize.

Aspects of the present invention provide, in part, reducing a symptom associated with cancer. In an aspect, the symptom reduced is an increase in the growth rate of cancer cells. In another aspect, the symptom reduced is an increase in the cell division rate of cancer cells. In yet another aspect, the symptom reduced is an increase in the extent of invasion of cancer cells into adjacent tissue or organs. In still another aspect, the symptom reduced is an increase in the extent of metastasis. In a further aspect, the symptom reduced is an increase in angiogenesis. In a yet further aspect, the symptom reduced is a decrease in apoptosis. In a still further aspect, the symptom reduced is a decrease in cell death or cell necrosis. In another aspect, the symptom reduced is pain, difficulty in urinating, problems during sexual intercourse, or erectile dysfunction. In yet another aspect, the symptom reduced is a decrease in urinary frequency, a decrease in urinary urgency, a decrease in urgency incontinence, or a decrease in nocturia. In still another aspect, the symptom reduced is a decrease in urinary streaming, a decrease in urinary hesitancy, a decrease in urgency intermittency, or a decrease in urinary straining, or a decrease in urinary dribbling. Thus, a TVEMP treatment will decrease the growth rate of cancer cells, decrease the cell division rate of cancer cells, decrease the extent of invasion of cancer cells into adjacent tissue or organs, decrease the extent of metastasis, decrease angiogenesis, increase apoptosis, and/or increase cell death and/or cell necrosis.

Aspects of the present invention provide, in part, a disease of hyperproliferation. As used herein, the term “disease of hyperproliferation” means cells exhibiting uncontrolled cell division and/or growth that have a pathophysiology effect. It is envisioned that the TVEMPs, compositions and methods disclosed herein can be useful to treat any disease of hyperproliferation comprising cells that express the cognate receptor for the targeting domain present in the TVEMP. For example, a TVEMP comprising a Prostate-Specific Membrane Antigen targeting domain would be useful in treating hyperproliferating cells that express a Prostate-Specific Membrane Antigen. A disease of hyperproliferation include BPH and a benign tumor.

Benign prostatic hyperplasia (BPH) is characterized by hyperplasia of prostatic stromal and epithelial cells, resulting in the formation of large, fairly discrete nodules in the periurethral region of the prostate. When sufficiently large, the nodules compress the urethral canal to cause partial, or sometimes virtually complete, obstruction of the urethra, which interferes the normal flow of urine. It leads to symptoms of urinary hesitancy, frequent urination, dysuria (painful urination), increased risk of urinary tract infections, and urinary retention.

Benign prostatic hyperplasia symptoms are classified as storage or voiding. Storage symptoms include urinary frequency, urgency (compelling need to void that cannot be deferred), urgency incontinence, and voiding at night (nocturia). Voiding symptoms include urinary stream, hesitancy (needing to wait for the stream to begin), intermittency (when the stream starts and stops intermittently), straining to void, and dribbling. Pain and dysuria are usually not present. These storage and voiding symptoms are evaluated using the International Prostate Symptom Score (IPSS) questionnaire, designed to assess the severity of BPH.

BPH can be a progressive disease, especially if left untreated. Incomplete voiding results in stasis of bacteria in the bladder residue and an increased risk of urinary tract infection. Urinary bladder stones are formed from the crystallization of salts in the residual urine. Urinary retention, termed acute or chronic, is another form of progression. Acute urinary retention is the inability to void, while in chronic urinary retention the residual urinary volume gradually increases, and the bladder distends. Some patients that suffer from chronic urinary retention may eventually progress to renal failure, a condition termed obstructive uropathy.

Aspects of the present invention provide, in part, reducing a symptom associated with a disease of hyperproliferation. In an aspect, the symptom reduced is an increase in the growth rate of hyperproliferating cells. In another aspect, the symptom reduced is an increase in the cell division rate of hyperproliferating cells. In yet another aspect, the symptom reduced is a decrease in the extent that a disease of hyperproliferation becomes a tumor. In still another aspect, the symptom reduced is an increase in angiogenesis. In a further aspect, the symptom reduced is a decrease in apoptosis. In a yet further aspect, the symptom reduced is a decrease in cell death or cell necrosis. Thus, a TVEMP treatment will decrease the growth rate of hyperproliferating cells, decrease the cell division rate of hyperproliferating cells, decrease the extent to which a disease of hyperproliferation becomes a tumor, decrease angiogenesis, increase apoptosis, and/or increase cell death and/or cell necrosis.

Aspects of the present invention provide, in part, a neovascularization or angiogenesis associated with a cancer. As used herein, the term “neovascularization or angiogenesis associated with a cancer” means cells exhibiting uncontrolled cell division and/or growth that have a pathophysiology effect where formation of new blood vessels is associated with effect. It is envisioned that the TVEMPs, compositions and methods disclosed herein can be useful to treat any disease of neovascularization or angiogenesis associated with a cancer comprising cells that express the cognate receptor for the targeting domain present in the TVEMP. For example, a TVEMP comprising a Prostate-Specific Membrane Antigen targeting domain would be useful in treating proliferating or migrating endothelial or endothelial progenitor cells that express a Prostate-Specific Membrane Antigen. A neovascularization or angiogenesis associated with a cancer include a gastric cancer, such as, e.g., a gastric adenocarcinoma or a gastric carcinoma, a colorectal cancer, such as, e.g., a colorectal adenocarcinoma or a colorectal carcinoma, and metastatic cancer originating from these sites.

Neovascularization or angiogenesis associated with a cancer refers to any tumor that requires or promotes new blood vessel formation as part of its disease development. As cancer cells proliferate within a tumor they also face other challenges like the limited supply of oxygen and nutrients that would induce apoptosis. In order to sustain cell proliferation and tumor growth a cancer needs to stimulate the growth of existing blood vessels as well as initiate the growth of new blood vessels. This is accomplished, in part, by the secretion of pro-angiogenic factors by cancer cells in order to stimulate endothelial cells to begin growing blood vessels. In addition, pro-angiogenic factors sequestered in the extracellular matrix can be released by digestion of the matrix performed by proteases secreted by tumor cells. Inhibition of angiogenesis is a validated therapeutic target as several approved drugs target this pathway as a treatment for cancer and other pro-angiogenesis diseases.

Aspects of the present invention provide, in part, reducing a symptom associated with a neovascularization or angiogenesis associated with a cancer. In an aspect, the symptom reduced is an increase in the growth rate of endothelial or endothelial progenitor cells associated with neovascularization. In another aspect, the symptom reduced is an increase in the cell division rate of endothelial or endothelial progenitor cells associated with neovascularization. In another aspect, the symptom reduced is an increase in migration of endothelial or endothelial progenitor cells associated with neovascularization. In another aspect, the symptom reduced is an increase in survival of endothelial or endothelial progenitor cells associated with neovascularization. In yet another aspect, the symptom reduced is an increase in the extent of invasion of cancer cells associated with neovascularization into adjacent tissue or organs. In still another aspect, the symptom reduced is an increase in the extent of metastasis of cancer cells associated with neovascularization. In a further aspect, the symptom reduced is an increase in angiogenesis or neovascularization. In a yet further aspect, the symptom reduced is a decrease in apoptosis of cancer cells associated with neovascularization. In a still further aspect, the symptom reduced is a decrease in cell death or cell necrosis of cancer cells associated with neovascularization. Thus, a TVEMP treatment will decrease the growth rate of cancer cells associated with neovascularization, decrease the cell division rate of cancer cells associated with neovascularization, decrease the extent of invasion of cancer cells associated with neovascularization into adjacent tissue or organs, decrease the extent of metastasis of cancer cells associated with neovascularization, decrease proliferation, migration, and survival of endothelial or endothelial progenitor cells associated with neovascularization, decrease angiogenesis, increase apoptosis of cancer cells associated with neovascularization, and/or increase cell death and/or cell necrosis of cancer cells associated with neovascularization.

Aspects of the present invention provide, in part, a mammal. A mammal includes a human, and a human can be a patient. Other aspects of the present invention provide, in part, an individual. An individual includes a human, and a human can be a patient.

Aspects of the present invention provide, in part, administering a composition comprising a TVEMP. As used herein, the term “administering” means any delivery mechanism that provides a composition comprising a TVEMP to a patient that potentially results in a clinically, therapeutically, or experimentally beneficial result. A TVEMP can be delivered to a patient using a cellular uptake approach where a TVEMP is delivered intracellular or a gene therapy approach where a TVEMP is express derived from precursor RNAs expressed from an expression vectors.

A composition comprising a TVEMP as disclosed herein can be administered to a mammal using a cellular uptake approach. Administration of a composition comprising a TVEMP using a cellular uptake approach comprise a variety of enteral or parenteral approaches including, without limitation, oral administration in any acceptable form, such as, e.g., tablet, liquid, capsule, powder, or the like; topical administration in any acceptable form, such as, e.g., drops, spray, creams, gels or ointments; intravascular administration in any acceptable form, such as, e.g., intravenous bolus injection, intravenous infusion, intra-arterial bolus injection, intra-arterial infusion and catheter instillation into the vasculature; peri- and intra-tissue administration in any acceptable form, such as, e.g., intraperitoneal injection, intramuscular injection, subcutaneous injection, subcutaneous infusion, intraocular injection, retinal injection, or sub-retinal injection or epidural injection; intravesicular administration in any acceptable form, such as, e.g., catheter instillation; and by placement device, such as, e.g., an implant, a patch, a pellet, a catheter, an osmotic pump, a suppository, a bioerodible delivery system, a non-bioerodible delivery system or another implanted extended or slow release system. An exemplary list of biodegradable polymers and methods of use are described in, e.g., Handbook of Biodegradable Polymers (Abraham J. Domb et al., eds., Overseas Publishers Association, 1997).

A composition comprising a TVEMP can be administered to a mammal by a variety of methods known to those of skill in the art, including, but not restricted to, encapsulation in liposomes, by ionophoresis, or by incorporation into other vehicles, such as hydrogels, cyclodextrins, biodegradable nanocapsules, and bioadhesive microspheres, or by proteinaceous vectors. Delivery mechanisms for administering a composition comprising a TVEMP to a patient are described in, e.g., Leonid Beigelman et al., Compositions for the Delivery of Negatively Charged Molecules, U.S. Pat. No. 6,395,713; and Achim Aigner, Delivery Systems for the Direct Application of siRNAs to Induce RNA Interference (RNAi)in vivo, 2006(716559) J. Biomed. Biotech. 1-15 (2006); Controlled Drug Delivery: Designing Technologies for the Future (Kinam Park & Randy J. Mrsny eds., American Chemical Association, 2000); Vernon G. Wong & Mae W. L. Hu, Methods for Treating Inflammation-mediated Conditions of the Eye, U.S. Pat. No. 6,726,918; David A. Weber et al., Methods and Apparatus for Delivery of Ocular Implants, U.S. Patent Publication No. US2004/0054374; Thierry Nivaggioli et al., Biodegradable Ocular Implant, U.S. Patent Publication No. US2004/0137059; Patrick M. Hughes et al., Anti-Angiogenic Sustained Release Intraocular Implants and Related Methods, U.S. patent application Ser. No. 11/364,687; and Patrick M. Hughes et al., Sustained Release Intraocular Drug Delivery Systems, U.S. Patent Publication 2006/0182783, each of which is hereby incorporated by reference in its entirety.

A composition comprising a TVEMP as disclosed herein can also be administered to a patient using a gene therapy. A TVEMP can be expressed from nucleic acid molecules operably-linked to an expression vector, see, e.g., P. D. Good et al., Expression of Small, Therapeutic RNAs in Human Cell Nuclei, 4(1) Gene Ther. 45-54 (1997); James D. Thompson, Polymerase III-based expression of therapeutic RNAs, U.S. Pat. No. 6,852,535 (Feb. 8, 2005); Maciej Wiznerowicz et al., Tuning Silence: Conditional Systems for RNA Interference, 3(9) Nat. Methods 682-688m (2006); Ola Snøve and John J. Rossi, Expressing Short Hairpin RNAi in vivo, 3(9) Nat. Methods 689-698 (2006); and Charles X. Li et al., Delivery of RNA Interference, 5(18) Cell Cycle 2103-2109 (2006). A person of ordinary skill in the art would realize that any TVEMP can be expressed in eukaryotic cells using an appropriate expression vector.

Expression vectors capable of expressing a TVEMP can provide persistent or stable expression of the TVEMP in a cell manifesting a symptom associated with a cancer. Alternatively, expression vectors capable of expressing a TVEMP can provide for transient expression of the TVEMP in a cell manifesting a symptom associated with a cancer. Such transiently expressing vectors can be repeatedly administered as necessary. A TVEMP-expressing vectors can be administered by a delivery mechanism and route of administration discussed above, by administration to target cells ex-planted from a patient followed by reintroduction into the patient, or by any other means that would allow for introduction into the desired target cell, see, e.g., Larry A. Couture and Dan T. Stinchcomb, Anti-gene Therapy: The Use of Ribozymes to Inhibit Gene Function, 12(12) Trends Genet. 510-515 (1996).

The actual delivery mechanism used to administer a composition comprising a TVEMP to a mammal can be determined by a person of ordinary skill in the art by taking into account factors, including, without limitation, the type of cancer or disease of hyperproliferation, or pathological angiogenesis; the location of the cancer or disease of hyperproliferation, or pathological angiogenesis; the cause of the cancer or disease of hyperproliferation, or pathological angiogenesis; the severity of the cancer or disease of hyperproliferation, or pathological angiogenesis; the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the patient, such as, e.g., age, weight, general health and the like, or any combination thereof.

In an embodiment, a composition comprising a TVEMP is administered to the site to be treated by injection. In aspects of this embodiment, injection of a composition comprising a TVEMP is by, e.g., intramuscular injection, intraorgan injection, subdermal injection, dermal injection, or injection into any other body area for the effective administration of a composition comprising a TVEMP. In aspects of this embodiment, injection of a composition comprising a TVEMP is a tumor or into the area surrounding the tumor. In other aspects of this embodiment, injection of a composition comprising a TVEMP is in a region comprising a disease of hyperproliferation or into the area surrounding a disease of hyperproliferation. In other aspects of this embodiment, injection of a composition comprising a TVEMP is in a region comprising pathological angiogenesis or into the area surrounding pathological angiogenesis.

A composition comprising a TVEMP can be administered to a mammal using a variety of routes. Routes of administration suitable for a method of treating a cancer or disease of hyperproliferation or pathological angiogenesis as disclosed herein include both local and systemic administration. Local administration results in significantly more delivery of a composition to a specific location as compared to the entire body of the mammal, whereas, systemic administration results in delivery of a composition to essentially the entire body of the patient. Routes of administration suitable for a method of treating a cancer or disease of hyperproliferation or pathological angiogenesis as disclosed herein also include both central and peripheral administration. Central administration results in delivery of a composition to essentially the central nervous system of the patient and includes, e.g., intrathecal administration, epidural administration as well as a cranial injection or implant. Peripheral administration results in delivery of a composition to essentially any area of a patient outside of the central nervous system and encompasses any route of administration other than direct administration to the spine or brain. The actual route of administration of a composition comprising a TVEMP used in a mammal can be determined by a person of ordinary skill in the art by taking into account factors, including, without limitation, the type of cancer or disease of hyperproliferation or pathological angiogenesis, the location of the cancer or disease of hyperproliferation or pathological angiogenesis, the cause of the cancer or disease of hyperproliferation or pathological angiogenesis, the severity of the cancer or disease of hyperproliferation or pathological angiogenesis, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the mammal, such as, e.g., age, weight, general health and the like, or any combination thereof.

In an embodiment, a composition comprising a TVEMP is administered systemically to a mammal. In another embodiment, a composition comprising a TVEMP is administered locally to a mammal. In an aspect of this embodiment, a composition comprising a TVEMP is administered to a tumor of a mammal. In another aspect of this embodiment, a composition comprising a TVEMP is administered to the area surrounding a tumor of a mammal. In yet another aspect of this embodiment, a composition comprising a TVEMP is administered to a region comprising a disease of hyperproliferation of a mammal. In still another aspect of this embodiment, a composition comprising a TVEMP is administered to the area surrounding a disease of hyperproliferation of a mammal. In yet another aspect of this embodiment, a composition comprising a TVEMP is administered to a region comprising a pathological angiogenesis of a mammal. In still another aspect of this embodiment, a composition comprising a TVEMP is administered to the area surrounding pathological angiogenesis of a mammal.

Aspects of the present invention provide, in part, administering a therapeutically effective amount of a composition comprising a TVEMP. As used herein, the term “therapeutically effective amount” is synonymous with “therapeutically effective dose” and when used in reference to treating a cancer or disease of hyperproliferation or pathological angiogenesis means the minimum dose of a TVEMP necessary to achieve the desired therapeutic effect and includes a dose sufficient to reduce a symptom associated with a cancer or disease of hyperproliferation or pathological angiogenesis. In aspects of this embodiment, a therapeutically effective amount of a composition comprising a TVEMP reduces a symptom associated with a cancer or disease of hyperproliferation or pathological angiogenesis by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a TVEMP reduces a symptom associated with a cancer or disease of hyperproliferation or pathological angiogenesis by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a TVEMP reduces a symptom associated with a cancer or disease of hyperproliferation or pathological angiogenesis by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of the TVEMP is the dosage sufficient to reduces a symptom associated with a cancer or disease of hyperproliferation or pathological angiogenesis for, e.g., at least one week, at least one month, at least two months, at least three months, at least four months, at least five months, at least six months, at least seven months, at least eight months, at least nine months, at least ten months, at least eleven months, or at least twelve months.

The actual therapeutically effective amount of a composition comprising a TVEMP to be administered to a mammal can be determined by a person of ordinary skill in the art by taking into account factors, including, without limitation, the type of cancer or disease of hyperproliferation or pathological angiogenesis, the location of the cancer or disease of hyperproliferation or pathological angiogenesis, the cause of the cancer or disease of hyperproliferation or pathological angiogenesis, the severity of the cancer or disease of hyperproliferation or pathological angiogenesis, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the patient, such as, e.g., age, weight, general health and the like, or any combination thereof. Additionally, where repeated administration of a composition comprising a TVEMP is used, the actual effect amount of a composition comprising a TVEMP will further depend upon factors, including, without limitation, the frequency of administration, the half-life of the composition comprising a TVEMP, or any combination thereof. In is known by a person of ordinary skill in the art that an effective amount of a composition comprising a TVEMP can be extrapolated from in vitro assays and in vivo administration studies using animal models prior to administration to humans. Wide variations in the necessary effective amount are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors.

As a non-limiting example, when administering a composition comprising a TVEMP to a mammal, a therapeutically effective amount generally is in the range of about 1 fg to about 3.0 mg. In aspects of this embodiment, an effective amount of a composition comprising a TVEMP can be, e.g., about 100 fg to about 3.0 mg, about 100 pg to about 3.0 mg, about 100 ng to about 3.0 mg, or about 100 μg to about 3.0 mg. In other aspects of this embodiment, an effective amount of a composition comprising a TVEMP can be, e.g., about 100 fg to about 750 μg, about 100 pg to about 750 μg, about 100 ng to about 750 μg, or about 1 μg to about 750 μg. In yet other aspects of this embodiment, a therapeutically effective amount of a composition comprising a TVEMP can be, e.g., at least 1 fg, at least 250 fg, at least 500 fg, at least 750 fg, at least 1 pg, at least 250 pg, at least 500 pg, at least 750 pg, at least 1 ng, at least 250 ng, at least 500 ng, at least 750 ng, at least 1 μg, at least 250 μg, at least 500 μg, at least 750 μg, or at least 1 mg. In still other aspects of this embodiment, a therapeutically effective amount of a composition comprising a TVEMP can be, e.g., at most 1 fg, at most 250 fg, at most 500 fg, at most 750 fg, at most 1 pg, at most 250 pg, at most 500 pg, at most 750 pg, at most 1 ng, at most 250 ng, at most 500 ng, at most 750 ng, at most 1 μg, at least 250 μg, at most 500 μg, at most 750 μg, or at most 1 mg.

As another non-limiting example, when administering a composition comprising a TVEMP to a mammal, a therapeutically effective amount generally is in the range of about 0.00001 mg/kg to about 3.0 mg/kg. In aspects of this embodiment, an effective amount of a composition comprising a TVEMP can be, e.g., about 0.0001 mg/kg to about 0.001 mg/kg, about 0.03 mg/kg to about 3.0 mg/kg, about 0.1 mg/kg to about 3.0 mg/kg, or about 0.3 mg/kg to about 3.0 mg/kg. In yet other aspects of this embodiment, a therapeutically effective amount of a composition comprising a TVEMP can be, e.g., at least 0.00001 mg/kg, at least 0.0001 mg/kg, at least 0.001 mg/kg, at least 0.01 mg/kg, at least 0.1 mg/kg, or at least 1 mg/kg. In yet other aspects of this embodiment, a therapeutically effective amount of a composition comprising a TVEMP can be, e.g., at most 0.00001 mg/kg, at most 0.0001 mg/kg, at most 0.001 mg/kg, at most 0.01 mg/kg, at most 0.1 mg/kg, or at most 1 mg/kg.

Dosing can be single dosage or cumulative (serial dosing), and can be readily determined by one skilled in the art. For instance, treatment of a cancer may comprise a one-time administration of an effective dose of a composition comprising a TVEMP. As a non-limiting example, an effective dose of a composition comprising a TVEMP can be administered once to a patient, e.g., as a single injection or deposition at or near the site exhibiting a symptom of a cancer. Alternatively, treatment of a cancer may comprise multiple administrations of an effective dose of a composition comprising a TVEMP carried out over a range of time periods, such as, e.g., daily, once every few days, weekly, monthly or yearly. As a non-limiting example, a composition comprising a TVEMP can be administered once or twice yearly to a mammal. The timing of administration can vary from mammal to mammal, depending upon such factors as the severity of a mammal's symptoms. For example, an effective dose of a composition comprising a TVEMP can be administered to a mammal once a month for an indefinite period of time, or until the patient no longer requires therapy. A person of ordinary skill in the art will recognize that the condition of the mammal can be monitored throughout the course of treatment and that the effective amount of a composition comprising a TVEMP that is administered can be adjusted accordingly.

A composition comprising a TVEMP as disclosed herein can also be administered to a mammal in combination with other therapeutic compounds to increase the overall therapeutic effect of the treatment. The use of multiple compounds to treat an indication can increase the beneficial effects while reducing the presence of side effects.

Aspects of the present invention can also be described as follows:
1. A TVEMP comprising a Prostate-Specific Membrane Antigen targeting domain, a Clostridial toxin translocation domain and a Clostridial toxin enzymatic domain.
2. A TVEMP comprising a Prostate-Specific Membrane Antigen targeting domain, a Clostridial toxin translocation domain and a Clostridial toxin enzymatic domain, and an exogenous protease cleavage site.
3. The TVEMP of embodiment 1, wherein the TVEMP comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.
4. The TVEMP of embodiment 2, wherein the TVEMP comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.
5. The TVEMP of embodiments 1-4, wherein the Prostate-Specific Membrane Antigen targeting domain comprises SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112.
6. The TVEMP of embodiments 1-5, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
7. The TVEMP of embodiments 1-6, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
8. The TVEMP of embodiments 2 and 4-7, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.
9. A composition comprising a TVEMP of embodiments 1-8.
10. The composition of embodiment 9, wherein the composition is a pharmaceutical composition.
11. The composition of embodiment 10, wherein the pharmaceutical composition comprises a pharmaceutical carrier, pharmaceutical excipient, or any combination thereof.
12. A method of treating a prostate cancer in a mammal, the method comprising the step of administering to the mammal in need thereof a therapeutically effective amount of a composition including a TVEMP of embodiments 1-11, wherein administration of the composition reduces a symptom associated with the prostate cancer.
13. A use of a TVEMP for the treatment of a prostate cancer in a mammal in need thereof, the use comprising the step of administering to the mammal a therapeutically effective amount of a composition including a TVEMP of embodiments 1-11, wherein administration of the TVEMP reduces a symptom associated with the prostate cancer.
14. The method of embodiment 12 or use of embodiment 13, wherein the prostate cancer is a prostatic epithelial cancer, a prostatic intraepithelial neoplasia, or a prostatic adenocarcinoma.
15. A method of treating a benign prostatic hyperplasia in a mammal, the method comprising the step of administering to the mammal in need thereof a therapeutically effective amount of a composition including a TVEMP of embodiments 1-11, wherein administration of the composition reduces a symptom associated with the benign prostatic hyperplasia.
16. A use of a TVEMP for the treatment of a benign prostatic hyperplasia in a mammal in need thereof, the use comprising the step of administering to the mammal a therapeutically effective amount of a composition including a TVEMP of embodiments 1-11, wherein administration of the TVEMP reduces a symptom associated with the benign prostatic hyperplasia.
17. A method of treating neovascularization or pathological angiogenesis associated with a cancer in a mammal, the method comprising the step of administering to the mammal in need thereof a therapeutically effective amount of a composition including a TVEMP of embodiments 1-11, wherein administration of the composition reduces a symptom associated with the neovascularization or pathological angiogenesis associated with a cancer.
18. A use of a TVEMP for the treatment of neovascularization or pathological angiogenesis associated with a cancer in a mammal in need thereof, the use comprising the step of administering to the mammal a therapeutically effective amount of a composition including a TVEMP of embodiments 1-11, wherein administration of the TVEMP reduces a symptom associated with the neovascularization or pathological angiogenesis associated with a cancer.
19. The method of embodiment 15 or use of embodiment 16, wherein the neovascularization or pathological angiogenesis associated with a cancer is a gastric cancer or a colorectal cancer.
20. The method or use of embodiment 19, wherein the gastric cancer is a gastric adenocarcinoma, a gastric carcinoma, or a metastatic cancer originating from a gastric adenocarcinoma or a gastric carcinoma.
21. The method or use of embodiment 19, wherein the colorectal cancer is a colorectal adenocarcinoma, a colorectal carcinoma, or a metastatic cancer originating from a colorectal adenocarcinoma or a colorectal carcinoma.
22. A use of a TVEMP in the manufacturing a medicament for treating a prostate cancer in a mammal in need thereof, wherein the TVEMP comprises a targeting domain, a Clostridial toxin translocation domain and a Clostridial toxin enzymatic domain.
23. A use of a TVEMP in the manufacturing a medicament for treating a benign prostatic hyperplasia in a mammal in need thereof, wherein the TVEMP comprises a Prostate-Specific Membrane Antigen targeting domain, a Clostridial toxin translocation domain and a Clostridial toxin enzymatic domain.
24. A use of a TVEMP in the manufacturing a medicament for treating neovascularization or pathological angiogenesis associated with a cancer in a mammal in need thereof, wherein the TVEMP comprises a targeting domain, a Clostridial toxin translocation domain and a Clostridial toxin enzymatic domain.
25. The use of embodiments 22-24, wherein the TVEMP comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.
26. The use of embodiments 22-24, wherein the TVEMP further comprises an exogenous protease cleavage site.
27. The use of embodiment 26, wherein the TVEMP comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.
27. The use of embodiments 22-26, wherein the Prostate-Specific Membrane Antigen targeting domain comprises SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112.
28. The use of embodiments 22-27, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
29. The use of embodiments 22-28, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
30. The use of embodiments 26-29, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.
31. A use of embodiments 22-30, wherein the TVEMP is combined into a composition.
32. The use of embodiment 31, wherein the composition is a pharmaceutical composition.
33. The use of embodiment 32, wherein the pharmaceutical composition further comprises a pharmaceutical carrier, pharmaceutical excipient, or any combination thereof.

### EXAMPLES

The following examples illustrate representative embodiments now contemplated, but should not be construed to limit the disclosed TVEMPs, compositions including TVEMPs, and methods of treating a prostate cancer, a benign prostatic hyperplasia, and/or a neovascular-associated cancer using such compositions.

### Example 1

### Light Chain Assays

This example illustrates how to screen cancer cells in order to determine which Clostridial toxin light chain had an effect sufficient to provide a therapeutic benefit in a cancer treatment.

To identify which Clostridial toxin light chain or active fragment thereof was useful in making a TVEMP for treating a cancer using a method disclosed herein, a Clostridial toxin light chain cleavage assay was conducted. These assays address two fundamental issues. First, the light chains of the various botulinum neurotoxin serotypes cleave different SNARE substrates. In addition, some cells may only express SNAP-23 which is not cleavable by naturally-occurring botulinum neurotoxins. These cells would not be sensitive to LC/A, but may be sensitive to LC/B and LC/C1 if they express synaptobrevin-2 (VAMP-2) and/or Syntaxin, respectively. Second, this transfection assay allows the examination of the cellular effects of the light chains on cancer cells in a way that is independent of receptor binding and translocation into the cell. Taken together, this assay allows the examination of the effects of cleaving SNARE proteins on a variety of cancer cell lines encompassing several types of human cancers.

Mammalian expression constructs encoding a fusion protein comprising a green fluorescent protein (GFP) linked to a light chain of different botulinum neurotoxin serotypes were made using standard procedures. These expression constructs were designated 1) pQBI25/GFP, a construct expressing GFP of SEQ ID NO: 113 encoded by the polynucleotide of SEQ ID NO: 114; 2) pQBI25/GFP-LC/A, a construct expressing GFP-LC/A fusion protein of SEQ ID NO: 115 encoded by the polynucleotide of SEQ ID NO: 116; 3) pQBI/GFP-LC/B, a construct expressing GFP-LC/B fusion protein of SEQ ID NO: 117 encoded by the polynucleotide of SEQ ID NO: 118; 4) pQBI/GFP-LC/C1, a construct expressing GFP-LC/C1 fusion protein of SEQ ID NO: 119 encoded by the polynucleotide of SEQ ID NO: 120; and 5) pQBI/GFP-LC/E, a construct expressing GFP-LC/E fusion protein of SEQ ID NO: 121 encoded by the polynucleotide of SEQ ID NO: 122. The light chains for these particular botulinum toxin serotypes were selected because overall, the light chains cleave one of the three predominant SNARE proteins SNAP-25, VAMP, or Syntaxin.

To culture cells, an appropriate density of cells were plated into the wells of 6-well tissue culture plates containing 3 mL of an appropriate medium (Table 5). The cells were grown in a 37° C. incubator under 5% carbon dioxide until cells reached the appropriate density (about 1×10⁶ cells). A 500 μL transfection solution was prepared by adding 250 μL of OPTI-MEM Reduced Serum Medium containing 10 μL of LipofectAmine 2000 (Invitrogen Inc., Carlsbad, Calif.), incubated at room temperature for 5 minutes, to 250 μL of OPTI-MEM Reduced Serum Medium containing 5 μg of the desired mammalian expression construct. This transfection mixture was incubated at room temperature for approximately 25 minutes. The growth media was replaced with fresh unsupplemented serum-free media and the 500 μL transfection solution was added to the cells. The cells were then incubated in a 37° C. incubator under 5% carbon dioxide for approximately 8 hours. The transfection media was replaced with fresh unsupplemented serum-free media and the cells then incubated in a 37° C. incubator under 5% carbon dioxide for approximately 48 hours. After this incubation, the cells were washed by aspirating the media and rinsing each well with 3 mL of 1×PBS.

TABLE 5
Cell Lines and Media

| Cell Line | Origin | Source | Serum Growth Media Composition |
|---|---|---|---|
| RT4 | Human urinary | ATCC HTB-2 | McCoy's 5a media with 10% fetal bovine |
| | bladder transitional | | serum, 100 U/mL Penicillin, and 100 μg/mL |
| | cell carcinoma | | Streptomycin |
| P19 | Mouse embryonic | ATCC CRL-1825 | Alpha Minimal Essential Medium media |
| | carcinoma | | with 7.5% bovine calf serum, 2.5% fetal |
| | | | bovine calf serum, 100 U/mL Penicillin, and |
| | | | 100 μg/mL Streptomycin |
| NCI H69 | Human small lung | ATCC HTB-119 | RPMI-1640 media with 10% fetal bovine |
| | carcinoma | | serum, 100 U/mL Penicillin, and 100 μg/mL |
| | | | Streptomycin |
| NCI H82 | Human small lung | ATCC HTB-175 | RPMI-1640 media with 10% fetal bovine |
| | carcinoma | | serum, 100 U/mL Penicillin, and 100 μg/mL |
| | | | Streptomycin |
| DU-145 | Human prostate | ATCC HTB-81 | Eagle's Minimum Essential Medium with 10% |
| | carcinoma derived | | fetal bovine serum, 100 U/mL Penicillin, |
| | from brain | | and 100 μg/mL Streptomycin |
| T24 | Human urinary | ATCC HTB-4 | McCoy's 5a media with 10% fetal bovine |
| | bladder transitional | | serum, 100 U/mL Penicillin, and 100 μg/mL |
| | cell carcinoma | | Streptomycin |
| J82 | Human urinary | ATCC HTB-1 | Eagle's Minimum Essential Medium with 10% |
| | bladder transitional | | fetal bovine serum, 100 U/mL Penicillin, |
| | cell carcinoma | | and 100 μg/mL Streptomycin |
| HIT-T15 | Syrian Golden | ATCC CRL-1777 | Eagle's Minimum Essential Medium (low |
| | Hamster, pancreatic | | glucose) with 10% fetal bovine serum, 100 |
| | islet of Langerhans | | U/mL Penicillin, and 100 μg/mL |
| | beta cells | | Streptomycin |

The cells were first analyzed using fluorescent microscopy for the expression of GFP, which also indicated the simultaneous expression of the attached light chain. To detect the expression and subcellular localization of the GFP-LC fusion proteins, the cells were examined by confocal microscopy. Cells from the cell lines RT4, P19, NCl H69, NCl H82, DU145, T24, and J82, transfected and washed as described above, were fixed with 4% paraformaldehyde. The fixed cells were imaged with a confocal microscope using a 488 nm excitation laser and an emission path of 510-530 nm. The data shows that each cell type was successfully transfected and, that except the small cell lung cancer cell lines NCl H69 and NCl H82, cells from each cell line expressed both GFP and the GFP-light chain fusion proteins (Table 6).

TABLE 6
Expression of Mammalian Constructs in Cells

| | | Expression | | | | |
|---|---|---|---|---|---|---|
| | | | GFP- | GFP- | GFP- | |
| Cell Line | Origin | GFP | LC/A | LC/B | LC/C1 | GFP-LC/E |
| RT4 | Bladder | + | + | + | + | + |
| | carcinoma | | | | | |
| P19 | Embryonic | + | + | + | + | + |
| | carcinoma | | | | | |
| NCI H69 | Small Cell Lung | − | − | − | − | − |
| | carcinoma | | | | | |
| NCI H82 | Small Cell Lung | − | − | − | − | − |
| | carcinoma | | | | | |
| DU145 | Prostate | + | + | + | + | + |
| | carcinoma | | | | | |
| T24 | Bladder | + | + | + | + | + |
| | carcinoma | | | | | |
| J82 | Bladder | + | + | + | + | + |
| | carcinoma | | | | | |

In order for cancer cells to be sensitive to the endoproteolytic cleavage, the target SNARE protein must be endogenously expressed and accessible to the light chain cleavage. To detect the presence of cleaved SNARE products a Western blot analysis was performed. Cells from the cell lines RT4, P19, NCl H69, NCl H82, DU145, T24, and J82, transfected and washed as described above, were lysed, by adding 200 μL of 2× SDS-PAGE Loading Buffer to each well, and the lysates were transferred to tubes and heated to 95° C. for 5 minutes. A 12 μL of each sample was separated by MOPS polyacrylamide gel electrophoresis using NuPAGE® Novex 4-12% Bis-Tris precast polyacrylamide gels (Invitrogen Inc., Carlsbad, Calif.) under denaturing, reducing conditions. Separated peptides were transferred from the gel onto nitrocellulose membranes by Western blotting using an electrophoretic tank transfer apparatus. The membranes were blocked by incubation, at room temperature, for 1 hour with gentle agitation, in a Blocking Solution containing Tris-Buffered Saline (TBS) (25 mM 2-amino-2-hydroxymethyl-1,3-propanediol hydrochloric acid (Tris-HCl)(pH 7.4), 137 mM sodium chloride, 2.7 mM potassium chloride), 0.1% polyoxyethylene (20) sorbitan monolaureate, 2% Bovine Serum Albumin (BSA), and 5% nonfat dry milk. Blocked membranes were incubated at 4° C. over night in TBS, 0.1% polyoxyethylene (20) sorbitan monolaureate, 2% BSA, and either 1) a 1:5,000 dilution of S9684 α-SNAP-25 rabbit polyclonal antiserum as the primary antibody (Sigma, St. Louis, Mo.); 2) a 1:5,000 dilution of sc17836 α-Syntaxin-1 rabbit polyclonal antiserum as the primary antibody (Santa Cruz Biotechnology, Santa Cruz, Calif.); or 3) a 1:5,000 dilution of sc69706 α-VAMP-2 mouse polyclonal antiserum as the primary antibody (Santa Cruz Biotechnology, Santa Cruz, Calif.). Primary antibody probed blots were washed three times for 5 minutes each time in TBS, polyoxyethylene (20) sorbitan monolaureate. Washed membranes were incubated at room temperature for 1 hour in TBS, 0.1% polyoxyethylene (20) sorbitan monolaureate, 2% BSA containing either 1) a 1:5,000 dilution of 81-6720 goat polyclonal α-mouse immunoglobulin G, heavy and light chains (IgG, H+L) antibody conjugated to horseradish peroxidase (Invitrogen, Inc., Carlsbad, Calif.) as a secondary antibody; or 2) a 1:5,000 dilution of 81-6120 goat polyclonal α-rabbit immunoglobulin G, heavy and light chains (IgG, H+L) antibody conjugated to horseradish peroxidase (Invitrogen, Inc., Carlsbad, Calif.) as a secondary antibody. Secondary antibody-probed blots were washed three times for 5 minutes each time in TBS, 0.1% polyoxyethylene (20) sorbitan monolaureate. Signal detection of the labeled SNARE products were visualized using the ECL Plus™ Western Blot Detection System, a chemiluminescence-based detection system, (GE Healthcare-Amersham, Piscataway, N.J.). The membranes were imaged and the percent of cleaved SNARE product were quantified with a Typhoon 9410 Variable Mode Imager and Imager Analysis software (GE Healthcare-Amersham, Piscataway, N.J.). The data shows that SNAP-25 and VAMP-2 were expressed in some cell types, while Syntaxin was expressed in each cell type tested (Table 7).

TABLE 7
Presence of SNARE in Cells

| | | SNARE Presence in Cells | | |
|---|---|---|---|---|
| Cell Line | Origin | SNAP-25 | VAMP-2 | Syntaxin-1 |
| RT4 | Bladder | − | + | + |
| | carcinoma | | | |
| P19 | Embryonic | + | − | + |
| | carcinoma | | | |
| NCI H69 | Small cell Lung | ND | ND | ND |
| | carcinoma | | | |
| NCI H82 | Small cell Lung | ND | ND | ND |
| | carcinoma | | | |
| DU145 | Prostate | + | + | + |
| | carcinoma | | | |
| T24 | Bladder | − | + | + |
| | carcinoma | | | |
| J82 | Bladder | + | − | + |
| | carcinoma | | | |

In addition, the data shows that 1) BoNT/A light chain was able to cleave SNAP-25 present in cells from a P19 embryonic carcinoma cell line, a DU145 prostate carcinoma cell line, and a J82 urinary bladder carcinoma cell line (Table 8); 2) BoNT/E light chain was able to cleave SNAP-25 present in cells from a P19 embryonic carcinoma cell line and a J82 urinary bladder carcinoma cell line (Table 8); 3) BoNT/B light chain was unable to cleave VAMP-2 in all cell lines tested (Table 8); and 4) BoNT/C1 light chain was able to cleave Syntaxin-1 present in cells from a T24 urinary bladder carcinoma cell line (Table 8). These results indicate that treatment of cancer cells with the appropriate Clostridial toxin light chain will cleave one of three SNARE proteins to inhibit exocytosis. This inhibition will prevent the release of growth factors, angiogenic factors, and anti-apoptotic survival factors necessary for cancer cell growth and survival.

TABLE 8
Cleavage of SNARE by Light Chain

| | | SNARE Cleavage by Light Chain | | | |
|---|---|---|---|---|---|
| | | SNAP-25 | | VAMP-2 | Syntaxin-1 |
| Cell Line | Origin | LC/A | LC/E | LC/B | LC/C1 |
| RT4 | Bladder | − | − | − | − |
| | carcinoma | | | | |
| P19 | Embryonic | + | + | − | − |
| | carcinoma | | | | |
| NCI H69 | Small Cell Lung | ND | ND | ND | ND |
| | carcinoma | | | | |
| NCI H82 | Small Cell Lung | ND | ND | ND | ND |
| | carcinoma | | | | |
| DU145 | Prostate | + | − | − | − |
| | carcinoma | | | | |
| T24 | Bladder | − | − | − | + |
| | carcinoma | | | | |
| J82 | Bladder | + | + | − | − |
| | carcinoma | | | | |

To further test whether SNARE cleavage disrupts exocytosis, an insulin release assay was performed. HIT-T15 cells release insulin when placed in high concentration of glucose. It has also been shown these cells express SNAP-25, and that SNAP-25 is an integral component of the SNARE complex needed for insulin release. HIT-T15 cells, transfected and washed as described above, were placed in DMEM media containing either 1) 5.6 mM glucose for basal insulin release (low glucose); or 2) 25.2 mM glucose for evoked insulin release (high glucose). Cells were incubated in a 37° C. incubator under 5% carbon dioxide for approximately 1 hour to allow for insulin release. The incubated media was collected and the amount of insulin released was determined using an insulin ELISA kit. The assay was performed according to the manufacturer's instructions (APLCO Diagnostics, Salem, N.H.). Exocytosis was expressed as the amount of insulin released per 1×10⁶ cells per hour.

The data shows that HIT-T15 cells transfected with GFP-LC/A, GFP-LC/B, and GFP-LC/E released less insulin than untransfected cells or cells transfected with GFP (Table 9). In addition, the basal insulin released in media containing a low glucose concentration (5.6 mM) remained unchanged between the transfected cells. The data indicate that BoNT/A, BoNT/B and BoNT/E light chains inhibited the release of insulin by cleaving SNAP-25 or VAMP-2 in HIT-T15 cells.

TABLE 9
Insulin Release from HIT-H15 Cells

| Construct | 5.6 mM Glucose (Low) | 25.2 mM Glucose (High) |
|---|---|---|
| Untransfected | 6.5 +/− 0.1 | 9.9 +/− 2.9 |
| Control | | |
| GFP | 4.3 +/− 0.7 | 10.8 +/− 2.1 |
| GFP-LCA | 3.2 +/− 0.4 | 4.5 +/− 0.6 |
| GFP-LCB | 3.4 +/− 0.2 | 5.5 +/− 0.9 |
| GFP-LCE | 4.2 +/− 0.7 | 4.4 +/− 1.0 |

The botulinum toxin light chain activity may also inhibit the trafficking of proteins to and from the plasma membrane. To test whether SNARE cleavage disrupts delivery and localization of receptors to the plasma membrane, the presence or absence of cell membrane proteins was determined in cells transfected with botulinum toxin light chains. Cells from the cell lines DU145 and J82, transfected and washed as described above, were treated with 2 mM NHS-LC-Biotin (Thermo Scientific, Rockford, Ill.) at 4° C. for 2 hours. The cells were then treated with 250 mM Tris-HCl (pH 7.5) for 30 minutes at 4° C., and then washed three times in TBS. Membranes proteins were isolated using the Membrane Protein extraction kit (Calbiochem, San Diego, Calif.) according to the manufacturer's instructions. The biotinylated proteins were precipitated with immobilized-avidin (Thermo Scientific, Rockford, Ill.). After three washes with TBS, the samples were suspended in 50 μL 2× SDS-PAGE loading buffer and separated by MOPS polyacrylamide gel electrophoresis using NuPAGE® Novex 4-12% Bis-Tris precast polyacrylamide gels (Invitrogen Inc., Carlsbad, Calif.) under denaturing, reducing conditions. The gel was washed and fixed in 10% methanol and 7% acetic acid for 30 minutes. The wash solution was removed and the gel incubated in SYPRO® Ruby protein gel stain solution (Bio-Rad Laboratories, Hercules, Calif.) for 3 hours to overnight at room temperature. The stained gel was destained in 10% methanol and 7% acetic acid for 30 minutes. Chemiluminescence from the destained gel was visualized with a Typhoon 9410 Variable Mode Imager and Imager Analysis software (GE Healthcare-Amersham, Piscataway, N.J.). The data show that treatment with a BoNT/A light chain inhibits the trafficking of proteins to and from the plasma membrane, which would necessarily affect the population of receptors located on the surface of the cell. This disrupted trafficking may cause the cancer cells to become more sensitive to apoptotic factors and less sensitive to growth signals and angiogenic factors.

By establishing the SNARE cleavage effects by the light chains, and which light chains cleaved which SNARE proteins in each cell line, TVEMPs were subsequently designed in a manner that targeted the TVEMP to receptors that were overexpressed or uniquely expressed in cancers cells in order to deliver the catalytic light chain.

### Example 2

### Presence of Receptor and Target in Cancer Cells

This example illustrates how to determine the presence of a cognate receptor that can bind with the targeting moiety of a TVEMP disclosed herein as well as the presence of the target SNARE protein of the enzymatic domain of a TVEMP disclosed herein.

In order for a TVEMP to be an effective agent for the methods of treating cancer disclosed herein, the cancer cells must express the appropriate receptor that can bind with the targeting moiety of a TVEMP as well as the appropriate SNARE protein that can be cleaved by the enzymatic domain of the TVEMP.

To culture cells, an appropriate density of cells were plated into the wells of 96-well tissue culture plates containing 100 μL of an appropriate medium (Table 10), but without serum, and with or without 25 μg/mL of GT1b (Alexis Biochemicals, San Diego, Calif.). Cells were plated and incubated in a 37° C. incubator under 5% carbon dioxide until the cells differentiated, as assessed by standard and routine morphological criteria, such as growth arrest (approximately 3 days). The media was aspirated from each well and replaced with 100 μL of fresh media containing various concentrations of the botulinum toxin or TVEMP being tested in order to generate a full dose-response. The assay was done in triplicate. After 24 hrs treatment, the cells were washed, incubated for an additional two days without toxin or TVEMP to allow for the cleavage of the SNARE substrate. After this incubation, the cells were washed by aspirating the media and rinsing each well with 3 mL of 1× PBS. The cells were harvested by lysing in freshly prepared Lysis Buffer (50 mM HEPES, 150 mM NaCl, 1.5 mM MgCl₂, 1 mM EGTA, 1% , 4-octylphenol polyethoxylate) at 4° C. for 30 minutes with constant agitation. Lysed cells were centrifuged at 4000 rpm for 20 min at 4° C. to eliminate debris using a bench-top centrifuge. The total protein concentrations of the cell lysates were measured by Bradford assay.

TABLE 10
Cell Lines and Media

| Cell Line | Origin | Source | Serum Growth Media Composition |
|---|---|---|---|
| RT4 | Human urinary | ATCC HTB-2 | McCoy's 5a media with 10% fetal bovine |
| | bladder transitional | | serum, 100 U/mL Penicillin, and 100 μg/mL |
| | cell carcinoma | | Streptomycin |
| P19 | Mouse embryonic | ATCC CRL-1825 | Alpha Minimal Essential Medium media |
| | carcinoma | | with 7.5% bovine calf serum, 2.5% fetal |
| | | | bovine calf serum, 100 U/mL Penicillin, and |
| | | | 100 μg/mL Streptomycin |
| NCI H69 | Human small lung | ATCC HTB-119 | RPMI-1640 media with 10% fetal bovine |
| | carcinoma | | serum, 100 U/mL Penicillin, and 100 μg/mL |
| | | | Streptomycin |
| NCI H82 | Human small lung | ATCC HTB-175 | RPMI-1640 media with 10% fetal bovine |
| | carcinoma | | serum, 100 U/mL Penicillin, and 100 μg/mL |
| | | | Streptomycin |
| DU-145 | Human prostate | ATCC HTB-81 | Eagle's Minimum Essential Medium with 10% |
| | carcinoma derived | | fetal bovine serum, 100 U/mL Penicillin, |
| | from brain | | and 100 μg/mL Streptomycin |
| PC-3 | Human prostate | ATCC CRL-1435 | F-12K media with 10% fetal bovine serum, |
| | carcinoma derived | | 100 U/mL Penicillin, and 100 μg/mL |
| | from brain | | Streptomycin |
| LNCaP clone | Human prostate | ATCC CRL-1740 | RPMI-1640 Eagle's with 10% fetal bovine |
| FGC | carcinoma derived | | serum, 100 U/mL Penicillin, and 100 μg/mL |
| | from brain | | Streptomycin |
| RWPE-1 | Human prostate | ATCC CRL-11609 | Dulbecco's Minimum Essential Medium with |
| | | | 10% Fetal Bovine Serum, 2 mM |
| | | | GlutaMAX ™ I with 0.1 mM Non-Essential |
| | | | Amino-Acids, 10 mM HEPES, 1 mM |
| | | | Sodium Pyruvate, 100 U/mL Penicillin, and |
| | | | 100 μg/mL Streptomycin |
| T24 | Human urinary | ATCC HTB-4 | McCoy's 5a media with 10% fetal bovine |
| | bladder transitional | | serum, 100 U/mL Penicillin, and 100 μg/mL |
| | cell carcinoma | | Streptomycin |
| J82 | Human urinary | ATCC HTB-1 | Eagle's Minimum Essential Medium with 10% |
| | bladder transitional | | fetal bovine serum, 100 U/mL Penicillin, |
| | cell carcinoma | | and 100 μg/mL Streptomycin |
| MCF-7 | Human breast | ATCC HTB-22 | Dulbecco's Minimum Essential Medium with |
| | carcinoma | | 10% Fetal Bovine Serum, 2 mM |
| | | | GlutaMAX ™ I with 0.1 mM Non-Essential |
| | | | Amino-Acids, 10 mM HEPES, 1 mM |
| | | | Sodium Pyruvate, 100 U/mL Penicillin, and |
| | | | 100 μg/mL Streptomycin |
| SiMa | Human | DSMZ ACC 164 | RPMI 1640 with 10% Fetal Bovine Serum, |
| | neuroblastoma | | 0.1 mM Non-Essential Amino-Acids, 10 mM |
| | | | HEPES, 1 mM Sodium Pyruvate, 100 U/mL |
| | | | Penicillin, and 100 μg/mL Streptomycin, |
| 266.6 | Mouse pancreatic | ATCC CRL-2151 | Dulbecco's Minimum Essential Medium with |
| | | | 10% Fetal Bovine Serum, 2 mM |
| | | | GlutaMAX ™ I with 0.1 mM Non-Essential |
| | | | Amino-Acids, 10 mM HEPES, 1 mM |
| | | | Sodium Pyruvate, 100 U/mL Penicillin, and |
| | | | 100 μg/mL Streptomycin |
| HIT-T15 | Hamster pancreatic | ATCC CRL-1777 | Eagle's Minimum Essential Medium (low |
| | islet of Langerhans | | glucose) with 10% fetal bovine serum, 100 |
| | beta cells | | U/mL Penicillin, and 100 μg/mL |
| | | | Streptomycin |
| HUVEC | Human Umbilical | Cell Applications, Inc., | Endothelial Cell Growth Medium (Cell |
| | Vein Endothelial | San Diego, CA, Cat. | Applications, Inc., San Diego, CA, Cat. No. |
| | Cells | No. 200-05n | 211-500) |

To determine whether a cancer cell expresses the appropriate receptor and target SNARE protein, a Western blot analysis can be performed.

In one experiment, cells from the cell lines RT4, P19, NCl H69, NCl H82, DU-145, T24, J82, LNCaP, and PC-3, transfected and washed as described above, were harvested by adding 40 μL of 2× SDS-PAGE Loading Buffer (Invitrogen, Inc., Carlsbad, Calif.) and heating the plate to 95° C. for 5 min. A 12 μL of the harvested sample was separated by MOPS polyacrylamide gel electrophoresis under denaturing, reducing conditions using 1) CRITERION® 12% Bis-Tris precast polyacrylamide gels (Bio-Rad Laboratories, Hercules, Calif.), when separating the SNAP-25₁₉₇ cleavage product; 2) NuPAGE® 12% Bis-Tris precast polyacrylamide gels (Invitrogen Inc., Carlsbad, Calif.), when separating both the uncleaved SNAP-25₂₀₆ substrate and the SNAP-25₁₉₇ cleavage product; or 3) NuPAGE® Novex 4-12% Bis-Tris precast polyacrylamide gels (Invitrogen Inc., Carlsbad, Calif.), when separating all other proteins. Separated peptides were transferred from the gel onto nitrocellulose membranes by Western blotting using a electrophoretic tank transfer apparatus. The membranes were blocked by incubation at room temperature for 1 hour with gentle agitation in a Blocking Solution containing Tris-Buffered Saline (TBS) (25 mM 2-amino-2-hydroxymethyl-1,3-propanediol hydrochloric acid (Tris-HCl)(pH 7.4), 137 mM sodium chloride, 2.7 mM potassium chloride), 0.1% polyoxyethylene (20) sorbitan monolaureate, 2% Bovine Serum Albumin (BSA), and 5% nonfat dry milk. Blocked membranes were incubated at 4° C. overnight in TBS, 0.1% polyoxyethylene (20) sorbitan monolaureate, 2% BSA, and either 1) a 1:5,000 dilution of S9684 α-SNAP-25 rabbit polyclonal antiserum as the primary antibody (Sigma, St. Louis, Mo.); 2) a 1:5,000 dilution of sc123 α-Syntaxin-1 rabbit polyclonal antiserum as the primary antibody (Santa Cruz Biotechnology, Santa Cruz, Calif.); 3) a 1:5,000 dilution of sc13992 α-VAMP-1/2/3 rabbit polyclonal antiserum as the primary antibody (Santa Cruz Biotechnology, Santa Cruz, Calif.); 4) a 1:5,000 dilution of sc50371 α-SNAP-23 rabbit polyclonal antiserum as the primary antibody (Santa Cruz Biotechnology, Santa Cruz, Calif.); 5) a 1:5,000 dilution of sc28955 α-SVC2 rabbit polyclonal antiserum as the primary antibody (Santa Cruz Biotechnology, Santa Cruz, Calif.); 6) a 1:5,000 dilution of sc123 α-FGFR3 rabbit polyclonal antiserum as the primary antibody (Santa Cruz Biotechnology, Santa Cruz, Calif.); 7) a 1:5,000 dilution of sc9112 α-KOR1 rabbit polyclonal antiserum as the primary antibody (Santa Cruz Biotechnology, Santa Cruz, Calif.); 8) a 1:5,000 dilution of H00004987-D01 P α-OPRL1 rabbit polyclonal antiserum as the primary antibody (Novus Biologicals, Littleton, Colo.); and 9) a 1:5,000 dilution of sc47778 α-β-actin mouse monoclonal antiserum as the primary antibody (Santa Cruz Biotechnology, Santa Cruz, Calif.). Primary antibody probed blots were washed three times for 5 minutes each time in TBS, polyoxyethylene (20) sorbitan monolaureate. Washed membranes were incubated at room temperature for 1 hour in TBS, 0.1% polyoxyethylene (20) sorbitan monolaureate, 2% BSA containing either 1) a 1:5,000 dilution of 81-6720 goat polyclonal α-mouse immunoglobulin G, heavy and light chains (IgG, H+L) antibody conjugated to horseradish peroxidase (Invitrogen, Inc., Carlsbad, Calif.) as a secondary antibody; or 2) a 1:5,000 dilution of 81-6120 goat polyclonal α-rabbit immunoglobulin G, heavy and light chains (IgG, H+L) antibody conjugated to horseradish peroxidase (Invitrogen, Inc., Carlsbad, Calif.) as a secondary antibody. Secondary antibody-probed blots were washed three times for 5 minutes each time in TBS, 0.1% polyoxyethylene (20) sorbitan monolaureate. Signal detection of the labeled SNARE products were visualized using the ECL Plus™ Western Blot Detection System, a chemiluminescence-based detection system (GE Healthcare-Amersham, Piscataway, N.J.). The membranes were imaged and the percent of cleaved SNARE product was quantified with a Typhoon 9410 Variable Mode Imager and Imager Analysis software (GE Healthcare-Amersham, Piscataway, N.J.). The data shows that this approach can identify the receptors and SNARE proteins present in the cells comprising each cell line (Table 11).

TABLE 11
Expression of Receptors and SNARE Proteins in Cells

| | Expression | | | | | | | |
|---|---|---|---|---|---|---|---|---|
| Cell Line | SNAP-25 | SNAP-23 | VAMP-2 | Syntaxin-1 | FGFR3 | SV2C | OPRL-1 | KOR-1 |
| RT4 | + | − | + | + | + | + | ND | + |
| P19 | + | − | − | + | + | − | ND | + |
| NCI H69 | + | − | + | + | + | − | ND | + |
| NCI H82 | + | − | + | + | + | − | ND | + |
| DU-145 | ++ | + | ++ | ++ | +++ | ND | ND | + |
| PC-3 | − | ++ | +/− | ++ | +++ | ND | ND | + |
| LNCaP | + | + | + | + | +++ | +++ | ++ | + |
| clone FGC | | | | | | | | |
| T24 | − | ++ | + | + | ++ | ++ | ++ | + |
| J82 | ++ | +/− | ++ | + | +++ | ++ | ++ | + |

ND, not determined

Once cell lines comprising cells including the appropriate receptor and SNARE proteins were identified, the ability of a botulinum toxin or TVEMP to intoxicate these cells can be determined by detecting the presence of cleaved SNARE products using Western blot analysis. An appropriate density of cells from each cell line to be tested are plated into the wells of 96-well tissue culture plates containing 100 μL of an appropriate medium (Table 7) with or without 25 μg/mL of GT1b (Alexis Biochemicals, San Diego, Calif.). Cells are plated and incubated in a 37° C. incubator under 5% carbon dioxide until the cells differentiated, as assessed by standard and routine morphological criteria, such as growth arrest (approximately 3 days). The media is aspirated from each well and is replaced with 100 μL of fresh media containing various concentrations of the botulinum toxin or TVEMP being tested sufficient to generate a full dose-response. The assay is done in triplicate. After 24 hrs treatment, the cells are washed, incubated for an additional two days without toxin or TVEMP to allow for the cleavage of the SNARE substrate. After this incubation, the cells are washed by aspirating the media and rinsing each well with 3 mL of 1× PBS. The cells are harvested by lysing in freshly prepared Lysis Buffer (50 mM HEPES, 150 mM NaCl, 1.5 mM MgCl₂, 1 mM EGTA, 1% , 4-octylphenol polyethoxylate) at 4° C. for 30 minutes with constant agitation. Lysed cells are centrifuged at 4000 rpm for 20 min at 4° C. to eliminate debris using a bench-top centrifuge. The protein concentrations of cell lysates are measured by Bradford assay. Samples of the cell lysates are analyzed by Western blot analysis as described above.

In one experiment, differentiated cells from the cell lines LNCaP, J82, and MCF-7, transfected as described above. The media was aspirated from each well and the differentiated cells were treated by replacing with fresh media containing either 1) 0 (untreated sample), 0.12 nM, 0.36 nM, 1.1 nM, 3.3 nM, 10 nM, 30 nM, and 90 nM of a BoNT/A; 2) 0 (untreated sample), and 50 nM of a BoNT/A; 3) 0 (untreated sample), 0.12 nM, 0.36 nM, 1.1 nM, 3.3 nM, 10 nM, 30 nM, and 90 nM of a TVEMP designated Noci-LH_{N}/A; or 4) 0 (untreated sample), and 166 nM of a TVEMP designated Noci-LHN/A. After 1) 3-15 hours; 2) 6 hours or 3) 24 hours treatment, the cells were washed, incubated for an additional 16 hours without toxin or TVEMP to allow for the cleavage of the SNAP-25 substrate. After this incubation, the cells were washed and harvested as described above. The presence of cleaved SNAP-25 product was detected using Western blot analysis as described above using a 1:5,000 dilution of S9684 α-SNAP-25 rabbit polyclonal antiserum as the primary antibody (Sigma, St. Louis, Mo.) as the primary antibody and a 1:5,000 dilution of 81-6120 goat polyclonal α-rabbit immunoglobulin G, heavy and light chains (IgG, H+L) antibody conjugated to horseradish peroxidase (Invitrogen, Inc., Carlsbad, Calif.) as a secondary antibody. These results are shown in Table 12.

TABLE 12
Cleavage of SNARE Substrate

Lowest Concentration and Earliest
Time for Cleavage Detection

| Cell Line | BoNT/A | Noci-LH_{N}/A |
|---|---|---|
| LNCaP | 50 nM at 9 hours | 166 nM at 9 hours |
| J82 | 50 nM at 3 hours | 166 nM at 3 hours |
| | 1.1 nM at 24 hours | |
| MCF-7 | 1.1 nM at 6 hours | ND |

ND, not determined

Taken together, the data shows that 1) BoNT/A was able to cleave SNAP-25 present in cells from a LNCaP prostate carcinoma cell line, a J82 urinary bladder carcinoma cell line, and a MCF-7 breast carcinoma cell line (Table 9); 2) Noci-LH_{N}/A was able to cleave SNAP-25 present in cells from a LNCaP prostate carcinoma cell line and a J82 urinary bladder carcinoma cell line (Table 9). These results indicate that treatment of cancer cells with the appropriate Clostridial toxin light chain will cleave one of three SNARE proteins to inhibit exocytosis. This inhibition will prevent the release of growth factors, angiogenic factors, and anti-apoptotic survival factors necessary for cancer cell growth and survival. Lastly, these experiments illustrate the validity of the general concept that intracellular delivery of a botulinum light chain into cancer cells results in cleavage of the appropriate SNARE protein not only by transfecting light chain constructs, but also by using the endogenous signal transduction pathway for the targeting domain.

### Example 3

### Effects of Light Chain Delivery on Angiogenesis

This example illustrates that treatment with a botulinum toxin or TVEMP will affect angiogenesis to a degree sufficient to provide a therapeutic benefit in a cancer treatment.

The blockade of exocytosis resulting from a treatment with botulinum toxin or TVEMP based on LHN/A-G will likely prevent the release of angiogenic factors, including, e.g., Vascular endothelial growth factor (VEGF), Fibroblast Growth Factor-1 (FGF1) and FGF2. Preventing the release of these angiogenic factors will reduce, or altogether inhibit, angiogenesis in the area where the toxin or TVEMP is administered. To test whether such a treatment reduces or inhibits angiogenesis, four different assays were performed: a VEGF release assay, a cell migration assay, an in vitro blood vessel formation assay, and a human angiogenesis protein array assay.

VEGF is known to be a potent mitogen for vascular endothelial cells and an inducer of physiological and pathological angiogenesis. To validate the potential for a botulinum toxin or TVEMP in inhibiting angiogenesis, the ability of a toxin or TVEMP to inhibit release of VEGF from a cell was assessed. To conduct a VEGF release assay, about 600,000 cells from a SiMa cell line were plated into the wells of 6-well collagen IV tissue culture plates containing 3 mL of a serum-free medium containing Minimum Essential Medium, 2 mM GlutaMAX™ I with Earle's salts, 1×B27 supplement, 1×N2 supplement, 0.1 mM Non-Essential Amino Acids, 10 mM HEPES and 25 μg/mL GT1b. These cells were incubated in a 37° C. incubator under 5% carbon dioxide until the cells differentiated, as assessed by standard and routine morphological criteria, such as growth arrest and neurite extension (approximately 3 days). The media from the differentiated cells was aspirated from each well and replaced with fresh media containing either 0.77 mg/mL of a BoNT/A or 1 mg/mL of a Noci-LH_{N}/A TVEMP. As a control, cells were treated with media alone in parallel. After treatment the media was removed and replaced with fresh differentiation media. A 60 μL aliquot of media was removed from each well and replaced with 100 μL differentiation media 1 day, 2 days, 3 days, and 4 days after the addition of fresh differentiation media. The removed media was stored at −20° C. until needed. After the last sample was removed, the cells were trypsinized and the number of cells in each well was counted.

The presence of VEGF in the collected samples was detected using a K151BMB-1 VEGF tissue culture assay (Meso Scale Discovery, Gaithersburg, Md.). A MULTI-ARRAY® 96-well Small Spot Plate VEGF plate was blocked with 150 μL Blocking Buffer (PBS with 0.05% polyoxyethylene (20) sorbitan monolaureate, 2% ECL Blocking reagent (GE Healthcare-Amersham, Piscataway, N.J.), and 1% goat serum (Rockland Immunochemicals, Gilbertsville, Pa.) and shaken at 600 rpm for one hour. The blocking buffer was discharged and 25 μL of each sample was added to each well of the VEGF plate and the plate was incubated at 4° C. for 2 hours. The plate was washed three times with 200 μL PBS-T (PBS plus 0.05% Tween-20) and then 25 μl of SULFO-TAG α-hVEGF mouse monoclonal antibody 5 μg/mL in 2% antibody buffer (PBS plus 0.05% polyoxyethylene (20) sorbitan monolaureate, and 2% ECL Blocking reagent (GE Healthcare-Amersham, Piscataway, N.J.) added and incubated on a shaker at 600 rpm at RT for 1 hour. Plates were washed three times with PBS-T and then 150 μL Read Buffer (MSD, Cat #R92TC-1) were added per well. Plates were read in a SECTOR™ Imager 6000 Image Reader (Meso Scale Discovery, Gaithersburg, Md.). The data was then exported into Microsoft Office Excel 2007. The amount of VEGF detected was normalized to the number of cells present in the well and the percent VEGF release value was calculated using the control as the 100% value.

The data shows that treatment with BoNT/A inhibits VEGF release by about 50% in SiMa cells (Table 13). Although the addition of Noci-LH_{N}/A TVEMP did not appear to inhibit VEGF release, this result could be due to the lower potency of Noci-LH_{N}/A TVEMP compared to BoNT/A in SiMa cells. The EC₅₀ of BoNT/A in differentiated SiMa cells is less than about 0.5 nM, while the EC₅₀ of Noci-LH_{N}/A TVEMP is more than 30 nM. As such, the lack of effect of Noci-LH_{N}/A TVEMP in SiMa cells is simply due to the low amount of OPRL-1 receptor present in these cells. This lack of effect corroborates the concept that cells expressing low levels of the targeted receptor will not be affected by botulinum toxin or TVEMP treatment (i.e. normal cells surrounding tumors over-expressing a receptor of interest). In addition, the finding that the addition of IL-6, a known transcriptional regulator of VEGF, had no effect on VEGF release is consistent with reports that the addition of exogenous IL-6 does not affect VEGF secretion.

TABLE 13
VEGF Release Assay

| Time | VEGF Release | | |
|---|---|---|---|
| Point | Control | BoNT/A | Noci-LH_{N}/A TVEMP |
| Day 1 | 100% | 69% | 119% |
| Day 2 | 100% | 57% | 123% |
| Day 3 | 100% | 53% | 125% |
| Day 4 | 100% | 57% | 104% |

Since VEGF is an inducer of migration, a compound that affects the release of VEGF should effect migration as well. Moreover, inhibition of exocytosis by a compound will also inhibit the release of additional factors involved in cell migration. To determine whether a botulinum toxin or TVEMP treatment could reduce or inhibit cell migration, a cell migration assay (Essen Bioscience, Ann Arbor, Mich.) was performed according to the manufacturer's instructions. On day 1, DU-145 cells were plated at 25,000 cells per well in a 96-well Essen ImageLock plate in growth media. On day 2 the cells were treated with either 10 nM BoNT/A, 40 nM Noci-LH_{N}/A TVEMP, or 90 nM Gal-LH_{N}/A TVEMP in growth media. As a positive control for inhibition of migration, cells were treated with 0.11 μM, 0.33 μM, or 1 μM Cytochalasin-D. As a negative control, cells were treated with media alone. On day 3, after the cells had reached 100% confluence, the cells were washed with media and then a 96-pin WoundMaker (Essen Bioscience, Ann Arbor, Mich.) was used to simultaneously create wounds in all the wells. After cell wounding, the media was removed and the cells were washed two times with 150 μL Dulbecco's Phosphate Buffered Saline with Ca²⁺ and Mg²⁺ and then 100 μL of media was added. The plate was then placed in an INCUCYTE™ scanner (Essen Bioscience, Ann Arbor, Mich.) and images were taken every 1 hour for 45 consecutive hours. The data was analyzed as relative wound density versus time using the INCUCYTE™ Cell Migration software. Relative wound density is designed to be zero at time zero, and 100% when the cell density inside the wound is the same as the cell density outside the initial wound.

The results are presented in Table 14. The results showed that cells pre-treated with either Noci-LH_{N}/A TVEMP or Gal-LH_{N}/A TVEMP migrated slightly slower than cells treated with media alone. The result showed that treatment with Noci-LH_{N}/A TVEMP or Gal-LH_{N}/A TVEMP resulted in a significant reduction in cell migration after 24 hours, about 10% reduction when compared to cells treated with media alone. Cells treated with BoNT/A did not exhibit an affect on cell migration. The cells treated with Cytochalasin-D did not migrate. When the same experiment was performed with PC-3 cells, that do not contain SNAP-25, rather than a reduction, an increase in migration was observed (data not shown), suggesting that initially, likely via activation of their ligand receptors, BoNT/A, Noci-LH_{N}/A TVEMP, and Gal-LH_{N}/A TVEMP function to increase migration. But after cleavage of SNAP-25 migration is reduced. As such, a longer exposure to a botulinum toxin and/or TVEMP will most likely result in more dramatic reduction in migration of such treated cells.

TABLE 14
Cell Migration Assay

Relative Wound Density at 24 Hours

| | | Percent Relative |
|---|---|---|
| Treatment | Mean | to Media |
| Media Control | 78.2 ± 2.4 | 100% |
| BoNT/A | 78.6 ± 1.1 | 101% |
| Noci-LH_{N}/A TVEMP | 71.5 ± 3.3 | 91% |
| Gal-LH_{N}/A TVEMP | 69.5 ± 4.4 | 89% |
| Cytochalasin-D | 3.3 ± 0.2 | 4% |

Angiogenesis involves multiple steps; to achieve new blood vessel formation, endothelial cells must first escape their stable location by breaking through the basement membrane. Once this is achieved, endothelial cells migrate towards an angiogenic stimulus that might be released from cancer cells, or wound-associated macrophages. In addition, endothelial cells proliferate to provide the necessary number of cells for making a new vessel. Subsequent to this proliferation, the new outgrowth of endothelial cells needs to reorganize into a three-dimensionally tubular structure. To determine whether a botulinum toxin or TVEMP treatment could reduce or inhibit blood vessel formation, an in vitro Endothelial Tube Formation assay (Cell Biolabs, Inc., San Diego, Calif.) was performed according to the manufacturer's instructions. Human Umbilical Vein Endothelial Cells (HUVECs) were grown to 80% confluence in T-75 culture flasks until confluent. Cells were harvested and then plated at 500,000 cells per well for HUVECs in a 6-well plate for 24 hours. After incubation, cells were either kept untreated or treated with 2 nM or 5 nM of BoNT/A or 6 nM or 25 nM of Noci-LH_{N}/A TVEMP for 24 hours. As a positive control for inhibition, cells were treated with a collagenase inhibitor. As a negative control for inhibition, cells were treated with media alone. The cells were then harvested again and plated at 35,000 cells per well onto the ECM gel prepared from murine Engelbreth-Holm-Swan (EHS) tumor cells, which contain multiple angiogenic stimulating factors, such as, e.g., laminin, type IV collagen, heparan sulfate proteoglycans, entactin and growth factors such as FGF2 and TGF-βs. The cells were incubated for 3-4 hours on the ECM gels and then inspected under a microscope and photographed, either before or after staining with Calcein AM.

A Endothelial Tube Formation assay was also modified to use cells from a tumor cell line. In this modified assay, cells from a LNCaP, PC-3, DU-145, T24, and J82 cell lines were grown to 80% confluence in T-75 culture flasks. Cells were then harvested and plated at 400,000 cell per well in a 6-well plate containing 3 mL of an appropriate medium (Table 10), but with 1% serum. Cells were incubated in a 37° C. incubator under 5% carbon dioxide for 3 days. After incubation, cells were either kept untreated or treated with 20 nM of BoNT/A or 40 nM of Noci-LH_{N}/A TVEMP for 24 hours. The cells were then harvested, plated on ECM gel plates and inspected as described above.

The results show that in HUVEC, DU145 and J82 cells, and to a lesser degree in T24 and LNCaP cells, tubes formed on ECM plates treated with media alone, whereas treatment with a collagenase inhibitor prevented the formation of tubes (Table 15). No tubes formed in PC-3 cells. BoNT/A and Noci-LH_{N}/A TVEMP treatment of cells from a LNCaP prostate carcinoma cell line and a J82 bladder carcinoma cell line inhibited the formation of tubes. BoNT/A and Noci-LH_{N}/A TVEMP treatment had no effect on tube formation from HUVEC cultures. This inhibition of tube formation maybe due to inhibition of migration, delivery of receptors and other proteins to the membrane (motility factors and their receptors), adhesion molecules that interact with the matrix or other cells, and/or secretion of proteases.

TABLE 15
Endothelial Tube Formation Assay

| | Inhibition of Endothelial Tube Formation | | | |
|---|---|---|---|---|
| Cell | | Collagenase | | |
| Line | Media | Inhibitor | BoNT/A | Noci-LH_{N}/A |
| LNCaP | No | Yes | Yes | Yes |
| PC-3 | — | — | — | — |
| DU-145 | No | ND | ND | ND |
| T24 | No | ND | ND | ND |
| J82 | No | Yes | Yes | Yes |
| HUVEC | No | ND | No | No |

ND, not determined

To conduct a human angiogenesis protein array screen, cells from a DU-145 prostate cancer cell line were plated in a 100 mm² plate containing Eagle's Minimum Essential Medium with 1% charcoal stripped FBS, 100 U/mL Penicillin, and 100 μg/mL Streptomycin. Cells were grown to a density of 5×10⁶ cells by incubating in a 37° C. incubator under 5% carbon dioxide overnight. After this incubation, the cells were washed by aspirating the media and rinsing the plate with 10 mL of 1×PBS. The washed cells were treated by replacing with fresh media containing 50 nM BoNT/A. For comparison, cells treated with media alone were run in parallel. After 24 hour treatment, the cells were washed, and harvested by lysing in freshly prepared Lysis Buffer (50 mM HEPES, 150 mM NaCl, 1.5 mM MgCl₂, 1 mM EGTA, 1% , 4-octylphenol polyethoxylate) on ice for 30 minutes with constant gentle agitation. Lysed cells were centrifuged at 14,000 g for 5 minutes at 4° C. to eliminate debris. The protein concentrations of cell lysates were measured by Bradford assay. To perform an assay, an array was incubated with 250 μL of each cell lysate containing 500 pg of protein. Array images were captured by scanning the blots with a Typhoon 9410 Imager and quantitation of array was performed with Image Quant TL V2005. Fold increased was determined by dividing signal from untreated over treated sample.

The results show that the majority of the 35 angiogenesis-related proteins detected were up-regulated in the cells treated with BoNT/A, compared to the untreated control (Table 16). Proteins that increased in expression were involved in promoting angiogenesis except for two proteins that are anti-angiogenic (endostatin and angiostatin). There was increased presence of GDNF, PDGF-AA, and FGF1 that promote cell proliferation, differentiation, cell growth and development. Proteins that promote or initiate angiogenesis were; Coagulation Factor III, EG-VEGF, Angiopoetin-1, Angiopoetin-2, and PD-ECGF. Expressions in proteins involved in glucose metabolism were; DPPIV, IGFBP-1, IGFBP-2, and IGFBP-3. Proteins that enhance cell-cell adhesion were also up-regulated; MIP-1, MMP-9, Endothelin-1, Platelet Factor 4 and TGF-β1. The most significant increase was observed for Endocrine gland-derived vascular endothelial growth factor (EG-VEGF), which was almost 100-fold increased. The increase of these proteins in cell lysates may reflect their accumulation in the cytoplasm since exocytosis has been inhibited and the cells cannot release them to the media.

TABLE 16
Human Angiogenesis Array in DU145 Cell line

| | Mean Pixels Density | | Fold | |
|---|---|---|---|---|
| Analyte | Untreated | Treated | Increased | Function |
| External Control | 65451 | 68877 | 1.1 | — |
| Internal Control | 50052 | 59543 | 1.2 | — |
| Coagulation Factor III/TF | 12736 | 26726 | 2.1 | Promotes angiogenesis |
| GDNF | 156 | 428 | 2.7 | Promotes survival and differentiation |
| MIP-1 alpha | 153 | 535 | 3.5 | Chemotaxis |
| CXCL 16 | 3465 | 2352 | 0.7 | Cytokine |
| GM-CSF | 5001 | 1457 | 0.3 | Cytokine |
| Serpin E1 | 677 | 2214 | 3.3 | Inhibit proteases |
| Activin A | 552 | 1672 | 3.0 | Regulate morphogenesis in prostate |
| DPPIV | 3790 | 8923 | 2.4 | Glucose metabolism |
| HB-EGF | 8990 | 6717 | 0.7 | Cell proliferation |
| MMP-9 | 2454 | 5050 | 2.1 | Breakdown extracellular matrix |
| Serpin F1 | 743 | 882 | 1.2 | Inhibit proteases |
| TIMP-1 | 95918 | 86280 | 0.9 | Anti-angiogenic |
| Angiogenin | 6022 | 5468 | 0.9 | Promotes angiogenesis |
| EG-VEGF | 15 | 1368 | 88.3 | Promotes angiogenesis |
| IGFBP-1 | 122 | 1147 | 9.4 | Insulin growth factor protein |
| Pentraxin 3 | 119 | 732 | 6.2 | Involved in complement-mediated |
| | | | | clearance of apoptotic cells |
| TIMP-4 | 152 | 845 | 5.6 | Matrix metalloproteinases inhibitor |
| Angiopoietin-1 | 137 | 807 | 5.9 | Promotes angiogenesis |
| IGFBP-2 | 2379 | 8330 | 3.5 | Insulin growth factor protein |
| PD-ECGF | 942 | 12924 | 13.7 | Promotes angiogenesis |
| Thrombospondin-1 | 2138 | 12359 | 5.8 | Anti-angiogenic |
| Angiopoietin-2 | 129 | 1985 | 15.3 | Antagonist of angiopoietin 1 |
| Endostatin/Collagen XVIII | 2388 | 6800 | 2.8 | Anti-angiogenic |
| IGFBP-3 | 1145 | 11329 | 9.9 | Insulin like promotes cell survivor |
| PDGF-AA | 202 | 908 | 4.5 | Regulates cell proliferation, cellular |
| | | | | differentiation, cell growth, development |
| Angiostatin/Plasminogen | 142 | 893 | 6.3 | Anti-angiogenic |
| Endothelin-1 | 581 | 5828 | 10.0 | Vascular homeostasis |
| uPA | 30656 | 57108 | 1.9 | Serine protease |
| Amphiregulin | 33908 | 20736 | 0.6 | Interacts with the EGF/TGF-alpha |
| | | | | receptor to promote the growth |
| FGF1 | 1189 | 1875 | 1.6 | Promotes proliferation & differentiation |
| IL-8 | 45837 | 19261 | 0.4 | Angiogenic factor |
| FGF2 | 28018 | 23513 | 0.8 | Promotes proliferation & differentiation |
| LAP/TGF-β1 | 360 | 1914 | 5.3 | Increases extracellular matrix |
| | | | | production |
| Platelet Factor 4 | 456 | 819 | 1.8 | Cytokine |
| VEGF | 33513 | 31434 | 0.9 | Affects permeability |

Taken together, the experiments described in this Example show an overall decrease in angiogenic potential after treatment with botulinum toxin of TVEMP together with an observed increase in intracellular angiogenic proteins. This could be due to either activation of receptors for botulinum toxin or TVEMP that promotes angiogenesis and/or accumulation of vesicular proteins due to blockage of exocytosis after cleavage of SNARE proteins.

### Example 4

### Effects of Light Chain Delivery on Apoptosis

This example illustrates that treatment with a botulinum toxin or TVEMP will affect apoptosis to a degree sufficient to provide a therapeutic benefit in a cancer treatment.

The blockade of exocytosis resulting from a treatment with botulinum toxin or TVEMP based on LHN/A-G will likely result in decreased metabolic activity and decreased cell viability. As such, cancer cells with inhibited exocytosis capability due to a toxin or TVEMP effect will have a reduced ability to survive. To test whether such a treatment causes decreased cancer cell viability, three different assays were performed: a cell viability and metabolism assay, a Caspase-3/8 activity assay, and a human apoptotic protein array assay.

To determine whether a botulinum toxin or TVEMP treatment could decrease cancer cell viability, a CELLTITER 96® AQueous One Solution Cell Proliferation Assay cell metabolic activity assay (Promega Corp., Madison, Wis.) was performed according to the manufacturer's instructions. This assay is a colorimetric assay containing a tetrazolium compound [3-(4,5-dimethylthiazol-2-yl)-5-(3-carboxymethoxyphenyl)-2-(4-sulfophenyl)-2H-tetrazolium, inner salt; MTS] that is reduced by NADPH or NADH in metabolically active cells. The reduced MTS is a colored formazan product that can be measured at an absorbance of 490 nm. An appropriate density of cells from the cell lines MCF-7, SiMa, PC-12, 266.6, RWPE-1, and N2a, were plated into the wells of 96-well tissue culture plates containing 100 μL of an appropriate medium (Table 7), but without serum, and with or without 25 μg/mL of GT1b (Alexis Biochemicals, San Diego, Calif.). Cells were plated and incubated in a 37° C. incubator under 5% carbon dioxide until the cells differentiated, as assessed by standard and routine morphological criteria, such as growth arrest (approximately 3 days). The media was aspirated from each well and the differentiated cells were treated by replacing with fresh media containing 0 (untreated sample), 0.3125 nM, 1.25 nM, and 20 nM of a BoNT/A. After 24 hrs treatment, the cells were washed by aspirating the media and rinsing each well with 100 μL of 1× PBS. After washing, 100 μL of MTS solution was added to each well, incubated for 2 hours, and then the absorbance at 490 nm recorded with a 96-well plate reader. The quantity of formazan product as measured by the amount of 490 nm absorbance is directly proportional to the number of living cells in culture. A similar design can be employed to examine the effects of a TVEMP on cell viability.

The results show that a BoNT/A treatment decreased the metabolic activity in the cancerous cell lines tested (Table 17).

TABLE 17
Cell Metabolic Activity Assay

BoNT/A Concentration

| Cell Line | 0 nM | 0.3125 nM | 1.25 nM | 20 nM |
|---|---|---|---|---|
| MCF-7 | 1.60 | 1.45 | 1.41 | 1.30 |
| SiMa | 1.68 | 1.40 | 1.07 | 0.33 |
| PC-12 | 1.68 | 1.66 | 1.45 | 1.15 |
| 266.6 | 1.10 | 1.05 | 1.02 | 0.82 |
| RWPE-1 | 0.99 | 1.01 | 0.89 | 0.67 |
| N2a | 1.63 | 1.50 | 1.43 | 1.28 |

To further demonstrate that a botulinum toxin or TVEMP treatment could decrease cancer cell viability, a CELLTITER GLO® Luminescent Cell Viability Assay (Promega Corp., Madison, Wis.) was performed according to the manufacturer's instructions. In this assay, cell viability is quantified on the bases of the presence of ATP, which signals the presence of metabolically active cells. A decreased in ATP content corresponds to less metabolically active cells. Cells from the cell lines LNCaP, J82, T24, and DU-145 were differentiated as described above. The media was aspirated from each well and the differentiated cells were treated by replacing with fresh media containing either 1) 0 (untreated sample), 25 nM, and 50 nM of a BoNT/A; or 2) 0 (untreated sample), 250 nM, and 500 nM of a Noci-LH_{N}/A TVEMP. After 24 hrs treatment, the cells were washed by aspirating the media and rinsing each well with 100 μL of 1× PBS. After washing, 100 μL of CELLTITER GLO® reagent was added to each well. After ten minutes incubation at room temperature, the sample luminescence was measured using a SpectraMAX L luminescence reader (Molecular Devices, Sunnyvale, Calif.). Assays were performed in triplicate and cell viability was noted every day for four or five days.

The data shows that decreased viability was observed in cells from both a DU-145 prostate carcinoma cell line and a J82 bladder carcinoma cell line after BoNT/A treatments (Table 18) or Noci-LH_{N}/A TVEMP treatments (Table 19).

TABLE 18
Cell Viability Assay for BoNT/A

| | BoNT/A Concentration | | | | | | | |
|---|---|---|---|---|---|---|---|---|
| | DU-145 | | | | J82 | | | |
| Time | 0 nM | 25 nM | 0 nM | 50 nM | 0 nM | 25 nM | 0 nM | 50 nM |
| Day 1 | 3356 | 3291 | 404219 | 301228 | 3077 | 2853 | 543436 | 318900 |
| | | (0.385) | | (0.325) | | (0.223) | | (0.398) |
| Day 2 | 2360 | 2433 | 649139 | 394645 | 5211 | 4646 | 741025 | 493817 |
| | | (0.433) | | (0.174) | | (0.016) | | (0.129) |
| Day 4 | ND | ND | 1277552 | 809182 | ND | ND | 1242627 | 649797 |
| | | | | (0.058) | | | | (0.010) |
| Day 5 | 4823 | 2325 | ND | ND | 7384 | 4262 | ND | ND |
| | | (0.0001) | | | | (0.0001) | | |

P value indicating significant difference relative to non-treated control is listed in parenthesis.
ND, not determined

TABLE 19
Cell Viability Assay for Noci-LH_{N}/A TVEMP

| | Noci-LH_{N}/A TVEMP Concentration | | | | | | | |
|---|---|---|---|---|---|---|---|---|
| | DU-145 | | | | J82 | | | |
| Time | 0 nM | 250 nM | 0 nM | 500 nM | 0 nM | 250 nM | 0 nM | 500 nM |
| Day 1 | 3356 | 3630 | 404219 | 408023 | 3077 | 3189 | 543436 | 406420 |
| | | (0.087) | | (0.959) | | (0.223) | | (0.103) |
| Day 2 | 2360 | 2379 | 649139 | 622596 | 5211 | 4639 | 741025 | 677236 |
| | | (0.876) | | (0.802) | | (0.015) | | (0.581) |
| Day 4 | | | 1277552 | 1030346 | | | 1242627 | 854124 |
| | | | | (0.171) | | | | (0.020) |
| Day 5 | 4823 | 3595 | | | 7384 | 6349 | | |
| | | (0.0003) | | | | (0.009) | | |

P value indicating significant difference relative to non-treated control is listed in parenthesis.
ND, not determined

To determine whether a botulinum toxin or TVEMP treatment decreased cancer cell viability by an apoptotic process, the activity of Caspase-3/8 was measured in cell treated with BoNT/A. Cells from the cell lines LNCaP, J82, and T24 were differentiated as described above. The media was aspirated from each well and the differentiated cells were treated by replacing with fresh media containing either 1) 0 (untreated sample), 0.5 nM, 5 nM, and 50 nM of a BoNT/A; or 2) 0 (untreated sample), 1.6 nM, 16 nM, and 166 nM of a Noci-LH_{N}/A TVEMP. After 24 hrs treatment, the cells were washed by aspirating the media and rinsing each well with 100 μL of 1× PBS To measure cellular caspase 9 activity, 50 μL of CASPASE-GLO® 9 (Promega, Corp., Madison, Wis.) reagent was added to the culture media of each well. After 30 minute incubation at 37° C., the luminescence of each sample was measured using a Spectramax L luminometer (Molecular Devices, Sunnyvale, Calif.). T24 does not express SNAP-25 and should not be sensitive to treatment with BoNT/A or Noci-LH_{N}/A TVEMP.

The data shows that an effect on Caspase 3/8 activity was most prevalent in LNCaP cell after exposure to BoNT/A, indicating that LNCaP cell line viability decreases with BoNT/A treatment (Table 20). These data are supported by the cell viability assays measuring the number of live and dead cells in populations treated with BoNT/A (Table 18). Although cells from a J82 cell line did not show significant differences in Caspase 3/8 activity, this cell line did contain a higher amount of dead cells after BoNT/A or Noci-LH_{N}/A TVEMP treatments (Table 19). The reason for the observation of no caspase activity in J82 cells could be due to at least two possibilities: 1) the timing of BoNT/A treatment to detect Caspase 3/8 activity is different for J82 and LNCaP (e.g., Caspase 3/8 activation may had occur earlier in J82 cells); or 2) the cell death pathway for J82 is independent of Caspase 3/8.

TABLE 20
Caspase 3/8 Activity Assay

| | BoNT/A Concentration | | | | Noci-LH_{N}/A TVEMP | | | |
|---|---|---|---|---|---|---|---|---|
| Cell Line | 0 nM | 0.5 nM | 5 nM | 50 nM | 0 nM | 1.6 nM | 16 nM | 166 nM |
| LNCaP | 270 | 283 | 239 | 572 | 218 | 232 | 233 | 263 |
| T24 | 656 | 612 | 634 | 646 | 637 | 602 | 623 | 617 |
| J82 | 235 | 146 | 256 | 194 | 132 | 133 | 103 | 98 |

To test whether cell death of cells treated with a botulinum toxin or TVEMP was directed by a process independent of Caspase 3/8 pathway, cells were assayed for the presence of cleaved nuclear poly (ADP-ribose) polymerase (PARP). PARP is a 116 kDa nuclear poly (ADP-ribose) polymerase and appears to be involved in DNA repair in response to environmental stress. This protein can be cleaved by many ICE-like caspases in vitro and is one of the main cleavage targets of Caspase-3 in vivo. In human PARP, the cleavage occurs between Asp214 and Gly215, which separates the PARP amino-terminal DNA binding domain (24 kDa) from the carboxy-terminal catalytic domain (89 kDa). PARP helps cells to maintain their viability; cleavage of PARP facilitates cellular disassembly and serves as a marker of cells undergoing apoptosis. To determine whether changes in cell viability are due to cells undergoing apoptosis, cells from the cell lines DU-145 and J82 were differentiated as described above. The media was aspirated from each well and the differentiated cells were treated by replacing with fresh media containing either 1) 0 (untreated sample) and 50 nM of a BoNT/A; or 2) 0 (untreated sample) and 500 nM of a Noci-LH_{N}/A TVEMP. After 48 hrs treatment, the cells were washed, harvested and Western blot analysis performed as described in Example 1, except an α-PARP antibodies were used as the primary antibody. Cells from both cell lines showed an increased of cleaved PARP after 2 days of Noci-LH_{N}/A TVEMP treatment. However, the presence of cleaved PARP was minimal in cells from both cell lines treated with a BoNT/A.

To conduct a human apoptosis protein array screen, cells from a DU-145 prostate cancer cell line were treated with a BoNT/A, harvested, and assayed as described above in Example 3. The results show that after treatment of cells from the DU-145 cell line with 50 nM BonT/A for 24 hours, most of apoptosis-related proteins remained unchanged when compared to control. There were only 10 apoptotic-related proteins where expression decreased from 1.5-fold to 2.4-fold (Table 21). A decreased in expression was noted in three anti-apoptotic proteins (Livin, survivin, and BCL-x), two cell cycle related proteins (Claspin and P27), antioxidant related protein (PON2), chaperone protein (clusterin) and two pro-apoptotic related proteins (Bax and Cytochrome C).

TABLE 21
Human Apoptosis Array in DU-145 Cell line

| | Mean Pixel density | | Fold | |
|---|---|---|---|---|
| Analyte | Untreated | Treated | Decrease | Function |
| Livin | 644.1 | 469.7 | 1.7 | Anti-apoptotic |
| Cytochrome c | 3423 | 1889 | 1.9 | Pro-apoptotic |
| XIAP | 10099 | 10045 | 1.0 | Anti-apoptotic |
| HTRA2/Omi | 7542 | 9368 | 0.8 | IAP antagonist |
| Clusterin | 1139 | 816 | 1.6 | Chaperones misfolded proteins |
| TNF rRI/TNFRSF1A | 2036 | 1467 | 1.5 | Activates NFkB |
| HSP70 | 7058 | 9669 | 0.7 | Stress response chaperone |
| Claspin | 6630 | 3390 | 2.0 | Cell cycle check point |
| Survivin | 8717 | 3739 | 2.4 | Anti-apoptotic |
| HSP60 | 945 | 855 | 1.2 | Stress response chaperone |
| cIAP-2 | 2862 | 3156 | 0.9 | Inhibitor of Apoptosis (IAP) |
| SMAC/Diablo | 8379 | 7132 | 1.2 | Promotes caspase activation by interaction |
| | | | | with IAP proteins |
| HSP27 | 5716 | 5683 | 1.0 | Stress response chaperone |
| cIAP-1 | 16916 | 15297 | 1.1 | Inhibitor of Apoptosis (IAP) |
| Phospho-Rad17 | 1646 | 999 | 1.8 | cell cycle check point |
| HO-2/HMOX2 | 8930 | 8934 | 1.0 | Microsomal enzyme |
| Catalase | 18742 | 18710 | 1.0 | Prevent cell damage from oxidative stress |
| p53 | 19134 | 22007 | 0.9 | Induces apoptosis |
| HO-1/HMOX1/HSP32 | 9878 | 11333 | 0.9 | Microsomal enzyme |
| Cleaved Caspase-3 | 715 | 614 | 1.3 | Downstream mediator of apoptotis |
| p53 | 8623 | 11225 | 0.8 | Induces apoptosis |
| HIF-1 alpha | 6832 | 6703 | 1.0 | Binds to hypoxia response elements |
| Pro-Caspase-3 | 36318 | 42668 | 0.9 | Downstream mediator of apoptotis |
| p53 | 20019 | 24725 | 0.8 | Induces apoptosis |
| Fas/TNFSF6 | 34978 | 35878 | 1.0 | Induces apoptosis |
| Bcl-x | 571 | 445 | 1.6 | Anti-apoptotic |
| p27 | 1293 | 852 | 1.7 | Cell cycle check point |
| FADD | 9996 | 8647 | 1.2 | Induces apoptosis |
| Bcl-2 | 967 | 1427 | 0.7 | Anti-apoptotic |
| p21 | 1062 | 1029 | 1.1 | Blocks cell cycle |
| TRAIL R2/DR5 | 25985 | 21477 | 1.2 | Induces apoptosis |
| Bax | 2097 | 1436 | 1.6 | Apoptotic activator |
| PON2 | 2611 | 1784 | 1.5 | Antioxidant enzyme |
| TRAIL R1 | 28443 | 20518 | 1.4 | Induces apoptosis |
| Bad | 5097 | 5932 | 0.9 | Pro-apoptotic |

Taken together, the experiments described in this Example show that treatment with a BoNT/A or TVEMP results in decreased metabolic activity and decreased cells viability. Events related to apoptosis were identified following light chain delivery into cancer cells, Caspase 3/8 activity was observed after treatment with BoNT/A in LNCaP cells as well as increased cleavage of PARP, the main substrate for Caspase 3 was observed after treatment with Noci-LH_{N}/A TVEMP in the DU-145 and J82 cells, showing that cells are pushed towards apoptosis after treatment with a BoNT/A or a TVEMP. Overall, the amounts of proteins involved with apoptosis in the cell lysates did not change after treatment with BoNT/A. Most of the pro-apoptotic and anti-apoptotic proteins exert their function by translocating from the cytoplasm to the mitochondria without changes in total protein amount. The small changes detected may be a short term response of the tumor cells to the inhibition of exocytosis and the interference with the input from the autocrine or paracrine loops that the cancer cell needs to survive. Eventually these cells will be pushed into apoptosis due to the lack of survival signals.

### Example 5

### Construction of a TVEMP Comprising a PSMA Targeting Domain

The following example illustrates how to make a TVEMP comprising a PSMA targeting domain.

A polynucleotide molecule encoding TVEMP-PSMA/LHnA, a TVEMP comprising a PSMA targeting domain, a BoNT/A translocation domain, and a BoNT/A enzymatic domain, is synthesized using standard procedures (BlueHeron Biotechnology, Bothell, Wash.). TVEMP-PSMA/LHnA is a BoNT/A modified by replacing amino acids 874-1296 of SEQ ID NO: 1 or amino acids 1092-1296 of SEQ ID NO: 1 with a PSMA targeting domain comprising SEQ ID NO: 82. Oligonucleotides of 20 to 50 bases in length are synthesized using standard phosphoramidite synthesis. These oligonucleotides are hybridized into double stranded duplexes that are ligated together to assemble the full-length polynucleotide molecule. This polynucleotide molecule is cloned using standard molecular biology methods into a pUCBHB1 vector at the Smal site to generate pUCBHB1/TVEMP-PSMA/LHnA. The synthesized polynucleotide molecule is verified by sequencing using BIG DYE™ Terminator Chemistry 3.1 (Applied Biosystems, Foster City, Calif.) and an ABI 3100 sequencer (Applied Biosystems, Foster City, Calif.).

If desired, an expression optimized polynucleotide molecule encoding TVEMP-PSMA/LHnA disclosed above can be synthesized in order to improve expression in an Escherichia coli strain. The polynucleotide molecule encoding the TVEMP can be modified to 1) contain synonymous codons typically present in native polynucleotide molecules of an Escherichia coli strain; 2) contain a G+C content that more closely matches the average G+C content of native polynucleotide molecules found in an Escherichia coli strain; 3) reduce polymononucleotide regions found within the polynucleotide molecule; and/or 4) eliminate internal regulatory or structural sites found within the polynucleotide molecule, see, e.g., Lance E. Steward et al. Optimizing Expression of Active Botulinum Toxin Type E, WO 2005/020578; Lance E. Steward et al. Optimizing Expression of Active Botulinum Toxin Type A, WO 2005/027917. Once sequence optimization is complete, oligonucleotides of 20 to 50 bases in length are synthesized using standard phosphoramidite synthesis. These oligonucleotides are hybridized into double stranded duplexes that are ligated together to assemble the full-length polynucleotide molecule. This polynucleotide molecule is cloned using standard molecular biology methods into a pUCBHB1 vector at the SmaI site to generate pUCBHB1/TVEMP-PSMA/LHnA. The synthesized polynucleotide molecule is verified by sequencing using BIG DYE™ Terminator Chemistry 3.1 (Applied Biosystems, Foster City, Calif.) and an ABI 3100 sequencer (Applied Biosystems, Foster City, Calif.). Is so desired, optimization to a different organism, such as, e.g., a yeast strain, an insect cell-line or a mammalian cell line, can be done, see, e.g., Steward, supra, WO 2005/020578 and Steward, supra, WO 2005/027917.

A similar cloning strategy is used to make pUCBHB1 cloning constructs for TVEMP-PSMA/LHnB, a modified BoNT/B where amino acids 861-1291 or amino acids 1079-1291 of SEQ ID NO: 6 are replaced with a PSMA targeting domain comprising SEQ ID NO: 82; TVEMP-PSMA/LHnC1, a modified BoNT/C1 where amino acids 869-1291 or amino acids 1093-1291 of SEQ ID NO: 11 are replaced with a PSMA targeting domain comprising SEQ ID NO: 82; TVEMP-PSMA/LHnD, a modified BoNT/D where amino acids 865-1276 or amino acids 1080-1276 of SEQ ID NO: 13 are replaced with a PSMA targeting domain comprising SEQ ID NO: 82; TVEMP-PSMA/LHnE, a modified BoNT/E where amino acids 848-1252 or amino acids 1067-1252 of SEQ ID NO: 15 are replaced with a PSMA targeting domain comprising SEQ ID NO: 82; TVEMP-PSMA/LHnF, a modified BoNT/F where amino acids 866-1274 or amino acids 1087-1274 of SEQ ID NO: 18 are replaced with a PSMA targeting domain comprising SEQ ID NO: 82; TVEMP-PSMA/LHnG, a modified BoNT/G where amino acids 866-1297 or amino acids 1087-1297 of SEQ ID NO: 21 are replaced with a PSMA targeting domain comprising SEQ ID NO: 82; TVEMP-PSMA/LHnT, a modified TeNT where amino acids 882-1315 or amino acids 1109-1315 of SEQ ID NO: 22 are replaced with a PSMA targeting domain comprising SEQ ID NO: 82; TVEMP-PSMA/LHnBa, a modified BaNT where amino acids 858-1268 or amino acids 1076-1268 of SEQ ID NO: 23 are replaced with a PSMA targeting domain comprising SEQ ID NO: 82; and TVEMP-PSMA/LHnBu, a modified BuNT where amino acids 848-1251 or amino acids 1067-1251 of SEQ ID NO: 24 are replaced with a PSMA targeting domain comprising SEQ ID NO: 82. Similarly, the 13-trefoil domain from a Clostridial toxin indicated above can be replaced with a PSMA targeting domain comprising SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112.

To construct pET29/TVEMP-PSMA/LHnA, a pUCBHB1/TVEMP-PSMA/LHnA construct is digested with restriction endonucleases that 1) excise the insert comprising the open reading frame encoding TVEMP-PSMA/LHnA; and 2) enable this insert to be operably-linked to a pET29 vector (EMD Biosciences-Novagen, Madison, Wis.). This insert is subcloned using a T4 DNA ligase procedure into a pET29 vector that is digested with appropriate restriction endonucleases to yield pET29/TVEMP-PSMA/LHnA. The ligation mixture is transformed into chemically competent E. coli DH5a cells (Invitrogen, Inc, Carlsbad, Calif.) using a heat shock method, plated on 1.5% Luria-Bertani agar plates (pH 7.0) containing 50 μg/mL of Kanamycin, and placed in a 37° C. incubator for overnight growth. Bacteria containing expression constructs are identified as Kanamycin resistant colonies. Candidate constructs are isolated using an alkaline lysis plasmid mini-preparation procedure and analyzed by restriction endonuclease digest mapping to determine the presence and orientation of the insert. This cloning strategy yielded a pET29 expression construct comprising the polynucleotide molecule encoding TVEMP-PSMA/LHnA operably-linked to a carboxyl terminal polyhistidine affinity binding peptide.

A similar cloning strategy is used to make pET29 expression constructs comprising a polynucleotide molecule encoding for TVEMP-PSMA/LHnB, TVEMP-PSMA/LHnC1, TVEMP-PSMA/LHnD, TVEMP-PSMA/LHnE, TVEMP-PSMA/LHnF, TVEMP-PSMA/LHnG, TVEMP-PSMA/LHnT, TVEMP-PSMA/LHnBa, and TVEMP-PSMA/LHnBu, as well as the TVEMPs indicated above comprising a PSMA targeting domain including SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112.

### Example 6

### Treatment of Cancer

A physician examines a person who complains of stomach pain and diagnoses her with gastric cancer. The person is treated by local administration a composition comprising a TVEMP as disclosed herein in the vicinity of the affected area. The patient's condition is monitored and after about 1-7 days after treatment, the physician notes that the growth of the malignant tumor has slowed down. At one and three month check-ups, the physician determines that the size of the tumor has become smaller and there is a decrease amount of blood vessels associated with the tumor. This reduction in tumor size indicates successful treatment with the composition comprising a TVEMP. In addition, a systemic administration of a composition comprising a TVEMP as disclosed herein could also be used to administer a disclosed TVEMP to treat the gastric cancer.

A physician examines a person who complains of difficulty in urinating and diagnoses him with prostate cancer. The person is treated systemically by intravenous administration a composition comprising a TVEMP as disclosed herein. The patient's condition is monitored and after about 1-7 days after treatment, the physician determines that the size of the prostate has become smaller. At one and three month check-ups, the physician determines that the size of the prostate has returned to its normal size and that serum PSA levels are within the normal range. This reduction in tumor size and/or reduces serum PSA levels indicates successful treatment with the composition comprising a TVEMP. In addition, a local administration of a composition comprising a TVEMP as disclosed herein could also be used to administer a disclosed TVEMP to treat the prostate cancer.

A physician examines a person who complains of alternating boats of constipation and diarrhea as well as abdominal pain and diagnoses him with colon cancer. The person is treated systemically by intravenous administration a composition comprising a TVEMP as disclosed herein. The patient's condition is monitored and after about 1-7 days after treatment, the physician notes that the growth of the malignant tumor has slowed down. At one and three month check-ups, the person indicates that his bowel movements have returned to normal and the physician determines that the size of the tumor has become smaller and there is a decrease amount of blood vessels associated with the tumor. The normal bowel movements and/or the reduction in tumor size indicate successful treatment with the composition comprising a TVEMP. In addition, systemic administration could also be used to administer a disclosed TVEMP to treat cancer. In addition, administration by inhalation could also be used to administer a disclosed TVEMP to treat the colon cancer.

A physician examines a person who complains of pelvic pain and diagnoses her with rectal cancer. The person is treated by local administration a composition comprising a TVEMP as disclosed herein in the vicinity of the affected area. The patient's condition is monitored and after about 1-7 days after treatment, the physician notes that the growth of the malignant tumor has slowed down. At one and three month check-ups, the person indicates that the pelvic pain has subsided and the physician determines that the size of the tumor has become smaller and there is a decrease amount of blood vessels associated with the tumor. The reduced pain and/or the reduction in tumor size indicate successful treatment with the composition comprising a TVEMP. In addition, a systemic administration of a composition comprising a TVEMP as disclosed herein could also be used to administer a disclosed TVEMP to treat the rectal cancer.

A physician examines a person who complains of abdominal pain and diagnoses her with colon cancer. The person is treated by systemically by intravenous administration of a composition comprising a TVEMP as disclosed herein. The patient's condition is monitored and after about 1-7 days after treatment, and the physician notes that the growth of the malignant tumor has slowed down. At one and three month check-ups, the person indicates that the abdominal pain has subsided and the physician determines that the size of the tumor has become smaller and there is a decrease amount of blood vessels associated with the tumor. The reduced pain and/or the reduction in tumor size indicate successful treatment with the composition comprising a TVEMP. In addition, a local administration of a composition comprising a TVEMP as disclosed herein could also be used to administer a disclosed TVEMP to treat the colon cancer.

### Example 7

### Treatment of a Disease of Hyperproliferation

A physician examines a person who complains of difficulty in urinating and diagnoses him with BPH. The person is treated locally by intraglandular administration a composition comprising a TVEMP as disclosed herein into the prostate. The patient's condition is monitored and after about 7-14 days after treatment, the physician determines that the size of the prostate has become smaller. At one and three month check-ups, the physician determines that the size of the prostate has returned to its normal size. This reduction in prostate size indicates successful treatment with the composition comprising a TVEMP.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of excludes any element, step, or ingredient not specified in the claims. The transition term “consisting essentially of limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating a prostate cancer in a mammal, the method comprising the step of administering to the mammal in need thereof a therapeutically effective amount of a composition including a TVEMP comprising a Prostate-Specific Membrane Antigen targeting domain, a Clostridial toxin translocation domain and a Clostridial toxin enzymatic domain, and an exogenous protease cleavage site, wherein administration of the composition reduces a symptom associated with the prostate cancer.

2. The method of claim 1, wherein the TVEMP comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.

3. The method of claim 1, wherein the Prostate-Specific Membrane Antigen targeting domain comprises SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112.

4. The method of claim 1, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.

5. The method of claim 1, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.

6. The method of claim 1, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.

7. The method of claim 1, wherein the prostate cancer is a prostatic epithelial cancer, a prostatic intraepithelial neoplasia, or a prostatic adenocarcinoma.

8. A method of treating neovascularization or pathological angiogenesis associated with a cancer in a mammal, the method comprising the step of administering to the mammal in need thereof a therapeutically effective amount of a composition including a TVEMP comprising a Prostate-Specific Membrane Antigen targeting domain, a Clostridial toxin translocation domain and a Clostridial toxin enzymatic domain, and an exogenous protease cleavage site, wherein administration of the composition reduces a symptom associated with the neovascularization or pathological angiogenesis associated with a cancer.

9. The method of claim 8, wherein the TVEMP comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.

10. The method of claim 8, wherein the Prostate-Specific Membrane Antigen targeting domain comprises SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 112.

11. The method of claim 8, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.

12. The method of claim 8, wherein the neovascularization or pathological angiogenesis associated with a cancer is a gastric cancer or a colorectal cancer.

13. The method of claim 12, wherein the gastric cancer is a gastric adenocarcinoma, a gastric carcinoma, or a metastatic cancer originating from a gastric adenocarcinoma or a gastric carcinoma.

14. The method of claim 12, wherein the colorectal cancer is a colorectal adenocarcinoma, a colorectal carcinoma, or a metastatic cancer originating from a colorectal adenocarcinoma or a colorectal carcinoma.


SEQUENCE LISTING (122 sequences)

SEQ ID NO: 1 - protein, 1296 aa, Clostridium botulinum A1
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1296 aa, Clostridium botulinum A2
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 3 - protein, 1292 aa, Clostridium botulinum A3
MPFVNKPFNYRDPGNGVDIAYIKIPNAGQMQPVKAFKIHEGVWVIPERDTFTNPEEGDLNPPPEAKQVPVSYYDSTYLSTDNEKDNYLKGVIKLFDRIYSTGLGRMLLSFIVKGIPFWGGSTIDTELKVIDTNCINVIEPGGSYRSEELNLVITGPSADIIQFECKSFGHDVFNLTRNGYGSTQYIRFSPDFTFGFEESLEVDTNPLLGAGTFATDPAVTLAHELIHAAHRLYGIAINPNRVLKVKTNAYYEMSGLEVSFEELRTFGGNDTNFIDSLWQKKFSRDAYDNLQNIARILNEAKTIVGTTTPLQYMKNIFIRKYFLSEDASGKISVNKAAFKEFYRVLTRGFTELEFVNPFKVINRKTYLNFDKAVFRINIVPDENYTINEGFNLEGANSNGQNTEINSRNFTRLKNFTGLFEFYKLLCVRGIIPFKTKSLDEGYNKALNYLCIKVNNWDLFFSPSEDNFTNDLDKVEEITADTNIEAAEENISSDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAVIFLSWAEELVYDFTDETNEVTTMDKIADITIIVPYIGPALNIGNMVSKGEFVEAILFTGVVALLEFIPEYSLPVFGTFAIVSYIANKVLTVQTINNALSKRNEKWDEVYKYTVTNWLAKVNTQIDLIREKMKKALENQAEATRAIINYQYNQYTEEEKNNINFNIDDLSSKLNRSINRAMININKFLDQCSVSYLMNSMIPYAVKRLKDFDASVRDVLLKYIYDNRGTLILQVDRLKDEVNNTLSADIPFQLSKYVNDKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWMFVTITNNRLTKSKIYINGRLIDQKPISNLGNIHASNKIMFKLDGCRDPRRYIMIKYFNLFDKELNEKEIKDLYDSQSNPGILKDFWGNYLQYDKPYYMLNLFDPNKYVDVNNIGIRGYMYLKGPRGSVMTTNIYLNSTLYMGTKFIIKKYASGNEDNIVRNNDRVYINVVVKNKEYRLATNASQAGVEKILSALEIPDVGNLSQVVVMKSKDDQGIRNKCKMNLQDNNGNDIGFVGFHLYDNIAKLVASNWYNRQVGKASRTFGCSWEFIPVDDGWGESSL

SEQ ID NO: 4 - protein, 1296 aa, Clostridium botulinum A4
MPLVNQQINYYDPVNGVDIAYIKIPNAGKMQPVKAFKIHNKVWVIPERDIFTNPEEVDLNPPPEAKQVPISYYDSAYLSTDNEKDNYLKGVIKLFERIYSTDLGRMLLISIVRGIPFWGGGKIDTELKVIDTNCINIIQLDDSYRSEELNLAIIGPSANIIESQCSSFRDDVLNLTRNGYGSTQYIRFSPDFTVGFEESLEVDTNPLLGAGKFAQDPAVALAHELIHAEHRLYGIAINTNRVFKVNTNAYYEMAGLEVSLEELITFGGNDAKFIDSLQKKEFSLYYYNKFKDIASTLNKAKSIVGTTASLQYMKNVFKEKYLLSEDATGKFLVDRLKFDELYKLLTEIYTEDNFVKFFKVLNRKTYLNFDKAVFKINIVPDVNYTIHDGFNLRNTNLAANFNGQNIEINNKNFDKLKNFTGLFEFYKLLCVRGIITSKTKSLDEGYNKALNELCIKVNNWDLFFSPSEDNFTNDLDKVEEITSDTNIEAAEENISLDLIQQYYLNFNFDNEPENTSIENLSSDIIGQLEPMPNIERFPNGKKYELNKYTMFHYLRAQEFKHSNSRIILTNSAKEALLKPNIVYTFFSSKYIKAINKAVEAVTFVNWIENLVYDFTDETNEVSTMDKIADITIVIPYIGPALNIGNMIYKGEFVEAIIFSGAVILLEIVPEIALPVLGTFALVSYVSNKVLTVQTIDNALSKRNEKWDEVYKYIVTNWLAIVNTQINLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVNRLKDKVNNTLSADIPFQLSKYVDNKKLLSTFTEYIKNITNASILSIVYKDDDLIDLSRYGAEIYNGDKVYYNSIDKNQIRLINLESSTIEVILKKAIVYNSMYENFSTSFWIRIPKYFNSISLNNEYTIINCMENNSGWKVSLNYGEIIWTFQDTQEIKQRVVFKYSQMINISDYINRWIFVTITNNRITKSKIYINGRLIDQKPISNLGNIHASNKIMFKLDGCRDPHRYIVIKYFNLFDKELSEKEIKDLYDNQSNSGILKDFWGDYLQYDKSYYMLNLYDPNKYVDVNNVGIRGYMYLKGPRDNVMTTNIYLNSSLYMGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWRERPL

SEQ ID NO: 5 - protein, 1296 aa, Clostridium botulinum A5
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTELGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGEHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPEVNYTIYDGFNLRNTNLAANFNGQNTEINNMNFTKLKNFTGLFEFYKLLCVRGIITSKTKSLDEGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIVLTNSVNEALLNPSSVYTFFSSDYVRKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWGEVYKYIVTNWLAKVNTQIDLIRKKMKEALENQAEATKAIINYQYNQYTEEEKNNINFNIGDLSSKLNDSINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASEINIGSKVNFDPIDKNQIQLFNLESSKIEIILKNAIVYNSMYENFSTSFWIKIPKYFSKINLNNEYTIINCIENNSGWKVSLNYGEIIWTLQDNKQNIQRVVFKYSQMVAISDYINRWIFITITNNRLNNSKIYINGRLIDQKPISNLGNIHASNNIMFKLDGCRDPQRYIWIKYFNLFDKELNEKEIKDLYDNQSNSGILKDFWGNYLQYDKPYYMLNLYDPNKYVDVNNVGIRGYMYLKGPRGSIVTTNIYLNSSLYMGTKFIIKKYASGNKDNIVRNNDRVYINVVVKNKEYRLATNASQAGVEKILSVLEIPDVGNLSQVVVMKSKNDQGIRNKCKMNLQDNNGNDIGFIGFHQFNNIDKLVASNWYNRQIERSSRTFGCSWEFIPVDDGWGESPL

SEQ ID NO: 6 - protein, 1291 aa, Clostridium botulinum B1
MSVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPAYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYIYKYFKKEEEKLFLAPISDSDEFYNTIQIKEYDEQPTYSCQLLFKKDEESTDEIGLIGIHRFYESGIVFKEYKDYFCISKWYLKEVKRKPYNSKLGCNWQFIPKDEGWTE

SEQ ID NO: 7 - protein, 1291 aa, Clostridium botulinum B2
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSKKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSKYTNNTILIEIFNKYNSEILNNIILNLRYRDNNLIDLSGYGANVEVYDGVELNDKNQFKLTSSTNSEIRVTQNQNIIFNSMFLDFSVSFWIRIPKYKNDGIQNYIHNEYTIINCIKNNSGWKISIRGNRIIWTLTDINGKTKSVFFEYSIRKDVSEYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSSVGEILTRSKYNQNSNYINYRNLYIGEKFIIRRKSNSQSINDDIVRKEDYIYLDFFNSNREWRVYAYKDFKEEEKKLFLANIYDSNEFYKTIQIKEYDEQPTYSCQLLFKKDEESTDEIGLIGIHRFYESGTVFKNYKDYFCISKWYLKEVKRKPYNSDLGCNWKFIPKDEGWTE

SEQ ID NO: 8 - protein, 1291 aa, Clostridium botulinum B3
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPRIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSMYTNNTILIEIFNKYNSEILNNIILNLRYRDNNLIDLSGYGAKVEVYNGVELNDKNQFKLTSSANSKIRVTQNQDIIFNSMFLDFSVSFWIRIPKYKNDGIQNYIHNEYTIINCIKNNSGWKISIRGNKIIWTLTDINGKTKSVFFEYSIRKDVSEYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSSVGEILTRSKYNQNSNYINYRNLYIGEKFIIRRKSNSQSINDDIVRKEDYIYLDFFNLNQEWRVYAYKDFKKKEEKLFLANIYDSNEFYNTIQIKEYDEQPTYSCQLLFKKDEESTDEIGLIGIHRFYESGIVFKDYKDYFCISKWYLKEVKRKPYNPNLGCNWQFIPKDEGWIE

SEQ ID NO: 9 - protein, 1291 aa, Clostridium botulinum Bnp
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVEQKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDTIQAEELYTFGGQDPSIISPSTDKSIYDKVLQNFRGIVDRLNKVLVCISDPNININIYKNKFKDKYKFVEDSEGKYSIDVESFNKLYKSLMFGFTEINIAENYKIKTRASYFSDSLPPVKIKNLLDNEIYTIEEGFNISDKNMGKEYRGQNKAINKQAYEEISKEHLAVYKIQMCKSVKVPGICIDVDNENLFFIADKNSFSDDLSKNERVEYNTQNNYIGNDFPINELILDTDLISKIELPSENTESLTDFNVDVPVYEKQPAIKKVFTDENTIFQYLYSQTFPLNIRDISLTSSFDDALLVSSKVYSFFSMDYIKTANKVVEAGLFAGWVKQIVDDFVIEANKSSTMDKIADISLIVPYIGLALNVGDETAKGNFESAFEIAGSSILLEFIPELLIPVVGVFLLESYIDNKNKIIKTIDNALTKRVEKWIDMYGLIVAQWLSTVNTQFYTIKEGMYKALNYQAQALEEIIKYKYNIYSEEEKSNININFNDINSKLNDGINQAMDNINDFINECSVSYLMKKMIPLAVKKLLDFDNTLKKNLLNYIDENKLYLIGSVEDEKSKVDKYLKTIIPFDLSTYTNNEILIKIFNKYNSEILNNIILNLRYRDNNLIDLSGYGAKVEVYDGVKLNDKNQFKLTSSADSKIRVTQNQNIIFNSMFLDFSVSFWIRIPKYRNDDIQNYIHNEYTIINCMKNNSGWKISIRGNRIIWTLIDINGKTKSVFFEYNIREDISEYINRWFFVTITNNLDNAKIYINGTLESNMDIKDIGEVIVNGEITFKLDGDVDRTQFIWMKYFSIFNTQLNQSNIKEIYKIQSYSEYLKDFWGNPLMYNKEYYMFNAGNKNSYIKLVKDSSVGEILIRSKYNQNSNYINYRNLYIGEKFIIRRKSNSQSINDDIVRKEDYIHLDFVNSNEEWRVYAYKNFKEQEQKLFLSIIYDSNEFYKTIQIKEYDEQPTYSCQLLFKKDEESTDDIGLIGIHRFYESGVLRKKYKDYFCISKWYLKEVKRKPYKSNLGCNWQFIPKDEGWTE

SEQ ID NO: 10 - protein, 1291 aa, Clostridium botulinum Bbv
MPVTINNFNYNDPIDNNNIIMMEPPFARGMGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVNDLPIVPNEKKFFMQSTDAIQAEELYTFGGQDPSIISPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVKAPGICIDVDNEDLFFIADKNSFSDDLSKNERIAYNTQNNYIENDFSINELILDTDLISKIELPSENTESLTDFNVYVPVYKKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIETINSALTKRDEKWIDMYGLIVAQWLSTVNTQFYTIKEGMYKALNYQAQALEEIIKYKYNIYSEKERSNINIDFNDVNSKLNEGINQAIDNINNFINECSVSYLMKKMIPLAVEKLLDFDNTLRKNLLNYIDENKLYLIGSAEYEKSKVDKYLKTSIPFDLSTYTNNTILIEIFNKYNSDILNNIILNLRYRDNKLIDLSGYGAKVEVYDGVKLNDKNQFKLTSSANSKIRVIQNQNIIFNSMFLDFSVSFWIRIPKYKNDGIQNYIHNEYTIINCMKNNSGWKISIRGNMIIWTLIDINGKIKSVFFEYSIKEDISEYINRWFFVTITNNSDNAKIYINGKLESHIDIRDIREVIANDEIIFKLDGNIDRTQFIWMKYFSIFNTELSQSNIEEIYKIQSYSEYLKDFWGNPLMYNKEYYMFNAGNKNSYIKLKKDSSVGEILTRSKYNQNSKYINYRDLYIGEKFIIRRKSNSQSINDDIVRKEDYIYLDFFNLNQEWRVYMYKYFKKEEEKLFLAPISDSDEFYNTIQIKEYDEQPTYSCQLLFKKDEESTDEIGLIGIHRFYESGIVFKEYKDYFCISKWYLKEVKRKPYNSKLGCNWQFIPKDEGWTE

SEQ ID NO: 11 - protein, 1291 aa, Clostridium botulinum C1-1
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 12 - protein, 1280 aa, Clostridium botulinum C1-2
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHTMHNLYGIAIPNDQRISSVTSNIFYSQYKVKLEYAEIYAFGGPTIDLIPKSGRKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINVETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRENFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKVNESFENTIPFNIFSYTNNSLLKDIINEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLNEVKLDKTIVFGIDENIDENQMLWIRDFNIFSKELSNEDINIVYEGQILRNVIKDYWGNPLKFDTEYYIINDNYIDRYIAPKSNILVLVQYPDRSKLYTGNPITIKSVSDKNPYSRILNGDNIMFHMLYNSGKYMIIRDTDTIYAIEGRECSKNCVYALKLQSNLGNYGIGIFSIKNIVSQNKYCSQIFSSFMKNTMLLADIYKPWRFSFENAYTPVAVTNYETKLLSTSSFWKFISRDPGWVE

SEQ ID NO: 13 - protein, 1276 aa, Clostridium botulinum D1
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 14 - protein, 1285 aa, Clostridium botulinum D2
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTRFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIRGNTNDTRVRGENVLYFNTTIDNKQYSLGMYKPSRNLGTDLVPLGALDQPMDEIRKYGSFIIQPCNTFDYYASQLFLSSNATTNRLGILSIGSYSFKLGDDYWFNHEYLIPVIKIEHYASLLESTSTHWVFVPASE

SEQ ID NO: 15 - protein, 1252 aa, Clostridium botulinum E1
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 16 - protein, 1252 aa, Clostridium botulinum E2
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGIQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYNNEPNANILKDFWGNYLLYDKEYYLLNVLKPNNFIDRRTDSTLSINNIRSTILLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 17 - protein, 1252 aa, Clostridium botulinum E3
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHELIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 18 - protein, 1274 aa, Clostridium botulinum F1
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 19 - protein, 1280 aa, Clostridium botulinum F2
MPVVINSFNYNDPVNDETILYMQKPYEERSRKYYKAFEIMPNVWIMPERDTIGTKPDEFQVPDSLKNGSSAYYDPNYLTTDAEKDRYLKTMIKLFNRINSNPTGKVLLEEVSNARPYLGDDDTLINEFLPVNVTTSVNIKFSTDVESSIISNLLVLGAGPDIFKAYCTPLVRFNKSDKLIEPSNHGFGSINILTFSPEYEHIFNDISGGNHNSTESFIADPAISLAHELIHALHGLYGAKAVTHKESLVAERGPLMIAEKPIRLEEFLTFGGEDLNIIPSAMKEKIYNDLLANYEKIATRLREVNTAPPGYDINEYKDYFQWKYGLDRNADGSYTVNRNKFNEIYKKLYSFTEIDLANKFKVKCRNTYFIKYGFVKVPNLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEESKVYTFFSSEFIDTINKPVNAALFIDWISKVIRDFTTEATQKSTVDKIADISLIVPYVGLALNIVIEAEKGNFEEAFELLGAGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESKYNINNIEEELNKKVSLAMKNIERFMTESSISYLMKLINEAEVGKLKEYDKHVKSDLLDYILYHKLILGEQTKELIDLVTSTLNSSIPFELSSYTNDKILIIYFNRLYKKIKDSSILDMRYENNKFIDISGYGSNISINGNVYIYSTNRNQFGIYSGRLSEVNIAQNNDIIYNSRYQNFSISFWVTIPKHYRPMNRNREYTIINCMGNNNSGWKISLRTIRDCEIIWTLQDTSGNKEKLIFRYEELASISDYINKWIFVTITNNRLGNSRIYINGNLIVEKSISNLGDIHVSDNILFKIVGCDDETYVGIRYFKVFNTELDKTEIETLYSNEPDPSILKDYWGNYLLYNKKYYLFNLLRKDKYITRNSGILNINQQRGVTGGISVFLNYKLYEGVEVIIRKNAPIDISNTDNFVRKNDLAYINVVDHGVEYRLYADISITKSEKIIKLIRTSNPNDSLGQIIVMDSIGNNCTMNFQNNDGSNIGLLGFHSDDLVASSWYYNHIRRNTSSNGCFWSFISKEHGWKE

SEQ ID NO: 20 - protein, 1278 aa, Clostridium botulinum F3
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 21 - protein, 1297 aa, Clostridium botulinum G
  VARIANT 7: Identity of amino acid is unknown
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 22 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 23 - protein, 1268 aa, Clostridium baratii
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESNGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYVILLSNYTAIASRLSQVNRNNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLIVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLRAEYNIYSIKEELNKKVSLAMQNIDRFLTESSISYLMKLINEAKINKLSEYDKRVNQYLLNYILENSSTLGTSSVPELNNLVSNTLNNSIPFELSEYTNDKILIHILIRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 24 - protein, 1251 aa, Clostridium butyricum 1
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNCTMNFKNNNGNNIGLLGFKADTVVASTWYYTHMRDNTNSNGFFWNFISEEHGWQEK

SEQ ID NO: 25 - protein, 1251 aa, Clostridium butyricum 2
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFVPELLIPTILVFTIKSFLGSSDNKNKVIKAINNALKERDEKWKEVYSFIVSNWMTKINTQFNKRKEQMYQALQNQVNALKTIIEFKYNSYTLEEKKELKNNYDIEQIENELNQKVSIAMNNIDRFLTESSISYLMKLINEVKINKLREYDENVKTYLLDYIIQHGSILGESQQELNSMVIDTLNNSIPFKLSSYTDDKILISYFNKFFKRIKSSSVLNMRYKNDKYVDTSGYDSNININGEIFIYPTNKNQFTIFNSKPSEVNISQNDYIIYDNKYKNFSISFWVRIPNYDNKIVNINNEYTIINCMRDNNSGWKVSLNHNEIIWTLQDNARINQKLVFKYGNANGISDYINKWIFVTITNDRLGDSKLYINGHLIDQKSILNLGNIHVSDNILFKIVNCSYTRYIGIRYFNIFDKELDETEIQTLYSNEPNTNILKDFWGNYLLYDKGYYLLNVLKPNNFIDRRKDSTLSINNIRSTILLANRLYSGIKVKIQRVNDSSTNDRFVRKNDQVYINYISNSSSYSLYADTNTTDKEKTIKSSSSGNRFNQVVVMNSVGNNCTMNFKNNNGNNIGLLGFKADTVVASTWYYTHMRDHTNSNGCFWNFISEEHGWQEK

SEQ ID NO: 26 - protein, 25 aa, Artificial Sequence
  BoNT/A di-chain loop region
CVRGIITSKTKSLDKGYNKALNDLC

SEQ ID NO: 27 - protein, 10 aa, Artificial Sequence
  BoNT/B di-chain loop region
CKSVKAPGIC

SEQ ID NO: 28 - protein, 17 aa, Artificial Sequence
  BoNT/C1 di-chain loop region
CHKAIDGRSLYNKTLDC

SEQ ID NO: 29 - protein, 14 aa, Artificial Sequence
  BoNT/D di-chain loop region
CLRLTKNSRDDSTC

SEQ ID NO: 30 - protein, 15 aa, Artificial Sequence
  BoNT/E di-chain loop region
CKNIVSVKGIRKSIC

SEQ ID NO: 31 - protein, 17 aa, Artificial Sequence
  BoNT/F di-chain loop region
CKSVIPRKGTKAPPRLC

SEQ ID NO: 32 - protein, 15 aa, Artificial Sequence
  BoNT/G di-chain loop region
CKPVMYKNTGKSEQC

SEQ ID NO: 33 - protein, 29 aa, Artificial Sequence
  TeNT di-chain loop region
CKKIIPPTNIRENLYNRTASLTDLGGELC

SEQ ID NO: 34 - protein, 15 aa, Artificial Sequence
  BaNT di-chain loop region
CKSIVSKKGTKNSLC

SEQ ID NO: 35 - protein, 15 aa, Artificial Sequence
  BuNT di-chain loop region
CKNIVSVKGIRKSIC

SEQ ID NO: 36 - protein, 5 aa, Artificial Sequence
  Bovine enterokinase protease cleavage site
DDDDK

SEQ ID NO: 37 - protein, 7 aa, Artificial Sequence
  Tobacco Etch Virus protease cleavage site consensus sequence
EXXYXQG

SEQ ID NO: 38 - protein, 7 aa, Artificial Sequence
  Tobacco Etch Virus protease cleavage site consensus sequence
EXXYXQS

SEQ ID NO: 39 - protein, 7 aa, Artificial Sequence
  Tobacco Etch Virus protease cleavage site
ENLYFQG

SEQ ID NO: 40 - protein, 7 aa, Artificial Sequence
  Tobacco Etch Virus protease cleavage site
ENLYFQS

SEQ ID NO: 41 - protein, 7 aa, Artificial Sequence
  Tobacco Etch Virus protease cleavage site
ENIYTQG

SEQ ID NO: 42 - protein, 7 aa, Artificial Sequence
  Tobacco Etch Virus protease cleavage site
ENIYTQS

SEQ ID NO: 43 - protein, 7 aa, Artificial Sequence
  Tobacco Etch Virus protease cleavage site
ENIYLQG

SEQ ID NO: 44 - protein, 7 aa, Artificial Sequence
  Tobacco Etch Virus protease cleavage site
ENIYLQS

SEQ ID NO: 45 - protein, 7 aa, Artificial Sequence
  Tobacco Etch Virus protease cleavage site
ENVYFQG

SEQ ID NO: 46 - protein, 7 aa, Artificial Sequence
  Tobacco Etch Virus protease cleavage site
ENVYSQS

SEQ ID NO: 47 - protein, 7 aa, Artificial Sequence
  Tobacco Etch Virus protease cleavage site
ENVYSQG

SEQ ID NO: 48 - protein, 7 aa, Artificial Sequence
  Tobacco Etch Virus protease cleavage site
ENVYSQS

SEQ ID NO: 49 - protein, 7 aa, Artificial Sequence
  Tobacco Vein Mottling Virus protease cleavage site consensus sequence
XXVRFQG

SEQ ID NO: 50 - protein, 7 aa, Artificial Sequence
  Tobacco Vein Mottling Virus protease cleavage site consensus sequence
XXVRFQS

SEQ ID NO: 51 - protein, 7 aa, Artificial Sequence
  Tobacco Vein Mottling Virus protease cleavage site
ETVRFQG

SEQ ID NO: 52 - protein, 7 aa, Artificial Sequence
  Tobacco Vein Mottling Virus protease cleavage site
ETVRFQS

SEQ ID NO: 53 - protein, 7 aa, Artificial Sequence
  Tobacco Vein Mottling Virus protease cleavage site
NNVRFQG

SEQ ID NO: 54 - protein, 7 aa, Artificial Sequence
  Tobacco Vein Mottling Virus protease cleavage site
NNVRFQS

SEQ ID NO: 55 - protein, 7 aa, Artificial Sequence
  Human Rhinovirus 3C protease cleavage site consensus sequence
XXLFQGP

SEQ ID NO: 56 - protein, 7 aa, Artificial Sequence
  Human Rhinovirus 3C protease cleavage site
EALFQGP

SEQ ID NO: 57 - protein, 7 aa, Artificial Sequence
  Human Rhinovirus 3C protease cleavage site
EVLFQGP

SEQ ID NO: 58 - protein, 7 aa, Artificial Sequence
  Human Rhinovirus 3C protease cleavage site
ELLFQGP

SEQ ID NO: 59 - protein, 7 aa, Artificial Sequence
  Human Rhinovirus 3C protease cleavage site
DALFQGP

SEQ ID NO: 60 - protein, 7 aa, Artificial Sequence
  Human Rhinovirus 3C protease cleavage site
DVLFQGP

SEQ ID NO: 61 - protein, 7 aa, Artificial Sequence
  Human Rhinovirus 3C protease cleavage site
DLLFQGP

SEQ ID NO: 62 - protein, 6 aa, Artificial Sequence
  Subtilisin cleavage site consensus sequence
XXXXHY

SEQ ID NO: 63 - protein, 6 aa, Artificial Sequence
  Subtilisin cleavage site consensus sequence
XXXXYH

SEQ ID NO: 64 - protein, 2 aa, Artificial Sequence
  Subtilisin cleavage site
HY

SEQ ID NO: 65 - protein, 2 aa, Artificial Sequence
  Subtilisin cleavage site
YH

SEQ ID NO: 66 - protein, 6 aa, Artificial Sequence
  Subtilisin cleavage site
PGAAHY

SEQ ID NO: 67 - protein, 6 aa, Artificial Sequence
  Hydroxylamine cleavage site
NGNGNG

SEQ ID NO: 68 - protein, 2 aa, Artificial Sequence
  Hydroxylamine cleavage site
NG

SEQ ID NO: 69 - protein, 5 aa, Artificial Sequence
  SUMO/ULP-1 protease cleavage site consensus sequence
GGXXX

SEQ ID NO: 70 - protein, 98 aa, Artificial Sequence
  SUMO/ULP-1 protease cleavage site
MADSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 71 - protein, 5 aa, Artificial Sequence
  Caspase 3 protease cleavage site consensus sequence
DXXDX

SEQ ID NO: 72 - protein, 5 aa, Artificial Sequence
  Caspase 3 protease cleavage site
DEVDG

SEQ ID NO: 73 - protein, 5 aa, Artificial Sequence
  Caspase 3 protease cleavage site
DEVDS

SEQ ID NO: 74 - protein, 5 aa, Artificial Sequence
  Caspase 3 protease cleavage site
DEPDG

SEQ ID NO: 75 - protein, 5 aa, Artificial Sequence
  Caspase 3 protease cleavage site
DEPDS

SEQ ID NO: 76 - protein, 5 aa, Artificial Sequence
  Caspase 3 protease cleavage site
DELDG

SEQ ID NO: 77 - protein, 5 aa, Artificial Sequence
  Caspase 3 protease cleavage site
DELDS

SEQ ID NO: 78 - protein, 4 aa, Artificial Sequence
  Flexible G-spacer
GGGG

SEQ ID NO: 79 - protein, 5 aa, Artificial Sequence
  Flexible G-spacer
GGGGS

SEQ ID NO: 80 - protein, 4 aa, Artificial Sequence
  Flexible A-spacer
AAAA

SEQ ID NO: 81 - protein, 5 aa, Artificial Sequence
  Flexible A-spacer
AAAAV

SEQ ID NO: 82 - protein, 12 aa, Artificial Sequence
  PSMA Binding Peptide
WQPDTAHHWATL

SEQ ID NO: 83 - protein, 18 aa, Artificial Sequence
  PSMA Binding Peptide
MAEWQPDTAHHWATLPDP

SEQ ID NO: 84 - protein, 12 aa, Artificial Sequence
  PSMA Binding Peptide
HNAYWHWPPSMT

SEQ ID NO: 85 - protein, 12 aa, Artificial Sequence
  PSMA Binding Peptide
GHLIPLRQPSHQ

SEQ ID NO: 86 - protein, 12 aa, Artificial Sequence
  PSMA Binding Peptide
YTSPHHSTTGHL

SEQ ID NO: 87 - protein, 12 aa, Artificial Sequence
  PSMA Binding Peptide
WTHHHSYPRPLQ

SEQ ID NO: 88 - protein, 12 aa, Artificial Sequence
  PSMA Binding Peptide
NSFPLMLMHHHP

SEQ ID NO: 89 - protein, 12 aa, Artificial Sequence
  PSMA Binding Peptide
KHMHWHPPALNT

SEQ ID NO: 90 - protein, 12 aa, Artificial Sequence
  PSMA Binding Peptide
SLDSMSPQWHAD

SEQ ID NO: 91 - protein, 12 aa, Artificial Sequence
  PSMA Binding Peptide
SEFIHHWTPPPS

SEQ ID NO: 92 - protein, 12 aa, Artificial Sequence
  PSMA Binding Peptide
NGFSHHAPLMRY

SEQ ID NO: 93 - protein, 12 aa, Artificial Sequence
  PSMA Binding Peptide
HHPWTHHWPPPP

SEQ ID NO: 94 - protein, 12 aa, Artificial Sequence
  PSMA Binding Peptide
HHFWTHHWPPPP

SEQ ID NO: 95 - protein, 12 aa, Artificial Sequence
  PSMA Binding Peptide
AWPENPSRRPFT

SEQ ID NO: 96 - protein, 12 aa, Artificial Sequence
  PSMA Binding Peptide
AGFQHHPSFYRF

SEQ ID NO: 97 - protein, 12 aa, Artificial Sequence
  PSMA Binding Peptide
KSLSRHDHIHHH

SEQ ID NO: 98 - protein, 12 aa, Artificial Sequence
  PSMA Binding Peptide
YRHWPIDYPPPP

SEQ ID NO: 99 - protein, 12 aa, Artificial Sequence
  PSMA Binding Peptide
MIHTNHWWAQDR

SEQ ID NO: 100 - protein, 12 aa, Artificial Sequence
  PSMA Binding Peptide
QRSPMMSRIRLP

SEQ ID NO: 101 - protein, 12 aa, Artificial Sequence
  PSMA Binding Peptide
TPLPSFTDGHHT

SEQ ID NO: 102 - protein, 9 aa, Artificial Sequence
  PSMA Binding Peptide
CTITSKRTC

SEQ ID NO: 103 - protein, 9 aa, Artificial Sequence
  PSMA Binding Peptide
CTLVPHTRC

SEQ ID NO: 104 - protein, 7 aa, Artificial Sequence
  PSMA Binding Peptide
QKHHNYL

SEQ ID NO: 105 - protein, 7 aa, Artificial Sequence
  PSMA Binding Peptide
TITSKRT

SEQ ID NO: 106 - protein, 7 aa, Artificial Sequence
  PSMA Binding Peptide
TITAKRT

SEQ ID NO: 107 - protein, 8 aa, Artificial Sequence
  PSMA Binding Peptide
PTITSKRT

SEQ ID NO: 108 - protein, 8 aa, Artificial Sequence
  PSMA Binding Peptide
PTITAKRT

SEQ ID NO: 109 - protein, 7 aa, Artificial Sequence
  PSMA Binding Peptide
TLVPHTR

SEQ ID NO: 110 - protein, 7 aa, Artificial Sequence
  PSMA Binding Peptide
TLVPHSR

SEQ ID NO: 111 - protein, 7 aa, Artificial Sequence
  PSMA Binding Peptide
NLVPHTR

SEQ ID NO: 112 - protein, 7 aa, Artificial Sequence
  PSMA Binding Peptide
NLVPHSR

SEQ ID NO: 113 - protein, 268 aa, Artificial Sequence
  Recombinant Green Fluorescent Protein (GFP)
MEGPVTGTGSRYLGGRSASFANSGGGGGASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNID

SEQ ID NO: 114 - DNA, 804 bp, Artificial Sequence
  Open reading frame encoding recombinant Green Fluorescent Protein (GFP)
atggagggcccggttaccggtaccggatccagatatctgggcggccgctcagcaagcttcgcgaattcgggaggcggaggtggagctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaacatcgat

SEQ ID NO: 115 - protein, 710 aa, Artificial Sequence
  Recombinant Green Fluorescent Protein (GFP)- BoNT/A light chain fusion protein
MEGPVTGTGSRYLGGRSASFANSGGGGGASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNIDGGGGGPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 116 - DNA, 2130 bp, Artificial Sequence
  Open Reading Frame of recombinant Green Fluorescent Protein-BoNT/A light chain
atggagggcccggttaccggtaccggatccagatatctgggcggccgctcagcaagcttcgcgaattcgggaggcggaggtggagctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaacatcgatggaggcggaggtggaccttttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataatcacttcgaaa

SEQ ID NO: 117 - protein, 694 aa, Artificial Sequence
  Recombinant Green Fluorescent Protein (GFP)- BoNT/B light chain fusion protein
MASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNIDGGGGGKGPVTGTGSPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSV

SEQ ID NO: 118 - DNA, 2082 bp, Artificial Sequence
  Open Reading Frame of recombinant Green Fluorescent Protein-BoNT/B light chain
atggctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaacatcgatggaggcggaggtggaaagggcccggttaccggtaccggatccccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgtt

SEQ ID NO: 119 - protein, 706 aa, Artificial Sequence
  Recombinant Green Fluorescent Protein (GFP)- BoNT/C1 light chain fusion protein
MASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNIDGGGGGKGPVTGTGDVSIMPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSNSD

SEQ ID NO: 120 - DNA, 2118 bp, Artificial Sequence
  Open Reading Frame of recombinant Green Fluorescent Protein-BoNT/C1 light chain
atggctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaacatcgatggaggcggaggtggaaagggcccggttaccggtaccggagatgttagtattatgccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcgaattctgac

SEQ ID NO: 121 - protein, 681 aa, Artificial Sequence
  Recombinant Green Fluorescent Protein (GFP)- BoNT/E light chain fusion protein
MASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNIDGGGGGKGPVTGTGSPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKLREF

SEQ ID NO: 122 - DNA, 2043 bp, Artificial Sequence
  Open Reading Frame of recombinant Green Fluorescent Protein-BoNT/E light chain
atggctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaacatcgatggaggcggaggtggaaagggcccggttaccggtaccggatccccaaaaattaatagttttaattataatgatcctgttaatgatagaacaattttatatattaaaccaggcggttgtcaagaattttataaatcatttaatattatgaaaaatatttggataattccagagagaaatgtaattggtacaaccccccaagattttcatccgcctacttcattaaaaaatggagatagtagttattatgaccctaattatttacaaagtgatgaagaaaaggatagatttttaaaaatagtcacaaaaatatttaatagaataaataataatctttcaggagggattttattagaagaactgtcaaaagctaatccatatttagggaatgataatactccagataatcaattccatattggtgatgcatcagcagttgagattaaattctcaaatggtagccaagacatactattacctaatgttattataatgggagcagagcctgatttatttgaaactaacagttccaatatttctctaagaaataattatatgccaagcaatcacggttttggatcaatagctatagtaacattctcacctgaatattcttttagatttaatgataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggaagcttcgcgaattc